(19) United States
(12) Patent Application Publication
(10) Pub. No.: US 2021/0113715 A1
(43) Pub. Date: Apr. 22, 2021

(54) TARGETED NANOPARTICLES FOR DIAGNOSING, DETECTING AND TREATING CANCER

(71) Applicant: CEDARS-SINAI MEDICAL CENTER, Los Angeles, CA (US)

(72) Inventors: J. Manuel Perez, West Hollywood, CA (US); Leland Chung, Beverly Hills, CA (US); Yi Zhang, Los Angeles, CA (US); Keith L. Black, Los Angeles, CA (US)

(73) Assignee: CEDARS-SINAI MEDICAL CENTER, Los Angeles, CA (US)

(21) Appl. No.: 17/047,638

(22) PCT Filed: Apr. 18, 2019
(86) PCT No.: PCT/US2019/028196

§ 371 Date: Oct. 14, 2020

Related U.S. Application Data
(60) Provisional application No. 62/731,671, filed on Sep. 14, 2018.

(51) Int. Cl. A61K 49/00; A61K 49/12; A61K 49/18; A61K 31/337; A61K 31/69; A61K 31/47; A61K 31/365; A61K 45/06; A61P 35/00

(52) U.S. Cl. CPC A61K 49/0002; A61K 49/0032; A61K 49/0093; A61K 49/126; A61K 49/1818; A61P 35/00; A61K 31/69; A61K 31/47; A61K 31/365; A61K 45/06; A61K 31/337

(57) ABSTRACT

The present invention provides a nanoparticle, comprising: a core, wherein the core comprises at least one iron oxide; a shell surrounding the core, wherein the shell comprises at least one polymer; and at least one targeting moiety attached to the shell, wherein the nanoparticle does not comprise boron, for use in methods for detecting and treating cancer in a subject.

### CROSS-REFERENCE TO RELATED APPLICATIONS

This application claims priority under 35 U.S.C. § 119(e) to U.S. provisional patent application No. 62/731,671 filed Sep. 14, 2018, the entirety of which is hereby incorporated by reference.

### STATEMENT REGARDING FEDERALLY SPONSORED RESEARCH OR DEVELOPMENT

This invention was made with government support under Grant No. EB019288 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Apr. 17, 2019, is named SequenceListing-065472-000662WO00_ST25.txt and is 1,736 bytes in size.

### FIELD OF THE INVENTION

Embodiments of the invention are related to nanoparticles and to the use thereof for the diagnosis, detection, and treatment of cancer.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Many people suffer from cancer and require treatment. As such there is a need for improved cancer diagnosis and detection, and for improved therapies for the treatment of cancer. The present invention addresses that need.

### SUMMARY OF THE INVENTION

The following embodiments and aspects thereof are described and illustrated in conjunction with systems, compositions, articles of manufacture, and methods which are meant to be exemplary and illustrative, not limiting in scope.

In various embodiments, the present invention provides a nanoparticle, comprising: a core, wherein the core comprises at least one iron oxide; a shell surrounding the core, wherein the shell comprises at least one polymer; and at least one targeting moiety attached to the shell, wherein the nanoparticle does not comprise boron.

In various embodiments, the present invention provides method for detecting and treating a cancer in a subject, comprising: administering an effective amount of at least one nanoparticle of the present invention to the subject, wherein the at least one nanoparticle comprises at least one drug, thereby contacting a tissue of the subject with the at least one nanoparticle such that the at least one nanoparticle binds to the tissue; detecting the at least one nanoparticle bound to the tissue, wherein the presence of the at least one nanoparticle bound to the tissue is indicative of the cancer in the subject; and delivering the at least one drug to the tissue thereby treating the cancer in the subject.

In various embodiments, the present invention provides a method for detecting a cancer in a subject, comprising: administering an effective amount of at least one nanoparticle of the present invention to the subject, thereby contacting a tissue of the subject with the at least one nanoparticle such that the at least one nanoparticle binds to the tissue; and detecting the at least one nanoparticle bound to the tissue, wherein the presence of the at least one nanoparticle bound to the tissue is indicative of the cancer in the subject.

In various embodiments, the present invention provides a probe comprising at least one coated iron oxide nanoparticle; and at least one targeting moiety, wherein the probe does not comprise boron.

### BRIEF DESCRIPTION OF THE DRAWINGS

Exemplary embodiments are illustrated in referenced figures. It is intended that the embodiments and figures disclosed herein are to be considered illustrative rather than restrictive.

FIG. 1 depicts in accordance with various embodiments of the invention, an HMC-FH platform technology can be used to facilitate the pre-operative MRI and intraoperative fluorescent assessment of tumor margins. The same nanoparticle technology can be used to deliver drugs to tumors via HMC-FH(Drug), where FH is Feraheme and Drug is encapsulated within the carboxymethyl dextran coating on FH.

FIG. 2 depicts in accordance with various embodiments of the invention, Heptamethine cyanine (HMC) dyes and conjugates. The near infrared dye and OATP-targeting ligand HMC can be conjugated with a lysine linker to yield HMC-Lys, which can then be conjugated to carboxylic acid groups on Feraheme (FH). The HMC dye binds to the OATP receptor in cancer cells. HMC has near infrared fluorescence (ex/em 750/800). Therefore, an HMC-FH nanoprobe will target cancer cells via the OATP receptor, labeling the tumor with iron oxide for MR Imaging and fluorescent for intraoperative surgery. When a particular drug is encapsulated in the HMC-FH nanocarrier, the resulting HMC-FH(Drug) will deliver the drug to tumor, causing tumor regression and improved survival.

FIG. 3A-FIG. 3D depicts in accordance with various embodiments of the invention, NIRF and MRI characterization of HMC-FH. Bright field and SIRIS NIRF images of FH and HMC-FH showing their aqueous stability and bright fluorescent for the HMC-FH (FIG. 3A). Dose dependent and 1-week stability comparison studies of the nanoparticle formulations (FIG. 3B). Serial dilution of HMC-FH showing that the SIRIS system can detect down to 400 nm of HMC-FH within a cell pellet (FIG. 3C, top row); also, this amount of HMC-FH (400 nm) can detect down to 5K cells in vitro using SIRIS (FIG. 3C, bottom row). Magnetic relaxation of the FH formulation (FIG. 3D, insert) and cell quantification detection limit by MRI (FIG. 3D, graph).

FIG. 4A-FIG. 4B depicts in accordance with various embodiments of the invention, Targeting of HMC-FH to PCa cells and tumors. HMC-FH internalizes in PCa cells, fluorescently labeling the cytoplasm (FIG. 4A). In vivo studies using PCa mouse subcutaneous xenographs showing specific targeting of tumors in vivo (FIG. 4B).

FIG. 5A-FIG. 5F depicts in accordance with various embodiments of the invention, MRI and NIRF(SIRIS) visualization of an 22Rv1 orthotopic prostate model. Two adjacent tumors are clearly visualized on the right lobe of the mouse prostate (FIG. 5A). NIRF images using the IVIS (FIG. 5B) and SIRIS (FIG. 5C) clearly indicate localization of fluorescent HMC-FH to the prostate's right lobe. Intraoperative visualization using SIRIS clearly show a brightly fluorescent tumor with clearly visible tumor margins (FIG. 5D) and the presence of two adjacent tumors (FIG. 5E). Histopathology confirms the specific localization of fluorescent nanoparticles to the tumor area (FIG. 5F).

FIG. 6 depicts in accordance with various embodiments of the invention, 22Rv1 tumor growth inhibition of cabozantanib (cabo) and HMC-FH(cabo) treated mice. Injected dose HMC-FH(DXL): 4 ug Fe/g of mice (4 mg Fe/Kg). 0.5 ug DXL/g of mice (0.5 mg DXL/Kg). Injected dose DXL 0.5 ug DXL/g of mice (0.5 mg DXL/Kg). HMC-FH (DXL) treated mice had a significantly slower (p≤0.0001) tumor growth curve, compared with non treated control mice (PBS) or mice treated with DXL along.

FIG. 7 depicts in accordance with various embodiments of the invention, 22Rv1 tumor growth inhibition of docetaxel (DXL) and HMC-FH(DXL) treated mice. Injected dose HMC-FH(cabo): 4 ug Fe/g of mice (4 mg Fe/Kg). 0.5 ug cabo/g of mice (0.5 mg cabo/Kg). Injected dose cabo: 0.5 ug cabo/g of mice (0.5 mg cabo/Kg). HMC-FH (cabo) treated mice had a significantly slower (p≤0.0001) tumor growth curve, compared with non treated control mice (PBS) or mice treated with cabo along.

FIG. 8A-FIG. 8C depicts in accordance with various embodiments of the invention, PC3 prostate cancer cells exhibit decreased migration in the presence of BFA and HMC-FH (BFA): PC3 cells (5×10⁴) in serum-free RPMI medium were added to upper chambers of transwell inserts and allowed to migrate to the bottom chamber of the apparatus contained media with 10% FBS, for 24 h at 37° C. After incubation, nonmigratory cells and media were washed from transwells, and those cells that migrated to the bottom of the filters were, fixed and stained and imaged using a fluorescence Microscope. Representative images (5 fields) of Control vs HMC-FH(BFA) (10 uM) (FIG. 8A) and HMC-FH vs BFA (FIG. 8B). Note the crease level of cell migration of cells treated with HMC-FH(BFA) or BFA along. Quantification of the average number of cells per image that have migrated (FIG. 8C). HMC-FH (BFA) treated wells had a significant (p≤0.0001) decrease in migration, compared with cells treated with either BFA alone, HMC-FH or DMSO control.

FIG. 9A-FIG. 9B depicts in accordance with various embodiments of the invention, LNCaP (FIG. 9A) and PC3 (FIG. 9B) prostate cancer cells exhibit decreased migration in the presence of DXT and HMC-FH (DXT): PC3 or LNCaP cells (5×10⁴) in serum-free RPMI medium were added to upper chambers of transwell inserts and allowed to migrate to the bottom chamber of the apparatus contained media with 10% FBS, for 24 h at 37° C. HMC-FH (DXT) treated wells had a significant (p≤0.0001) decrease in migration, compared with cells treated with either DXT alone, HMC-FH or DMSO control.

FIG. 10 depicts in accordance with various embodiments of the invention, Brightfield and Near Infrared fluorescence microcopy images GBM cell lines treated with HMC-FH for 24 hours Within 24 H, near infrared fluorescence is observed throughout the each one of the cells studied.

FIG. 11A-FIG. 11B depicts in accordance with various embodiments of the invention, Near Infrared Images of Mice with Intracraneal U87 Tumors after injection with HMC-FH for 24 H (FIG. 11A) or 7 days (FIG. 11B) with corresponding images of organs after necroscopy. Within 24 H, near infrared fluorescence is observed throughout the mouse and in every organ. Within the brain, most of the fluorescence resides within the tumor. In 7 days, most of the fluorescence remains within the brain tumor, with no to minimal fluorescence in the other organs.

FIG. 12A-FIG. 12F depicts in accordance with various embodiments of the invention, Near infrared visualization of a mouse brain with a U87 intracraneal tumor. Representative image of a mouse brain from a mouse that had previously been injected with HMC-FH and imaged with a house built near infrared camera, 24 h after injection. White light (FIG. 12A) and corresponding merging with fluorescent light (FIG. 12B) images of a mouse brain with a U87 intracraneal tumor. Series of snapshots showing removal of the brain tumor from the mouse brain (FIG. 12C-FIG. 12F), clearly showing the presence of a brightly fluorescent brain tumor with clearly visible tumor margins.

FIG. 13A-FIG. 13C depicts in accordance with various embodiments of the invention, Post near infrared visualization of a mouse brain with a U87 intracraneal tumor after tumor removal. Representative image of a mouse brain that had previously been injected with HMC-FH and imaged with a house built near infrared camera, 24 h after injection. White light image of the brain and the extracted tumor (FIG. 13A). Notice that not much difference is observed between the two, except for the fact that the brain mass appears darker. Corresponding near infrared image (FIG. 13B) showing a brightly fluorescent tumor and what looks like perhaps residual infiltrating tumors left in the brain mass. Corresponding white light and fluorescent merge image (FIG. 13C).

FIG. 14A-FIG. 14C depicts in accordance with various embodiments of the invention, Histology of a mouse brain with a U87 intracraneal tumor. Brightfield (FIG. 14A), H&E (FIG. 14B) and near infrared (FIG. 14C) images of a mouse brain with a U87 intracraneal tumor. Notice a strong co-localization of near infrared fluorescence and the areas stained by H&E.

FIG. 15A-FIG. 15D depicts in accordance with various embodiments of the invention, Histology of a U87 intracraneal tumor border. Brightfield (FIG. 15A), DAPI (FIG. 15B), Near Infrared Fluorescence (FIG. 15C), and merged (FIG. 15D) images of the tumor border. Notice a strong localization of near infrared fluorescence (nanoparticles) in the tumor area, with minimal localization outside the tumor borders.

FIG. 16 depicts in accordance with various embodiments of the invention, Histology of a U87 intracraneal tumor border indicating crossing of the brain blood barrier (BBB). Brain tissue slides were stained for DAPI (blue, nuclear stain) and von Willebrand factor (cWF, green, vascular endothelium). None of the NRF signal (red, for the HMC-FH nanoparticles) is associated with the vWF signal (green, for the vascular endothelial cells), indicating crossing of the BBB in the tumor area. In addition, the red signal outside the tumor area is not associated with green signal, indicating that near the tumor borders the nanoparticles are not trapped within the endothelium (vasculature) and they have crossed the BBB.

FIG. 17A-FIG. 17B depicts in accordance with various embodiments of the invention, Survival Studies (Kaplan-Meire Curve) of Mice (n=5) with Intracraneal U87 tumors treated 14 days after tumor implantation. A dose of 3 umol drug/kg, 22 mM Fe (FH) was administered i.v via tail vein injection twice a week for two weeks. A longer survival was observed in mice treated with the HMC-FH encapsulated drugs in contrast with the drug along. Mice treated with HMC-FH(PXT) (FIG. 17A) had a statistically significant longer survival than mice treated with HMC-FH(DXT) (FIG. 17B).

FIG. 18 depicts in accordance with various embodiments of the invention, Survival Studies (Kaplan-Meire Curve) of Mice (n=10) with intracraneal U87 tumors treated 5 days after tumor implantation with HMC-FH(PXL). A dose of 3 umol drug/kg, 22 mM Fe (FH) was administered i.v. via tail vein injection twice a week for three weeks. The survival of mice treated HMC-FH(PXL) was significantly longer than those observed with the FH(PXL), PXL alone, or the PBS (control) mice.

FIG. 19 depicts in accordance with various embodiments of the invention, Survival Studies (Kaplan-Meire Curve) of Mice (n=5) with intracraneal U87 tumors treated 14 days after tumor implantation with HMC-FH(BFA). A dose of 3 umol drug/kg, 22 mM Fe (FH) was administered i.v. via tail vein injection twice a week for three weeks. The survival of mice treated HMC-FH(BFA) and BFA along was significantly longer than in the control mice.

FIG. 20 depicts in accordance with various embodiments of the invention, U87R cells exhibit decreased migration in the presence of BFA and HMC-FH (BFA): TMZ-resistant U87R cells (2×10⁴) in serum-free DMEM medium were added to upper chambers of transwell inserts and allowed to migrate to the bottom chamber of the apparatus contained media with 10% FBS, for 24 h at 37° C. After incubation, nonmigratory cells and media were washed from transwells, and those cells that migrated to the bottom of the filters were, fixed and stained and imaged using a fluorescence Microscope (Keyence BZ-X7 00). Representative images (5 fields) were taken of treatment (2 uM of each-BFA, HMC-FH and HMC-FH (BFA, DMSO) for quantification.

FIG. 21 depicts in accordance with various embodiments of the invention, Low molecular weight PSMA-targeting glutamate urea based probe. F—N—[N—[(S)-1,3-dicarboxypropyl]carbamoyl]-4-fluorobenzyl-1-cysteine (18F-DCFBC).

FIG. 22 depicts in accordance with various embodiments of the invention, PSMA-targeting Feraheme nanoparticle. The iron oxide core (TO) is surrounded by a polymeric coating such as carboxymethyl dextran, where carboxylic groups are conjugated to either Glutamate (Glu) or Folate (Fol) to yield two Feraheme-based MRI probe to image PSMA by MRI.

FIG. 23 depicts in accordance with various embodiments of the invention, Theranostics HM-Feraheme (BF) nanoparticle. A lipophilic drug, such as Brefeldin, is encapsulated within the carboxymethyl dextran coating of either Glu-Feraheme or Fol-Feraheme. The resulting nanoparticle with dual therapeutic and imaging can deliver drugs to cancer cells via PSMA, while being able to visualize drug-nanoparticle localization in tissue by imaging methods.

FIG. 24 depicts in accordance with various embodiments of the invention, Microscopy images of prostate cancer cell lines treated with Glu-Feraheme (BF). Cell death is seen in CWR22v1 and LNCaP, which are PSMA positive cell lines, while no significant cell death is seen in the DU145 and PC3 cells which are PSMA negative. Dose: 2 ug BFA/mL.

FIG. 25 depicts in accordance with various embodiments of the invention, Cell detachment of PSMA positive prostate cancer cells treated with Glu-Feraheme (BF). Time response cell detachment is seen in the PSMA positive LNCaP cells but not in PC3, which are PSMA negative. Dose: 2 ug BFA/mL.

FIG. 26 depicts in accordance with various embodiments of the invention, Microscopy images of normal prostate epithelial cells treated with Glu-Feraheme (BF). No significant change in cell morphology or cytotoxicity is observed in the treated cells versus the non-treated control. Dose: 2 ug BFA/mL.

FIG. 27 depicts in accordance with various embodiments of the invention, Angiopep-Feraheme nanoparticles. The iron oxide core (IO) is surrounded by a polymeric coating such as carboxymethyl dextran that can encapsulate a drug or near infrared dye as cargo, and where carboxylic acid groups are conjugated to Angiopep to facilitate crossing of the BBB and uptake by glioblastoma cells. This yields two formulation used in our studies: Angiopep-Feraheme (BFA) and Angiopep-Feraheme (DiI).

FIG. 28 depicts in accordance with various embodiments of the invention, Conjugation of Angiopep-Cysteine (TFFYGGSRGKRNNFKTEEYC) (SEQ ID NO: 1) onto Feraheme carboxylic acid groups. A Maleimide-PEG-Amine linker was first conjugated to the carboxylic acid group on Feraheme to yield a Maleimide-PEG-Feraheme before reaction with the Angiopep-Cysteine peptide.

FIG. 29 depicts in accordance with various embodiments of the invention, Internalization and effect of Angiopep-Feraheme (DiI) and Angiopep-Feraheme (BFA) on HBMVEC cells. A significant amount of cell associated fluorescence was observed in Angiopep-Feraheme (DiI) treated HBMVEC, whereas cells treated with Feraheme (DiI) did not results in any fluorescence. This indicates that Angiopep facilitated the internalization of these nanoparticles into the cells. Meanwhile, when BFA as a model drug was encapsulated into the nanoparticles, no significant toxicity was observed either, as approximately 80% of viable cells remained after treatment. 24 h treatment, 550 nm BFA.

FIG. 30 depicts in accordance with various embodiments of the invention, Internalization and effect of Angiopep-Feraheme (DiI) and Angiopep-Feraheme (BFA) on U87 cells. A significant amount of cell associated fluorescence was observed in Angiopep-Feraheme (DiI) treated U87 GBM cells, with no observable toxicity. However, when BFA encapsulated nanoparticles (Angiopep-Feraheme (DiI)) were used, significant changes in cell morphology and cell death was observed. 48 h treatment, 550 nm BFA.

FIG. 31 depicts in accordance with various embodiments of the invention, Flow cytometry studies of BFA-Feraheme nanoparticles. After 48 hours of treatment of U87 cells with Feraheme (BFA), 81 percent of the cells remained viable. However, when the corresponding nanoparticles with Angiopep were used, this number was reduced to 24% of viable cells. 48 h treatment, 550 nm BFA.

FIG. 32 depicts in accordance with various embodiments of the invention, Microscopy images of control, and Angiopep-Feraheme (BFA) treated CSC55 GBM Stem Cells. Internalization of the Angiopep-Feraheme (DiD) was corroborated by observation of cell associated fluorescence (DiI) in the treated cells. Furthermore, Angiopep-Feraheme (BFA) inhibits stem cell colonization and the stability of these colonies when they are formed.

FIG. 33 depicts in accordance with various embodiments of the invention, Flow cytometry studies of BFA-Feraheme nanoparticles. After 5 days of treatment of CSC55 stem cells, Feraheme (BFA), 82% of the cells remained viable. However, when the corresponding nanoparticles with Angiopep were used, this number was reduced to 6.96% of viable cells. 5 days treatment, 550 nm BFA.

FIG. 34 depicts in accordance with various embodiments of the invention, Multimodal HM-Feraheme nanoparticle. The iron oxide core (IO) is surrounded by a polymeric coating such as carboxymethyl dextran, where carboxylic groups are conjugated to a heptamethine (HM), generating a nanoparticle with dual fluorescent and magnetic properties that target the OATP receptor in cancer cells.

FIG. 35 depicts in accordance with various embodiments of the invention, Theranostics HM-Feraheme (BF) nanoparticle. A lipophilic drug, such as Brefeldin, is encapsulated within the carboxymethyl dextran coating of HM-Feraheme. The resulting nanoparticle with dual therapeutic and imaging (fluorescent and MRI) can deliver drugs to cancer cells via the OATP receptor, while being able to visualize drug-nanoparticle localization in tissue by imaging methods.

FIG. 36 depicts in accordance with various embodiments of the invention, Conjugation of Heptamethine to Feraheme carboxylic acid groups. A heptamethine-lysine conjugate (HM-Lys-NH₂) was conjugated to the available carboxylic acid groups on the surface of Feraheme using EDC/NHS chemistry.

FIG. 37 depicts in accordance with various embodiments of the invention, Fluorescence Imaging (EX/EM) of prostate cancer cell lines incubated with HM-Feraheme for 12 hours.

FIG. 38 depicts in accordance with various embodiments of the invention, In vivo fluorescence imaging of mice after 24, 28 and 120 h post injection of the HM-Feraheme dye. Yellow arrows indicate localization of the tumors.

FIG. 39 depicts in accordance with various embodiments of the invention, Near Infrared Fluorescence Organ Biodistribution on Excised tissues. Notice the higher tumor associated fluorescence compared with the rest of the tissues, suggesting a larger tumor accumulation of the nanoparticles.

FIG. 40A-FIG. 40B depicts in accordance with various embodiments of the invention, NIRF characterization of HMC-FH. Brightfield and SIRIS NIRF images of FH and HMC-FH showing the aqueous stability and bright fluorescence of HMC-FH (FIG. 40A). Photostability study of HMC, ICG, and HMC-FH and serial dilution of HMC-FH showing that the SIRIS system has a detection limit for HMC-FH in the low nM range (FIG. 40B).

FIG. 41A-FIG. 41B depicts in accordance with various embodiments of the invention, targeting of HMC-FH to human GBM cells via OATP. HMC-FH internalizes in various GBM cells, fluorescently labeling the cells (FIG. 41A). An OATP inhibitor (Atazanir) inhibits HMC-FH internalization via fluorescent microscopy and flow (FIG. 41B).

FIG. 42A-FIG. 42F depicts in accordance with various embodiments of the invention, HMC-FH accumulates in intracranial human GBM tumors in mice. SIRIS can visualize the distribution of HMC-FH in various organs and specifically in a GBM tumor, resulting in stable fluorescent labeling of the tumor 3 h (FIG. 42A), 24 h (FIG. 42B) or 168 h (FIG. 42C) after HMC-FH i.v. injection. Corresponding time-dependent quantification of HMC-FH organ distribution (FIG. 42D), tumor-to-healthy brain fluorescence ration (FIG. 42E) and blood fluorescence (FIG. 42F).

FIG. 43A-FIG. 43C depicts in accordance with various embodiments of the invention, HMC-FH fluorescently label U87MG GBM tumors in mice facilitating tumor visualization and surgical removal. GMB tumor extraction procedure, visualized and recorded by SIRIS (FIG. 43A). Images of mouse brain with GMB tumors previously injected with HMC-FH, HMC or ICG before and after tumor removal (FIG. 43B). SIRIS fluorescence image of a large GBM tumor, showing strong fluorescence in the tumor and in the area surrounding the “surgical” cavity (FIG. 43C).

FIG. 44A-FIG. 44D depicts in accordance with various embodiments of the invention, Targeting and accumulation of HMC-FH to U87MG GBM tumors in mice via BBB crossing. Microscopic images of a GBM tumor indicates a perfect match between the near infrared fluorescent (NIRF) and the H&E stained images in the tumor section (FIG. 44A) as well as near the tumor border (FIG. 44B). Immunohistopathology of tumor and tumor infiltrate areas indicates that HMC-FH (red signal) associates with the U87MG cells (nesting staining, green signal) (FIG. 44C). However, no association between HMC-FH (red signal) and von Willebrand positive blood vessel is observed, indicating successful BBB crossing (FIG. 44D).

FIG. 45A-FIG. 45C depicts in accordance with various embodiments of the invention, targeting of HMC-FH(PTX) to human GBM cells reduces cell viability via induction of apoptosis. Microscopy images of various GBM cell lines treated with HMC-FH(PTX) show visible changes in cell morphology (FIG. 45A), with reduction in cell viability with estimated IC₅₀ in the low nm range. (FIG. 45B). Flow apoptosis assay showing a significant decrease in viable cells, with a corresponding increase in the population of early and late apoptotic cells (FIG. 45C).

FIG. 46A-FIG. 46D depicts in accordance with various embodiments of the invention, HMC-FH(PTX) reduces the growth of U87MG GBM tumors in mice. Brain MRI images of treated mice (FIG. 46A). Tumor volume measurements by MRI of mice (n=5 per group) (FIG. 46B) Kaplan-Meier curves showing significant increase survival in mice treated with HMC-FH(PTX) (FIG. 46C). Corresponding mice body weight measurements (FIG. 46D).

FIG. 47 depicts in accordance with various embodiments of the invention, Histopathological confirmation of the absent of tumor in the HMC-FH(PTX) treated mice brain during the treatment period. No visible tumor is observed in the brains of the treated mice. In contrast, tumor is observed in the control (PBS).

FIG. 48A-FIG. 48F depicts in accordance with various embodiments of the invention, HMC-FH can target patient derived GBM stem cells, fluorescently labeling those cells and corresponding brain tumor in mice. GBM Stem cell spheroids fluorescently labeled with HMC-FH (FIG. 48A) Corresponding intracranial GBM tumor xenographs showing accumulation of HMC-FH in GBM tumors (FIG. 48B) that correspond to H&E staining of these tumors (FIG. 48C, FIG. 48D). When these cells were incubated with HMC-FH(PTX) or HMC-BFA for 4 days, a disruption of spheroids was observed with an increased in the number of apoptotic cells (FIG. 48E). Further experiments upon 8 days incubation period indicate that HMC-Fh(BFA) greatly reduce the number of viable cells in contrast to HMC-FH or FH(BFA).

FIG. 49 depicts in accordance with various embodiments of the invention, Kaplan-Meier curves showing significant increase survival in mice treated with HMC-FH(BFA).

### DETAILED DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3^{rd} ed., Wiley-Blackwell (Nov. 28, 2012); Brent et al., Current Protocols in Molecular Biology, John Wiley & Sons, Inc. (ringbou ed., 2003); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 July, 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Other features and advantages of the invention will become apparent from the following detailed description, taken in conjunction with the accompanying drawings, which illustrate, by way of example, various features of embodiments of the invention. Indeed, the present invention is in no way limited to the methods and materials described. For convenience, certain terms employed herein, in the specification, examples and appended claims are collected here.

Unless stated otherwise, or implicit from context, the following terms and phrases include the meanings provided below. Unless explicitly stated otherwise, or apparent from context, the terms and phrases below do not exclude the meaning that the term or phrase has acquired in the art to which it pertains. The definitions and terminology used herein are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. It should be understood that this invention is not limited to the particular methodology, protocols, and reagents, etc., described herein and as such can vary. The definitions and terminology used herein are provided to aid in describing particular embodiments, and are not intended to limit the claimed invention.

As used herein the term “comprising” or “comprises” is used in reference to compositions, methods, systems, articles of manufacture, and respective component(s) thereof, that are useful to an embodiment, yet open to the inclusion of unspecified elements, whether useful or not. It will be understood by those within the art that, in general, terms used herein are generally intended as “open” terms (e.g., the term “including” should be interpreted as “including but not limited to,” the term “having” should be interpreted as “having at least,” the term “includes” should be interpreted as “includes but is not limited to,” etc.). Although the open-ended term “comprising,” as a synonym of terms such as including, containing, or having, is used herein to describe and claim the invention, the present invention, or embodiments thereof, may alternatively be described using alternative terms such as “consisting of” or “consisting essentially of”

Unless stated otherwise, the terms “a” and “an” and “the” and similar references used in the context of describing a particular embodiment of the application (especially in the context of claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. The abbreviation, “e.g.” is derived from the Latin exempli gratia, and is used herein to indicate a non-limiting example. Thus, the abbreviation “e.g.” is synonymous with the term “for example.” No language in the specification should be construed as indicating any non-claimed element essential to the practice of the application.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

“Optional” or “optionally” means that the subsequently described circumstance may or may not occur, so that the description includes instances where the circumstance occurs and instances where it does not.

As used herein, the term “substituted” refers to independent replacement of one or more (typically 1, 2, 3, 4, or 5) of the hydrogen atoms on the substituted moiety with substituents independently selected from the group of substituents listed below in the definition for “substituents” or otherwise specified. In general, a non-hydrogen substituent can be any substituent that can be bound to an atom of the given moiety that is specified to be substituted. Examples of substituents include, but are not limited to, acyl, acylamino, acyloxy, aldehyde, alicyclic, aliphatic, alkanesulfonamido, alkanesulfonyl, alkaryl, alkenyl, alkoxy, alkoxycarbonyl, alkyl, alkylamino, alkylcarbanoyl, alkylene, alkylidene, alkylthios, alkynyl, amide, amido, amino, amidine, aminoalkyl, aralkyl, aralkylsulfonamido, arenesulfonamido, arenesulfonyl, aromatic, aryl, arylamino, arylcarbanoyl, aryloxy, azido, carbamoyl, carbonyl, carbonyls including ketones, carboxy, carboxylates, CF₃, cyano (CN), cycloalkyl, cycloalkylene, ester, ether, haloalkyl, halogen, halogen, heteroaryl, heterocyclyl, hydroxy, hydroxyalkyl, imino, iminoketone, ketone, mercapto, nitro, oxaalkyl, oxo, oxoalkyl, phosphoryl (including phosphonate and phosphinate), silyl groups, sulfonamido, sulfonyl (including sulfate, sulfamoyl and sulfonate), thiols, and ureido moieties, each of which may optionally also be substituted or unsubstituted. In some cases, two substituents, together with the carbon(s) to which they are attached to, can form a ring. In some cases, two or more substituents, together with the carbon(s) to which they are attached to, can form one or more rings.

The terms “substituted” and “functionalized” are used interchangeably herein.

The terms “unsubstituted” and “unfunctionalized” are used interchangeably herein.

Substituents may be protected as necessary and any of the protecting groups commonly used in the art may be employed. Non-limiting examples of protecting groups may be found, for example, in Greene and Wuts, Protective Groups in Organic Synthesis, 44^{th}. Ed., Wiley & Sons, 2006.

As used herein, the term “alkyl” means a straight or branched, saturated aliphatic radical having a chain of carbon atoms. Cₓ alkyl and Cₓ-C_{y}alkyl are typically used where X and Y indicate the number of carbon atoms in the chain. For example, C₁-C₆alkyl includes alkyls that have a chain of between 1 and 6 carbons (e.g., methyl, ethyl, propyl, isopropyl, butyl, sec-butyl, isobutyl, tert-butyl, pentyl, neopentyl, hexyl, and the like). Alkyl represented along with another radical (e.g., as in arylalkyl) means a straight or branched, saturated alkyl divalent radical having the number of atoms indicated or when no atoms are indicated means a bond, e.g., (C₆-C₁₀)aryl(C₀-C₃)alkyl includes phenyl, benzyl, phenethyl, 1-phenylethyl 3-phenylpropyl, and the like. Backbone of the alkyl can be optionally inserted with one or more heteroatoms, such as N, O, or S.

In some embodiments, a straight chain or branched chain alkyl has 30 or fewer carbon atoms in its backbone (e.g., C₁-C₃₀ for straight chains, C₃-C₃₀ for branched chains), and in some embodiments 20 or fewer. Likewise, in some embodiments cycloalkyls have from 3-10 carbon atoms in their ring structure, and some embodiments have 5, 6 or 7 carbons in the ring structure. The term “alkyl” (or “lower alkyl”) as used throughout the specification, examples, and claims is intended to include both “unsubstituted alkyls” and “substituted alkyls”, the latter of which refers to alkyl moieties having one or more substituents replacing a hydrogen on one or more carbons of the hydrocarbon backbone.

Unless the number of carbons is otherwise specified, “lower alkyl” as used herein means an alkyl group, as defined above, but having from one to ten carbons, in some embodiments from one to six carbon atoms in its backbone structure. Likewise, “lower alkenyl” and “lower alkynyl” have similar chain lengths. Throughout the application, in some embodiments alkyl groups are lower alkyls. In some embodiments, a substituent designated herein as alkyl is a lower alkyl.

Non-limiting examples of substituents of a substituted alkyl can include halogen, hydroxy, nitro, thiols, amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like.

As used herein, the term “alkenyl” refers to unsaturated straight-chain, branched-chain or cyclic hydrocarbon radicals having at least one carbon-carbon double bond. Cₓ alkenyl and Cₓ-C_{y}alkenyl are typically used where X and Y indicate the number of carbon atoms in the chain. For example, C₂-C₆alkenyl includes alkenyls that have a chain of between 2 and 6 carbons and at least one double bond, e.g., vinyl, allyl, propenyl, isopropenyl, 1-butenyl, 2-butenyl, 3-butenyl, 2-methylallyl, 1-hexenyl, 2-hexenyl, 3-hexenyl, and the like). Alkenyl represented along with another radical (e.g., as in arylalkenyl) means a straight or branched, alkenyl divalent radical having the number of atoms indicated. Backbone of the alkenyl can be optionally inserted with one or more heteroatoms, such as N, O, or S.

As used herein, the term “alkynyl” refers to unsaturated hydrocarbon radicals having at least one carbon-carbon triple bond. Cₓ alkynyl and Cₓ-C_{y}alkynyl are typically used where X and Y indicate the number of carbon atoms in the chain. For example, C₂-C₆alkynyl includes alkynls that have a chain of between 2 and 6 carbons and at least one triple bond, e.g., ethynyl, 1-propynyl, 2-propynyl, 1-butynyl, isopentynyl, 1,3-hexa-diyn-yl, n-hexynyl, 3-pentynyl, 1-hexen-3-ynyl and the like. Alkynyl represented along with another radical (e.g., as in arylalkynyl) means a straight or branched, alkynyl divalent radical having the number of atoms indicated. Backbone of the alkynyl can be optionally inserted with one or more heteroatoms, such as N, O, or S.

The terms “alkylene,” “alkenylene,” and “alkynylene” refer to divalent alkyl, alkenyl, and alkynyl” radicals. Prefixes Cₓ and Cₓ-C_{y} are typically used where X and Y indicate the number of carbon atoms in the chain. For example, C₁-C₆alkylene includes methylene, (—CH₂—), ethylene (—CH₂CH₂—), trimethylene (—CH₂CH₂CH₂—), tetramethylene (—CH₂CH₂CH₂CH₂—), 2-methyltetramethylene (—CH₂CH(CH₃)CH₂CH₂—), pentamethylene (—CH₂CH₂CH₂CH₂CH₂—) and the like).

As used herein, the term “alkylidene” means a straight or branched unsaturated, aliphatic, divalent radical having a general formula ═CRₐR_{b}. Non-limiting examples of Rₐ and R_{b} are each independently hydrogen, alkyl, substituted alkyl, alkenyl, or substituted alkenyl. Cₓ alkylidene and Cₓ-C_{y}alkylidene are typically used where X and Y indicate the number of carbon atoms in the chain. For example, C₂-C₆alkylidene includes methylidene (═CH₂), ethylidene (═CHCH₃), isopropylidene (═C(CH₃)₂), propylidene (═CHCH₂CH₃), allylidene (═CH—CH═CH₂), and the like).

The term “heteroalkyl”, as used herein, refers to straight or branched chain, or cyclic carbon-containing radicals, or combinations thereof, containing at least one heteroatom. Suitable heteroatoms include, but are not limited to, O, N, Si, P, Se, B, and S, wherein the phosphorous and sulfur atoms are optionally oxidized, and the nitrogen heteroatom is optionally quaternized. Heteroalkyls can be substituted as defined above for alkyl groups.

As used herein, the term “halogen” or “halo” refers to an atom selected from fluorine, chlorine, bromine and iodine. The term “halogen radioisotope” or “halo isotope” refers to a radionuclide of an atom selected from fluorine, chlorine, bromine and iodine.

A “halogen-substituted moiety” or “halo-substituted moiety”, as an isolated group or part of a larger group, means an aliphatic, alicyclic, or aromatic moiety, as described herein, substituted by one or more “halo” atoms, as such terms are defined in this application. For example, halo-substituted alkyl includes haloalkyl, dihaloalkyl, trihaloalkyl, perhaloalkyl and the like (e.g. halosubstituted (C₁-C₃)alkyl includes chloromethyl, dichloromethyl, difluoromethyl, trifluoromethyl (—CF₃), 2,2,2-trifluoroethyl, perfluoroethyl, 2,2,2-trifluoro-1,1-dichloroethyl, and the like).

The term “aryl” refers to monocyclic, bicyclic, or tricyclic fused aromatic ring system. Cₓ aryl and Cₓ-C_{y}aryl are typically used where X and Y indicate the number of carbon atoms in the ring system. For example, C₆-C₁₂ aryl includes aryls that have 6 to 12 carbon atoms in the ring system. Exemplary aryl groups include, but are not limited to, pyridinyl, pyrimidinyl, furanyl, thienyl, imidazolyl, thiazolyl, pyrazolyl, pyridazinyl, pyrazinyl, triazinyl, tetrazolyl, indolyl, benzyl, phenyl, naphthyl, anthracenyl, azulenyl, fluorenyl, indanyl, indenyl, naphthyl, phenyl, tetrahydronaphthyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH carbazolyl, carbolinyl, chromanyl, chromenyl, cinnolinyl, decahydroquinolinyl, 2H,6H-1,5,2-dithiazinyl, dihydrofuro[2,3b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl, and the like. In some embodiments, 1, 2, 3, or 4 hydrogen atoms of each ring can be substituted by a substituent.

The term “heteroaryl” refers to an aromatic 5-8 membered monocyclic, 8-12 membered fused bicyclic, or 11-14 membered fused tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively. Cₓ heteroaryl and Cₓ-C_{y}heteroaryl are typically used where X and Y indicate the number of carbon atoms in the ring system. For example, C₄-C₉ heteroaryl includes heteroaryls that have 4 to 9 carbon atoms in the ring system. Heteroaryls include, but are not limited to, those derived from benzo[b]furan, benzo[b] thiophene, benzimidazole, imidazo[4,5-c]pyridine, quinazoline, thieno[2,3-c]pyridine, thieno[3,2-b]pyridine, thieno[2,3-b]pyridine, indolizine, imidazo[1,2a]pyridine, quinoline, isoquinoline, phthalazine, quinoxaline, naphthyridine, quinolizine, indole, isoindole, indazole, indoline, benzoxazole, benzopyrazole, benzothiazole, imidazo[1,5-a]pyridine, pyrazolo[1,5-a]pyridine, imidazo[1,2-a]pyrimidine, imidazo[1,2-c]pyrimidine, imidazo[1,5-a]pyrimidine, imidazo[1,5-c]pyrimidine, pyrrolo[2,3-b]pyridine, pyrrolo[2,3cjpyridine, pyrrolo[3,2-c]pyridine, pyrrolo[3,2-b]pyridine, pyrrolo[2,3-d]pyrimidine, pyrrolo[3,2-d]pyrimidine, pyrrolo[2,3-b]pyrazine, pyrazolo[1,5-a]pyridine, pyrrolo[1,2-b]pyridazine, pyrrolo[1,2-c]pyrimidine, pyrrolo[1,2-a]pyrimidine, pyrrolo[1,2-a]pyrazine, triazo[1,5-a]pyridine, pteridine, purine, carbazole, acridine, phenazine, phenothiazene, phenoxazine, 1,2-dihydropyrrolo[3,2,1-hi]indole, indolizine, pyrido[1,2-a]indole, 2 (1H)-pyridinone, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxepanyl, oxetanyl, oxindolyl, pyrimidinyl, phenanthridinyl, phenanthrolinyl, phenazinyl, phenothiazinyl, phenoxathinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, 2H-pyrrolyl, pyrrolyl, quinazolinyl, quinolinyl, 4H-quinolizinyl, quinoxalinyl, quinuclidinyl, tetrahydrofuranyl, tetrahydroisoquinolinyl, tetrahydropyranyl, tetrahydroquinolinyl, tetrazolyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. Some exemplary heteroaryl groups include, but are not limited to, pyridyl, furyl or furanyl, imidazolyl, benzimidazolyl, pyrimidinyl, thiophenyl or thienyl, pyridazinyl, pyrazinyl, quinolinyl, indolyl, thiazolyl, naphthyridinyl, 2-amino-4-oxo-3,4-dihydropteridin-6-yl, tetrahydroisoquinolinyl, and the like. In some embodiments, 1, 2, 3, or 4 hydrogen atoms of each ring may be substituted by a substituent.

The term “cyclyl” or “cycloalkyl” refers to saturated and partially unsaturated cyclic hydrocarbon groups having 3 to 12 carbons, for example, 3 to 8 carbons, and, for example, 3 to 6 carbons. Cₓcyclyl and Cₓ-C_{y}cycyl are typically used where X and Y indicate the number of carbon atoms in the ring system. For example, C₃-C₈ cyclyl includes cyclyls that have 3 to 8 carbon atoms in the ring system. The cycloalkyl group additionally can be optionally substituted, e.g., with 1, 2, 3, or 4 substituents. C₃-C₁₀cyclyl includes cyclopropyl, cyclobutyl, cyclopentyl, cyclohexyl, cyclohexenyl, 2,5-cyclohexadienyl, cycloheptyl, cyclooctyl, bicyclo[2.2.2]octyl, adamantan-1-yl, decahydronaphthyl, oxocyclohexyl, dioxocyclohexyl, thiocyclohexyl, 2-oxobicyclo [2.2.1]hept-1-yl, and the like.

Aryl and heteroaryls can be optionally substituted with one or more substituents at one or more positions, for example, halogen, alkyl, aralkyl, alkenyl, alkynyl, cycloalkyl, hydroxyl, amino, nitro, sulfhydryl, imino, amido, phosphate, phosphonate, phosphinate, carbonyl, carboxyl, silyl, ether, alkylthio, sulfonyl, ketone, aldehyde, ester, a heterocyclyl, an aromatic or heteroaromatic moiety, —CF₃, —CN, or the like.

The term “heterocyclyl” refers to a nonaromatic 4-8 membered monocyclic, 8-12 membered bicyclic, or 11-14 membered tricyclic ring system having 1-3 heteroatoms if monocyclic, 1-6 heteroatoms if bicyclic, or 1-9 heteroatoms if tricyclic, said heteroatoms selected from O, N, or S (e.g., carbon atoms and 1-3, 1-6, or 1-9 heteroatoms of N, O, or S if monocyclic, bicyclic, or tricyclic, respectively). Cₓheterocyclyl and Cₓ-C_{y}heterocyclyl are typically used where X and Y indicate the number of carbon atoms in the ring system. For example, C₄-C₉ heterocyclyl includes heterocyclyls that have 4-9 carbon atoms in the ring system. In some embodiments, 1, 2 or 3 hydrogen atoms of each ring can be substituted by a substituent. Exemplary heterocyclyl groups include, but are not limited to piperazinyl, pyrrolidinyl, dioxanyl, morpholinyl, tetrahydrofuranyl, piperidyl, 4-morpholyl, 4-piperazinyl, pyrrolidinyl, perhydropyrrolizinyl, 1,4-diazaperhydroepinyl, 1,3-dioxanyl, 1,4-dioxanyl and the like.

The terms “bicyclic” and “tricyclic” refers to fused, bridged, or joined by a single bond polycyclic ring assemblies.

The term “cyclylalkylene” means a divalent aryl, heteroaryl, cyclyl, or heterocyclyl.

As used herein, the term “fused ring” refers to a ring that is bonded to another ring to form a compound having a bicyclic structure when the ring atoms that are common to both rings are directly bound to each other. Non-exclusive examples of common fused rings include decalin, naphthalene, anthracene, phenanthrene, indole, furan, benzofuran, quinoline, and the like. Compounds having fused ring systems can be saturated, partially saturated, cyclyl, heterocyclyl, aromatics, heteroaromatics, and the like.

The term “carbocyclyl” as used either alone or in combination with another radical, means a mono- bi- or tricyclic ring structure consisting of 3 to 14 carbon atoms. In some embodiments, one or more of the hydrogen atoms of a carbocyclyl may be optionally substituted by a substituent.

The term “carbocycle” refers to fully saturated ring systems and saturated ring systems and partially saturated ring systems and aromatic ring systems and non-aromatic ring systems and unsaturated ring systems and partially unsaturated ring systems. The term “carbocycle” encompasses monocyclic, bicyclic, polycyclic, spirocyclic, fused, bridged, or linked ring systems. In some embodiments, one or more of the hydrogen atoms of a carbocycle may be optionally substituted by a substituent. In some embodiments the carbocycle optionally comprises one or more heteroatoms. In some embodiments the heteroatoms are selected from N, O, S, or P.

The terms “cyclic” “cyclic group” and “ring” or “rings” means carbocycles, which can be fully saturated, saturated, partially saturated, unsaturated, partially unsaturated non-aromatic or aromatic that may or may not be substituted and which optionally can comprise one or more heteroatoms. In some embodiments the heteroatoms are selected from N, O, S, or P. In some embodiments, one or more of the hydrogen atoms of a ring may be optionally substituted by a substituent. In some embodiments, the ring or rings may be monocyclic, bicyclic, polycyclic, spirocyclic, fused, bridged, or linked.

The term “spiro-cycloalkyl” (spiro) means spirocyclic rings where the ring is linked to the molecule through a carbon atom, and wherein the resulting carbocycle is formed by alkylene groups. The term “spiro-C₃-C₈-cycloalkyl” (spiro) means 3-8 membered, spirocyclic rings where the ring is linked to the molecule through a carbon atom, and wherein the resulting 3-8 membered carbocycle is formed by alkylene groups with 2 to 7 carbon atoms. The term “spiro-C₅-cycloalkyl” (spiro) means 5 membered, spirocyclic rings where the ring is linked to the molecule through a carbon atom, wherein the resulting 5 membered carbocycle is formed by an alkylene group with 4 carbon atoms.

The term “spiro-cycloalkenyl” (spiro) means spirocyclic rings where the ring is linked to the molecule through a carbon atom, and wherein the resulting carbocycle is formed by alkenylene groups. The term “spiro-C₃-C₈-cycloalkenyl” (spiro) means 3-8 membered, spirocyclic rings where the ring is linked to the molecule through a carbon atom, wherein the resulting 3-8 membered carbocycle is formed by alkenylene groups with 2 to 7 carbon atoms. The term “spiro-C₅-cycloalkenyl” (spiro) means 5 membered, spirocyclic rings where the ring is linked to the molecule through a carbon atom, wherein the resulting 5 membered carbocycle is formed by alkenylene groups with 4 carbon atoms.

The term “spiro-heterocyclyl” (spiro) means saturated or unsaturated spirocyclic rings, which may contain one or more heteroatoms, where the ring may be linked to the molecule through a carbon atom or optionally through a nitrogen atom, if a nitrogen atom is present. In some embodiments, the heteroatom is selected from O, N, S, or P. In some embodiments, the heteroatom is O, S, or N. The term “spiro-C₃-C₈-heterocyclyl” (spiro) means 3-8 membered, saturated or unsaturated, spirocyclic rings which may contain one or more heteroatoms, where the ring may be linked to the molecule through a carbon atom or optionally through a nitrogen atom, if a nitrogen atom is present. In some embodiments, the heteroatom is selected from O, N, S, or P. In some embodiments, the heteroatom is O, S, or N. The term “spiro-C₅-heterocyclyl” (spiro) means 5 membered, saturated or unsaturated, spirocyclic rings which may contain one or more heteroatoms, where the ring may be linked to the molecule through a carbon atom or optionally through a nitrogen atom, if a nitrogen atom is present. In some embodiments, the heteroatom is selected from O, N, S, or P. In some embodiments, the heteroatom is O, S, or N.

In some embodiments, one or more of the hydrogen atoms of a spirocyclic ring may be optionally substituted by a substituent. In some embodiments, one or more hydrogen atoms of a spiro-cycloalkyl may be optionally substituted by a substituent. In some embodiments, one or more hydrogen atoms of a spiro-C₃-C₈-cycloalkyl may be optionally substituted by a substituent. In some embodiments, one or more hydrogen atoms of a spiro-C₅-cycloalkyl may be optionally substituted by a substituent. In some embodiments, one or more hydrogen atoms of a spiro-cycloalkenyl may be optionally substituted by a substituent. In some embodiments, one or more hydrogen atoms of a spiro-C₃-C₈-cycloalkenyl may be optionally substituted by a substituent. In some embodiments, one or more hydrogen atoms of a spiro-C₅-cycloalkenyl may be optionally substituted by a substituent. In some embodiments, one or more hydrogen atoms of a spiro-heterocycyl may be optionally substituted by a substituent. In some embodiments, one or more hydrogen atoms of a spiro-C₃-C₈-heterocycyl may be optionally substituted by a substituent. In some embodiments, one or more hydrogen atoms of a spiro-C₅-heterocycyl may be optionally substituted by a substituent.

As used herein, the term “carbonyl” means the radical —C(O)—. It is noted that the carbonyl radical can be further substituted with a variety of substituents to form different carbonyl groups including acids, acid halides, amides, esters, ketones, and the like.

The term “carboxy” means the radical —C(O)O—. It is noted that compounds described herein containing carboxy moieties can include protected derivatives thereof, i.e., where the oxygen is substituted with a protecting group. Suitable protecting groups for carboxy moieties include benzyl, tert-butyl, and the like. The term “carboxyl” means —COOH.

The term “cyano” means the radical —CN.

The term, “heteroatom” refers to an atom that is not a carbon atom. Particular examples of heteroatoms include, but are not limited to nitrogen, oxygen, sulfur and halogens. A “heteroatom moiety” includes a moiety where the atom by which the moiety is attached is not a carbon. Examples of heteroatom moieties include —N═, —NR^{N}—, —N⁺(O⁻)═, —O—, —S— or —S(O)₂—, —OS(O)₂—, and —SS—, wherein R^{N} is H or a further substituent.

The term “hydroxy” means the radical —OH.

The term “imine derivative” means a derivative comprising the moiety —C(NR)—, wherein R comprises a hydrogen or carbon atom alpha to the nitrogen.

The term “nitro” means the radical —NO₂.

An “oxaaliphatic,” “oxaalicyclic”, or “oxaaromatic” mean an aliphatic, alicyclic, or aromatic, as defined herein, except where one or more oxygen atoms (—O—) are positioned between carbon atoms of the aliphatic, alicyclic, or aromatic respectively.

An “oxoaliphatic,” “oxoalicyclic”, or “oxoaromatic” means an aliphatic, alicyclic, or aromatic, as defined herein, substituted with a carbonyl group. The carbonyl group can be an aldehyde, ketone, ester, amide, acid, or acid halide.

As used herein, the term “oxo” means the substituent ═O.

As used herein, the term, “aromatic” means a moiety wherein the constituent atoms make up an unsaturated ring system, all atoms in the ring system are sp² hybridized and the total number of pi electrons is equal to 4n+2. An aromatic ring can be such that the ring atoms are only carbon atoms (e.g., aryl) or can include carbon and non-carbon atoms (e.g., heteroaryl).

The terms “alkoxyl” or “alkoxy” as used herein refers to an alkyl group, as defined above, having an oxygen radical attached thereto. Representative alkoxyl groups include methoxy, ethoxy, propyloxy, tert-butoxy, n-propyloxy, iso-propyloxy, n-butyloxy, iso-butyloxy, and the like. An “ether” is two hydrocarbons covalently linked by an oxygen. Accordingly, the substituent of an alkyl that renders that alkyl an ether is or resembles an alkoxyl, such as can be represented by one of —O-alkyl, —O-alkenyl, and —O-alkynyl. Aroxy can be represented by —O-aryl or O-heteroaryl, wherein aryl and heteroaryl are as defined below. The alkoxy and aroxy groups can be substituted as described above for alkyl.

The term “aralkyl”, as used herein, refers to an alkyl group substituted with an aryl group (e.g., an aromatic or heteroaromatic group).

The term “alkylthio” refers to an alkyl group, as defined above, having a sulfur radical attached thereto. In some embodiments, the “alkylthio” moiety is represented by one of —S-alkyl, —S-alkenyl, and —S-alkynyl. Representative alkylthio groups include methylthio, ethylthio, and the like. The term “alkylthio” also encompasses cycloalkyl groups, alkene and cycloalkene groups, and alkyne groups. “Arylthio” refers to aryl or heteroaryl groups.

The term “sulfinyl” means the radical —SO—. It is noted that the sulfinyl radical can be further substituted with a variety of substituents to form different sulfinyl groups including sulfinic acids, sulfinamides, sulfinyl esters, sulfoxides, and the like.

The term “sulfonyl” means the radical —SO₂—. It is noted that the sulfonyl radical can be further substituted with a variety of substituents to form different sulfonyl groups including sulfonic acids (—SO₃H), sulfonamides, sulfonate esters, sulfones, and the like.

The term “thiocarbonyl” means the radical —C(S)—. It is noted that the thiocarbonyl radical can be further substituted with a variety of substituents to form different thiocarbonyl groups including thioacids, thioamides, thioesters, thioketones, and the like.

As used herein, the term “amino” means —NH₂. The term “alkylamino” means a nitrogen moiety having at least one straight or branched unsaturated aliphatic, cyclyl, or heterocyclyl radicals attached to the nitrogen. For example, representative amino groups include —NH₂, —NHCH₃, —N(CH₃)₂, —NH(C₁-C₁₀alkyl), N(C₁-C₁₀alkyl)₂, and the like. The term “alkylamino” includes “alkenylamino,” “alkynylamino,” “cyclylamino,” and “heterocyclylamino.” The term “arylamino” means a nitrogen moiety having at least one aryl radical attached to the nitrogen. For example —NHaryl, and —N(aryl)₂. The term “heteroarylamino” means a nitrogen moiety having at least one heteroaryl radical attached to the nitrogen. For example NHheteroaryl, and —N(heteroaryl)₂. Optionally, two substituents together with the nitrogen can also form a ring. Unless indicated otherwise, the compounds described herein containing amino moieties can include protected derivatives thereof. Suitable protecting groups for amino moieties include acetyl, tertbutoxycarbonyl, benzyloxycarbonyl, and the like.

The term “aminoalkyl” means an alkyl, alkenyl, and alkynyl as defined above, except where one or more substituted or unsubstituted nitrogen atoms (—N—) are positioned between carbon atoms of the alkyl, alkenyl, or alkynyl. For example, an (C₂-C₆) aminoalkyl refers to a chain comprising between 2 and 6 carbons and one or more nitrogen atoms positioned between the carbon atoms.

The term “alkoxyalkoxy” means —O-(alkyl)-O-(alkyl), such as —OCH₂CH₂OCH₃, and the like.

The term “alkoxycarbonyl” means —C(O)O-(alkyl), such as —C(═O)OCH₃, —C(═O)OCH₂CH₃, and the like.

The term “alkoxyalkyl” means -(alkyl)-O-(alkyl), such as —CH₂OCH₃, —CH₂OCH₂CH₃, and the like.

The term “aryloxy” means —O-(aryl), such as —O-phenyl, —O-pyridinyl, and the like.

The term “arylalkyl” means -(alkyl)-(aryl), such as benzyl (i.e., —CH₂phenyl), —CH₂-pyrindinyl, and the like.

The term “arylalkyloxy” means —O-(alkyl)-(aryl), such as —O-benzyl, —O—CH₂-pyridinyl, and the like.

The term “cycloalkyloxy” means —O-(cycloalkyl), such as —O-cyclohexyl, and the like.

The term “cycloalkylalkyloxy” means —O-(alkyl)-(cycloalkyl, such as —OCH₂cyclohexyl, and the like.

The term “aminoalkoxy” means —O-(alkyl)-NH₂, such as —OCH₂NH₂, —OCH₂CH₂NH₂, and the like.

The term “mono- or di-alkylamino” means —NH(alkyl) or —N(alkyl)(alkyl), respectively, such as —NHCH₃, —N(CH₃)₂, and the like.

The term “mono- or di-alkylaminoalkoxy” means —O-(alkyl)-NH(alkyl) or —O-(alkyl)-N(alkyl)(alkyl), respectively, such as —OCH₂NHCH₃, —OCH₂CH₂N(CH₃)₂, and the like.

The term “arylamino” means —NH(aryl), such as —NH-phenyl, —NH-pyridinyl, and the like.

The term “arylalkylamino” means —NH-(alkyl)-(aryl), such as —NH-benzyl, —NHCH₂-pyridinyl, and the like.

The term “alkylamino” means —NH(alkyl), such as —NHCH₃, —NHCH₂CH₃, and the like.

The term “cycloalkylamino” means —NH-(cycloalkyl), such as —NH-cyclohexyl, and the like.

The term “cycloalkylalkylamino” —NH-(alkyl)-(cycloalkyl), such as —NHCH₂-cyclohexyl, and the like.

The term “sulfonato” means —SO₃.

The term “PEGyl” refers to a polyethylene chain with repeated moiety of (—CH₂—CH₂—O—)ₙ. n is ranging from 2 to 20. The remote end of the PEG may be optionally functionalized with amino, carboxylate, sulfonate, alkyne, sulfohydryl, hydroxyl, or any other functional group.

“Electron withdrawing group” or EWG refers to functional groups that remove electron density from the ring by making it less nucleophilic. This class can be recognized by the atom adjacent to the π system having several bonds to more electronegative atoms or the presence of a formal charge. Non-limiting examples of these groups include halogens, aldehydes, ketones, esters, carboxylic acids, acid chlorides, nitriles, nitrosos, and sulfonic acids.

“Electron donating group” or EDG refers to functional groups that add electron density to the ring by making it more nucleophilic. This class can be recognized by lone pairs on the atom adjacent to the π system. Non-limiting examples of these groups include alkyl, alkenyl, alkynyl, amides, ethers, alkoxides, alcohols, and amines.

Some commonly used abbreviations are: Me is methyl (—CH₃), Et is ethyl (CH₂—CH₃), Ph is phenyl (—C₆H₅), t-Bu is tert-butyl (—C(CH₃)₃, n-Pr is n-propyl (—CH₂—CH₂—CH₃), Bn is benzyl (—CH₂—C₆H₅).

It is noted in regard to all of the definitions provided herein that the definitions should be interpreted as being open ended in the sense that further substituents beyond those specified may be included. Hence, a C₁ alkyl indicates that there is one carbon atom but does not indicate what are the substituents on the carbon atom. Hence, a C₁ alkyl comprises methyl (i.e., —CH₃) as well as —CRₐR_{b}R_{c} where Rₐ, R_{b}, and R_{c} can each independently be hydrogen or any other substituent where the atom alpha to the carbon is a heteroatom or cyano. Hence, CF₃, CH₂OH and CH₂CN are all C₁ alkyls.

As used herein, the terms “heptamethine cyanine (HMC)”, “heptamethine carbocyanine (HMC)” and “HMC” have the same meaning and refer to the following compound:

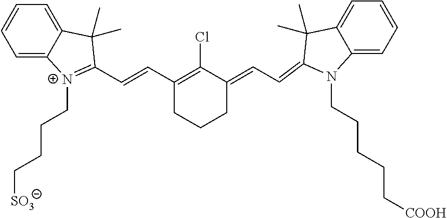

Unless otherwise stated, structures depicted herein are meant to include compounds which differ only in the presence of one or more isotopically enriched atoms. For example, compounds having the present structure except for the replacement of a hydrogen atom by a deuterium or tritium, or the replacement of a carbon atom by a ¹³C- or ¹⁴C-enriched carbon are within the scope of the invention.

Synthetic Preparation. In various embodiments, compounds, compositions, formulations, articles of manufacture, reagents, products, etc. (e.g., compositions, polymers, copolymers, nanoparticles, etc.) of the present invention as disclosed herein may be synthesized using any synthetic method available to one of skill in the art. In various embodiments, the compounds, compositions, formulations, articles of manufacture, reagents, products, etc. (e.g., compositions, polymers, copolymers, nanoparticles, etc.) of the present invention disclosed herein can be prepared in a variety of ways known to one skilled in the art of organic synthesis, inorganic synthesis, and/or organometallic synthesis and in analogy with the exemplary compounds, compositions, formulations, articles of manufacture, reagents, products, etc. whose synthesis is described herein. The starting materials used in preparing these compounds, compositions, formulations, articles of manufacture, reagents, products, etc. may be commercially available or prepared by known methods. Preparation of compounds, can involve the protection and deprotection of various chemical groups. The need for protection and deprotection, and the selection of appropriate protecting groups can be readily determined by one skilled in the art. The chemistry of protecting groups can be found, for example, in Greene and Wuts, Protective Groups in Organic Synthesis, 44th. Ed., Wiley & Sons, 2006, which is incorporated herein by reference in its entirety.

Non-limiting examples of synthetic methods used to prepare various embodiments of compounds, compositions, formulations, articles of manufacture, reagents, products, etc. (e.g., compositions, polymers, copolymers, nanoparticles, etc.) of the invention are disclosed in the Examples section herein. The reactions of the processes described herein can be carried out in suitable solvents which can be readily selected by one of skill in the art of organic synthesis, inorganic synthesis, and/or organometallic synthesis. Suitable solvents can be substantially nonreactive with the starting materials (reactants), the intermediates, or products at the temperatures at which the reactions are carried out, i.e., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected.

As used herein, the terms “treat,” “treatment,” “treating,” or “amelioration” when used in reference to a symptom, disease, disorder, or disease condition, refer to both therapeutic treatment and prophylactic or preventative measures, wherein the object is to reverse, alleviate, ameliorate, inhibit, lessen, slow down or stop the progression or severity of a symptom, disease condition, disease, or disorder. The term “treating” includes reducing or alleviating at least one adverse effect or symptom of a disease condition, disease, or disorder. Treatment is generally “effective” if one or more symptoms or clinical markers are reduced. Alternatively, treatment is “effective” if the progression of a symptom, disease, disorder, disease condition is reduced or halted. That is, “treatment” includes not just the improvement of symptoms or markers, but also a cessation or at least slowing of progress or worsening of symptoms that would be expected in the absence of treatment. Also, “treatment” may mean to pursue or obtain beneficial results, or lower the chances of the individual developing the disease condition, disease, or disorder even if the treatment is ultimately unsuccessful. Those in need of treatment include those already with the symptom, disease condition, disease, or disorder as well as those prone to have the symptom, disease condition, disease, or disorder, or those in whom the symptom, disease condition, disease, or disorder is to be prevented. Treatment also includes a decrease in mortality or an increase in the lifespan of a subject as compared to one not receiving the treatment.

The term “preventative treatment” means maintaining or improving a healthy state or non-diseased state of a healthy subject or subject that does not have a symptom, disease, disorder, or disease condition. The term “preventative treatment” also means to prevent or to slow the appearance of symptoms associated with a disease condition, disease, or disorder. The term “preventative treatment” also means to prevent or slow a subject from obtaining a symptom, disease condition, disease, or disorder.

Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. The term “treatment” of a disease condition, disease, or disorder also includes providing relief from the symptoms or side-effects of the disease, disorder, or disease condition (including palliative treatment). Those in need of treatment include those already with the disease condition, disease, or disorder as well as those prone to have the disease condition, disease, or disorder or those in whom the disease condition, disease, or disorder is to be prevented.

“Beneficial results” or “desired results” may include, but are in no way limited to, lessening or alleviating the severity of the symptom, disease, disorder, or disease condition; preventing the symptom, disease, disorder, or disease condition from worsening; curing the symptom, disease, disorder, or disease condition; preventing the symptom, disease, disorder, or disease condition from developing; lowering the chances of a patient developing the symptom, disease, disorder, or disease condition; decreasing morbidity and mortality; and prolonging a patient's life or life expectancy. As non-limiting examples, “beneficial results” or “desired results” may be alleviation of one or more symptom(s); diminishment of extent of the deficit; stabilized (i.e., not worsening) state of a symptom, disease, disorder, or disease condition; delay or slowing of a symptom, disease, disorder, or disease condition; and amelioration or palliation of symptoms associated with a disease, disorder, or disease condition.

As used herein, the term “administering,” refers to the placement of a compound or agent (e.g., a nanoparticle of the present invention, drug, probe, or pharmaceutical composition) or a treatment as disclosed herein into a subject by a method or route which results in at least partial localization of the compound, agent or treatment at a desired site. “Route of administration” may refer to any administration pathway known in the art, including but not limited to aerosol, nasal, via inhalation, oral, anal, intra-anal, pen-anal, transmucosal, transdermal, parenteral, enteral, topical or local. “Parenteral” refers to a route of administration that is generally associated with injection, including intracranial, intraventricular, intrathecal, epidural, intradural, intraorbital, infusion, intracapsular, intracardiac, intradermal, intramuscular, intraperitoneal, intrapulmonary, intraspinal, intrasternal, intrathecal, intrauterine, intravascular, intravenous, intraarterial, subarachnoid, subcapsular, subcutaneous, transmucosal, or transtracheal. Via the parenteral route, the compositions may be in the form of solutions or suspensions for infusion or for injection, or as lyophilized powders. Via the enteral route, the compound, agent, or treatment can be in the form of tablets, gel capsules, sugar-coated tablets, syrups, suspensions, solutions, powders, granules, emulsions, microspheres or nanospheres or lipid vesicles or polymer vesicles allowing controlled release. Via the topical route, the compound, agent or treatment can be in the form of aerosol, lotion, cream, gel, ointment, suspensions, solutions or emulsions. In accordance with the present invention, “administering” can be self-administering. For example, it is considered as “administering” that a subject consumes a composition, compound, agent or treatment as disclosed herein. (e.g., nanoparticle of the present invention, drug, probe, or pharmaceutical composition).

As used herein, an “effective amount” is that amount effective to bring about the physiological change desired in the subject or sample to which a compound or agent (e.g., nanoparticle of the present invention, drug, probe, or pharmaceutical composition) is administered. The term “therapeutically effective amount” as used herein, means that amount of a compound or agent (e.g., nanoparticle of the present invention, drug, probe, or pharmaceutical composition), alone or in combination, or in combination with another compound or agent according to an embodiment of the invention, that elicits the biological or medicinal response in a subject or sample that is being sought by a researcher, veterinarian, medical doctor, or other clinician, which includes alleviation of the symptoms of the disease, disorder, or disease condition being treated. For example, if the drug is a therapeutic agent, an effective amount of the drug is that amount sufficient to treat a pathological condition (e.g., a disease, disorder, or disease condition) in the subject or sample to which the drug is administered. For example, in the case of cancer, the therapeutically effective amount of the drug may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve, to some extent, one or more of the symptoms associated with the cancer. To the extent the therapeutic agent may prevent growth and/or kill existing cancer cells, it may be cytostatic and/or cytotoxic. For cancer therapy, efficacy can, for example, be measured by assessing the time to disease progression (TTP) and/or determining the response rate (RR).

“Diagnostic” means identifying the presence or nature of a symptom, disease condition, disease, or disorder and includes identifying patients who are at risk of developing a specific disease condition, disease, or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a disease condition, disease, or disorder it suffices if the method provides a positive indication that aids in diagnosis.

The terms “detection”, “detecting” and the like, may be used in the context of detecting a nanoparticle of the present invention bound to a tissue (e.g., a tissue, a cell, a cancerous tissue, cancer tissue, cancer cell, tumor, tumor cell, or tumor tissue). In some embodiments, the terms “detection”, “detecting” and the like, may be used in the context of detecting a disease condition, detecting a disease, or detecting a disorder (e.g. when positive assay results are obtained).

The term “diagnosis,” or “dx,” refers to the identification of the nature and cause of a certain phenomenon. As used herein, a diagnosis typically refers to a medical diagnosis, which is the process of determining which disease, disorder, or disease condition explains a symptoms and signs. A diagnostic procedure, often a diagnostic test or assay, can be used to provide a diagnosis. A diagnosis can comprise detecting the presence of a disease, disorder, or disease condition or the risk of getting a disease, disorder, or disease condition.

The term “prognosis,” or “px,” as used herein refers to predicting the likely outcome of a current standing. For example, a prognosis can include the expected duration and course of a symptom, disease, disorder, or disease condition, such as progressive decline or expected recovery.

The term “theranosis,” or “tx” as used herein refers to a diagnosis or prognosis used in the context of a medical treatment. For example, theranostics can include diagnostic testing used for selecting appropriate and optimal therapies (or the inverse) based on the context of genetic content or other molecular or cellular analysis. Theranostics includes pharmacogenomics, personalized and precision medicine.

As used herein, a “subject” means a human or animal. For example, the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, and canine species, e.g., dog, fox, wolf. The terms, “patient”, “individual” and “subject” are used interchangeably herein. In an embodiment, the subject is mammal. The mammal can be a human, non-human primate, mouse, rat, dog, cat, horse, or cow, but are not limited to these examples. In addition, the methods described herein can be used to treat domesticated animals and/or pets. In some embodiments, the subject is a human.

The terms “subject”, “patient” or “individual” generally refer to a human, although the methods of the invention are not limited to humans, and should be useful in other animals (e.g. birds, reptiles, amphibians, mammals), particularly in mammals, since albumin is homologous among species.

A subject can be one who has been previously diagnosed with or identified as suffering from or having a disease, disorder, or disease condition in need of treatment or one or more complications related to the disease, disorder, or disease condition, and optionally, have already undergone treatment for the disease, disorder, or disease condition, or the one or more complications related to the disease, disorder, or disease condition. Alternatively, a subject can also be one who has not been previously diagnosed as having a disease, disorder, or disease condition, or one or more complications related to the disease, disorder, or disease condition. For example, a subject can be one who exhibits one or more risk factors for a disease, disorder, or disease condition or one or more complications related to the disease, disorder, or disease condition, or a subject who does not exhibit risk factors. For example, a subject can be one who exhibits one or more symptoms for a disease, disorder, or disease condition, or one or more complications related to the disease, disorder, or disease condition, or a subject who does not exhibit symptoms. A “subject in need” of diagnosis or treatment for a particular disease, disorder, or disease condition, can be a subject suspected of having that disease, disorder, disease condition, diagnosed as having that disease, disorder, or disease condition, already treated or being treated for that disease, disorder, or disease condition, not treated for that disease, disorder, or disease condition, or at risk of developing that disease, disorder, or disease condition.

In some embodiments, the subject is at risk of developing cancer. In some embodiments, the subject has cancer. In some embodiments, the subject has been diagnosed with cancer. In some embodiments, the subject is at risk of developing cancer. In some embodiments, the subject is at risk of developing cancer. In some embodiments, the subject has been treated for cancer. In some embodiments, the subject is being treated for cancer. In some embodiments, the subject is a cancer patient. In some embodiments, the subject is a cancer patient that is undergoing and/or being treated with chemotherapy.

In some embodiments, the subject is selected from the group consisting of a subject suspected of having cancer, a subject that has cancer, a subject diagnosed with cancer, a subject that is at risk of developing cancer, a subject that has been treated for cancer, and a subject that is being treated for cancer.

“Mammal,” as used herein, refers to any member of the class Mammalia, including, without limitation, humans and nonhuman primates such as chimpanzees and other apes and monkey species; farm animals such as cattle, sheep, pigs, goats and horses; domesticated mammals, such as dogs and cats; laboratory animals including rodents such as mice, rats and guinea pigs, and the like. The term does not denote a particular age or sex. Thus, adult and newborn subjects, whether male or female, are intended to be included within the scope of this term.

By “at risk of” is intended to mean at increased risk of, compared to a normal subject, or compared to a control group, e.g. a patient population, or a reference. Thus a subject carrying a particular marker may have an increased risk for a specific symptom, disease condition, disease, or disorder, and be identified as needing further testing. “Increased risk” or “elevated risk” mean any statistically significant increase in the probability, e.g., that the subject has the symptom, disease, disorder, or disease condition. In some embodiments, the risk is increased by at least 10% over the control group or reference with which the comparison is being made. In some embodiments, the risk is increased by at least 20% over the control group or reference with which the comparison is being made. In some embodiments, the risk is increased by at least 50% over the control group or reference with which the comparison is being made.

In some embodiments, the reference is selected from: (i) a control subject or a sample from the control subject, wherein the control subject does not have the disease, disorder, or disease condition; (ii) a control subject or a sample from the control subject, wherein the control subject has the disease, disorder, or disease condition; (iii) the subject or a sample from the subject that was obtained from the subject at an earlier point in time; (iv) a healthy subject or a sample from the healthy subject; an (v) the subject or a sample from the subject after the subject was treated for the disease, disorder, or disease condition.

The term “statistically significant” or “significantly” refers to statistical evidence that there is a difference. It is defined as the probability of making a decision to reject the null hypothesis when the null hypothesis is actually true. The decision is often made using the p-value.

“Antibody” refers to a polypeptide ligand substantially encoded by an immunoglobulin gene or immunoglobulin genes, or fragments thereof, which specifically binds and recognizes an epitope (e.g., an antigen). The recognized immunoglobulin genes include the kappa and lambda light chain constant region genes, the alpha, gamma, delta, epsilon and mu heavy chain constant region genes, and the myriad immunoglobulin variable region genes. Antibodies exist, e.g., as intact immunoglobulins or as a number of well characterized fragments produced by digestion with various peptidases. This includes, e.g., Fab′ and F(ab)′.sub.2 fragments. The term “antibody,” as used herein, also includes antibody fragments either produced by the modification of whole antibodies or those synthesized de novo using recombinant DNA methodologies. It also includes polyclonal antibodies, monoclonal antibodies, chimeric antibodies, humanized antibodies, or single chain antibodies. “Fc” portion of an antibody refers to that portion of an immunoglobulin heavy chain that comprises one or more heavy chain constant region domains, CH1, CH2 and CH3, but does not include the heavy chain variable region.

“Sample” is used herein in its broadest sense. The term “biological sample” as used herein denotes a sample taken or isolated from a biological organism. A sample or biological sample may comprise a bodily fluid including blood, serum, plasma, tears, aqueous and vitreous humor, spinal fluid; a soluble fraction of a cell or tissue preparation, or media in which cells were grown; or membrane isolated or extracted from a cell or tissue; polypeptides, or peptides in solution or bound to a substrate; a cell; a tissue; a tissue print; a fingerprint, skin or hair; fragments and derivatives thereof. Non-limiting examples of samples or biological samples include cheek swab; mucus; whole blood, blood, serum; plasma; urine; saliva; semen; lymph; fecal extract; sputum; other body fluid or biofluid; cell sample; and tissue sample etc. The term also includes a mixture of the above-mentioned samples or biological samples. The term “sample” also includes untreated or pretreated (or pre-processed) biological samples. In some embodiments, a sample or biological sample can comprise one or more cells from the subject. In some embodiments, a sample or biological sample can comprise one or more tissue samples from the subject. In some embodiments, a sample or biological sample is a tissue or tissue sample. In some embodiments, a sample or biological sample can be a tumor cell sample, e.g. the sample can comprise cancerous cells, cells from a tumor, and/or a tumor biopsy.

In some embodiments, a sample can comprise one or more cells from the subject. In some embodiments, the sample can comprise one or more tissues from the subject. In some embodiments, a sample is a cell or cell sample. In some embodiments, a sample is a tissue or tissue sample. In some embodiments, the sample is a tumor, tumor tissue, or tumor cell. In some embodiments, the sample is a cancer cell or cancer tissue. In some embodiments, a sample can be a tumor cell sample, e.g. the sample can comprise cancerous cells, cancer cells, cells from a tumor, and/or a tumor biopsy. In some embodiments, the tissue is a cancer tissue. In some embodiments, the tissue is a tumor tissue. In some embodiments, the cell is a cancer cell. In some embodiments, the cell is a tumor cell.

Non-limiting examples of samples or biological samples include, cheek swab; mucus; whole blood, blood, serum; plasma; blood products, urine; saliva; semen; lymph; fecal extract; sputum; other body fluid or biofluid; cell sample; tissue sample; tissue extract; tissue biopsy etc.

In some embodiments, samples or biological samples comprise blood products, including whole blood, blood, plasma and/or serum. In some embodiments, samples or biological samples comprise derivatives of blood products, including whole blood, blood, plasma and/or serum. In some embodiments, the sample is a biological sample. In some embodiments, the sample is whole blood. In some embodiments, the sample is blood. In some embodiments, the sample is plasma. In some embodiments, the sample is serum.

In some embodiments, the sample is a tissue sample. In some embodiments, the sample is a tissue extract. In some embodiments the sample is a biopsy sample. In some embodiments the sample is a biopsy specimen.

The terms “body fluid” or “bodily fluids” are liquids originating from inside the bodies of organisms. Bodily fluids include amniotic fluid, aqueous humour, vitreous humour, bile, whole blood, blood (e.g., serum, plasma), breast milk, cerebrospinal fluid, cerumen (earwax), chyle, chyme, endolymph and perilymph, exudates, feces, female ejaculate, gastric acid, gastric juice, lymph, mucus (e.g., nasal drainage and phlegm), pericardial fluid, peritoneal fluid, pleural fluid, pus, rheum, saliva, sebum (skin oil), serous fluid, semen, smegma, sputum, synovial fluid, sweat, tears, urine, vaginal secretion, and vomit. Extracellular bodily fluids include intravascular fluid (blood plasma), interstitial fluids, lymphatic fluid and transcellular fluid. Immunoglobulin G (IgG), the most abundant antibody subclass, may be found in all body fluids. “Biological sample” also includes a mixture of the above-mentioned body fluids. “Biological samples” may be untreated or pretreated (or pre-processed) biological samples.

Sample collection procedures and devices known in the art are suitable for use with various embodiment of the present invention. Examples of sample collection procedures and devices include but are not limited to: phlebotomy tubes (e.g., a vacutainer blood/specimen collection device for collection and/or storage of the blood/specimen), dried blood spots, Microvette CB300 Capillary Collection Device (Sarstedt), HemaXis blood collection devices (microfluidic technology, Hemaxis), Volumetric Absorptive Microsampling (such as CE-IVD Mitra microsampling device for accurate dried blood sampling (Neoteryx), HemaSpot™-HF Blood Collection Device. Additional sample collection procedures and devices include but are not limited to: a tissue sample collection device; standard collection/storage device (e.g., a collection/storage device for collection and/or storage of a sample (e.g., blood, plasma, serum, urine, etc.); a dried blood spot sampling device. In some embodiments, the Volumetric Absorptive Microsampling (VAMS™) samples can be stored and mailed, and an assay can be performed remotely.

As used herein, the term “amino acid” refers to naturally occurring and synthetic amino acids, as well as amino acid analogs and amino acid mimetics that operate in a manner similar to the naturally occurring amino acids. Naturally occurring amino acids are those encoded by the genetic code, as well as those amino acids that are later modified, e.g., hydroxyproline, -carboxyglutamate, and O-phosphoserine. Amino acid analogs refer to compounds that have the same basic chemical structure as a naturally occurring amino acid, i.e., an carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that operates in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

A protein refers to any of a class of nitrogenous organic compounds that comprise large molecules composed of one or more long chains of amino acids and are an essential part of all living organisms. A protein may contain various modifications to the amino acid structure such as disulfide bond formation, phosphorylations and glycosylations. A linear chain of amino acid residues may be called a “polypeptide.” A protein contains at least one polypeptide. Short polypeptides, are sometimes referred to as “peptides.”

The term “peptide” as used herein refers to a polymer of amino acid residues typically ranging in length from 2 to about 30, or to about 40, or to about 50, or to about 60, or to about 70 residues. In certain embodiments the peptide ranges in length from about 2, 3, 4, 5, 7, 9, 10, or 11 residues to about 60, 50, 45, 40, 45, 30, 25, 20, or 15 residues. In certain embodiments the peptide ranges in length from about 8, 9, 10, 11, or 12 residues to about 15, 20 or 25 residues. In certain embodiments the amino acid residues comprising the peptide are “L-form” amino acid residues, however, it is recognized that in various embodiments, “D” amino acids can be incorporated into the peptide. Peptides also include amino acid polymers in which one or more amino acid residues are an artificial chemical analogue of a corresponding naturally occurring amino acid, as well as to naturally occurring amino acid polymers. In addition, the term applies to amino acids joined by a peptide linkage or by other, “modified linkages” (e.g., where the peptide bond is replaced by an a-ester, a f3-ester, a thioamide, phosphonamide, carbamate, hydroxylate, and the like (see, e.g., Spatola, (1983) Chern. Biochem. Amino Acids and Proteins 7: 267-357), where the amide is replaced with a saturated amine (see, e.g., Skiles et al., U.S. Pat. No. 4,496,542, which is incorporated herein by reference, and Kaltenbronn etal., (1990) Pp. 969-970 in Proc. 11th American Peptide Symposium, ESCOM Science Publishers, The Netherlands, and the like)).

The term “threshold” as used herein refers to the magnitude or intensity that must be exceeded for a certain reaction, phenomenon, result, or condition to occur or be considered relevant. The relevance can depend on context, e.g., it may refer to a positive, reactive or statistically significant relevance.

The term “disease” refers to an abnormal condition affecting the body of an organism. For example, the disease or abnormal condition may result from a pathophysiological response to external or internal factors.

The term “disorder” refers to a functional abnormality or disturbance. For example, a disorder may be a disruption of the disease to the normal or regular functions in the body or a part of the body.

The term “disease condition” refers to an abnormal state of health that interferes with the usual activities of feeling or wellbeing

The term “normal condition” or “healthy condition” refers to a normal state of health.

The term “healthy state” or “normal state” means that the state of the subject (e.g., biological state or health state, etc.) is not abnormal or does not comprise a disease, disorder, or disease condition.

A “healthy subject” or “normal subject” is a subject that does not have a disease, disorder, or disease condition.

The term “unhealthy subject” or “abnormal subject” is a subject that does have a disease, disorder, or disease condition.

“Diseases”, “disorders” and “disease conditions,” as used herein may include, but are in no way limited to any form of a cancer.

In various embodiments, the disease is at least one cancer. In various embodiments, the disorder is at least one cancer. In various embodiments, the disease condition is at least one cancer.

Examples of cancer include but are not limited to breast cancer such as a ductal carcinoma in duct tissue in a mammary gland, medullary carcinomas, colloid carcinomas, tubular carcinomas, and inflammatory breast cancer; ovarian cancer, including epithelial ovarian tumors such as adenocarcinoma in the ovary and an adenocarcinoma that has migrated from the ovary into the abdominal cavity; cervical cancers such as adenocarcinoma in the cervix epithelial including squamous cell carcinoma and adenocarcinomas; prostate cancer, such as a prostate cancer selected from the following: an adenocarcinoma or an adenocarinoma that has migrated to the bone; pancreatic cancer such as epitheliod carcinoma in the pancreatic duct tissue and an adenocarcinoma in a pancreatic duct; bladder cancer such as a transitional cell carcinoma in urinary bladder, urothelial carcinomas (transitional cell carcinomas), tumors in the urothelial cells that line the bladder, squamous cell carcinomas, adenocarcinomas, and small cell cancers; acute myeloid leukemia (AML), preferably acute promyleocytic leukemia in peripheral blood; lung cancer such as non-small cell lung cancer (NSCLC), which is divided into squamous cell carcinomas, adenocarcinomas, and large cell undifferentiated carcinomas, and small cell lung cancer; skin cancer such as basal cell carcinoma, melanoma, squamous cell carcinoma and actinic keratosis, which is a skin condition that sometimes develops into squamous cell carcinoma; eye retinoblastoma; intraocular (eye) melanoma; primary liver cancer (cancer that begins in the liver); kidney cancer; thyroid cancer such as papillary, follicular, medullary and anaplastic; AIDS-related lymphoma such as diffuse large B-cell lymphoma, B-cell immunoblastic lymphoma and small non-cleaved cell lymphoma; Kaposi's sarcoma; Ewing sarcoma; central nervous system cancers such as primary brain tumor, which includes gliomas (astrocytoma, anaplastic astrocytoma, or glioblastoma multiforme (GBM)), Oligodendroglioma, Ependymoma, Meningioma, Lymphoma, Schwannoma, and Medulloblastoma; peripheral nervous system (PNS) cancers such as acoustic neuromas and malignant peripheral nerve sheath tumor (MPNST) including neurofibromas and schwannomas; oral cavity and oropharyngeal cancer; stomach cancer such as lymphomas, gastric stromal tumors, and carcinoid tumors; testicular cancer such as germ cell tumors (GCTs), which include seminomas and nonseminomas; and gonadal stromal tumors, which include Leydig cell tumors and Sertoli cell tumors; head cancer; neck cancer; throat cancer; and thymus cancer, such as to thymomas, thymic carcinomas, Hodgkin disease, non-Hodgkin lymphomas carcinoids or carcinoid tumors. Also, the methods can be used to treat viral-induced cancers. The major virus-malignancy systems include hepatitis B virus (HBV), hepatitis C virus (HCV), and hepatocellular carcinoma; human lymphotropic virus-type 1 (HTLV-1) and adult T-cell leukemia/lymphoma; and human papilloma virus (HPV) and cervical cancer. In some embodiments, the cancer is metastasized. In some embodiments, the cancer is glioma. In some embodiments, the glioma is selected from the group consisting of astrocytoma, anaplastic astrocytoma, glioblastoma multiforme (GBM), oligodendroglioma and combinations thereof.

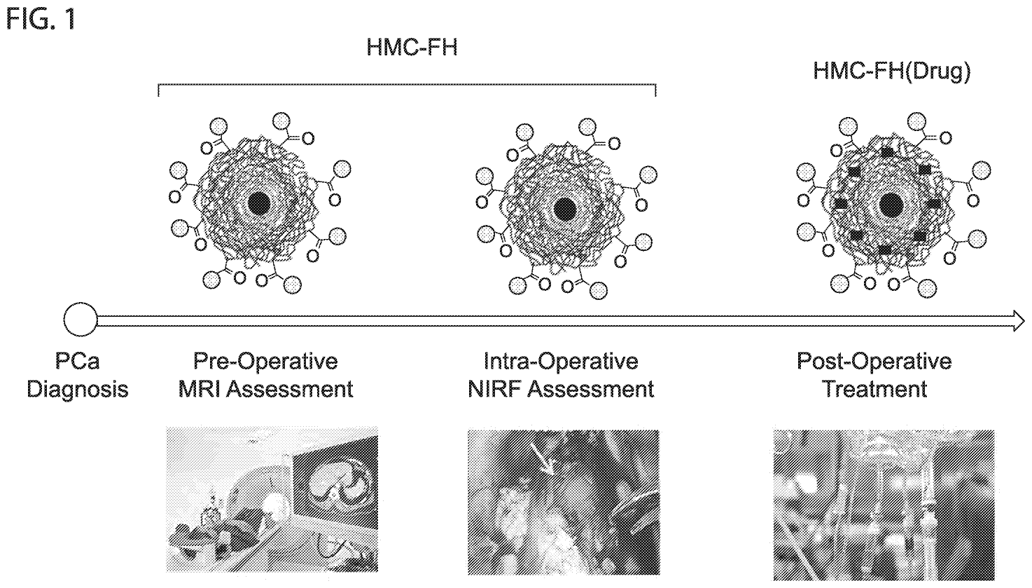

In various embodiments, the present invention relates to the development of an iron oxide nanoparticle based platform technology that would allow for (1) an MRI-based pre-surgery assessment of a tumor location and margins, (2) a fluorescent image-guided visualization of the tumor during surgery, and (3) and effective post-surgery chemotherapy regime to treat remaining primary tumor as well as metastatic lesions (FIG. 1). MRI is among the best pre-operative imaging technologies for PCa due to its high spatial and contrast resolution and the lack of ionizing radiation.^{[1]} It is typically used to determine the extent of the disease via the acquisition of a combination of T2-weighted and diffusion-weighted images. In addition, dynamic contrast-enhanced MRI using iron oxide nanoparticle formulations such as Feraheme (FH) would results in enhancement in tumor contrast and better detection on tumor margins and degree of tumor vascularization. Meanwhile, fluorescence imaging is the most promising approach for the intraoperative resection of tumors and sentinel lymph node metastasis.^{[2-12]} Intraoperative fluorescence-imaging provide guidance during cancer surgery for the complete resection of tumors with high sensitivity by identifying tumor margins during surgery. It is imperative that most if not all of the cancer tissue is taken out. For this to be accomplished, highly fluorescent agents that localize specifically to cancer are needed. However, even after successful resection of cancerous tissues, there is always the possibility that tissue, not-identified as cancerous during surgery, remains or cancer cells have already migrated through the lymphatic system to other organs to establish metastasis. Therefore, post-surgical chemotherapy typically is administered, resulting in an improved outcome and survival, minimizing recurrences and the establishment of metastasis.^{[13]} It would be highly advantageous, to utilize a nanoparticle based system that can aid in the visualization of tumors both pre-surgery and during surgery, while using the same nanoparticle platform technology as delivery system to deliver drug post-operatively. In various embodiments of the present invention, we disclose the use of a Feraheme (FH) based image-guided system for both the pre-operative and intra-operative assessment of PCa tumor margins as well as the post-surgical treatment and assessment of drug delivery to primary and secondary (metastasis) tumors using the same FH-based agent. To prove the use of our technology in cancer, we have done initial studies using cell cultures and mouse models of prostate and brain (glioblastoma) tumors. However, the technology can be used for the imaging and treatment of other solid tumors such as those from lungs, breast, ovaries, pancreas, head and neck, and skin among others.

In various embodiments, the present invention is based on the use of Feraheme (FH), an FDA-approved iron oxide nanoparticle formulation Feraheme (FH), also known as Ferumoxytol, is currently used in the clinic to treat iron deficiency anemia.^{[14]} FH is typically administered in two doses of 510 mg of iron each, between 3-8 days, for a total dose of 1020 mg Fe per treatment. The pharmacokinetics, biodistribution and safety profile of FH has been extensively studied, showing minimal toxicity in animal and humans subjects, being metabolized as regular iron by the liver within 6-8 weeks.^{[15, 16]} In addition, FH is increasingly used off-labeled in MR angiography and liver imaging due to its superparamagnetic properties, at doses far below those used for anemia treatment.^{[17-19]} Toxicity studies have shown that even a 12-fold higher than the clinical dose of FH present no significant toxicity with very few side effects being reported in adult cases.^{[16, 20]} Among those, anaphylaxis and hypersensitivity reactions are the most serious ones, but these problems have been minimized by administering FH as a diluted IV infusion over a period of 15 minutes or more as opposed to an undiluted bolus administration as it was administered in the past. In general, the use of FH is safe.^{[16]} In addition, iron oxide nanoparticles have been widely studied as magnetic sensors and most recently as drug delivery agents. Polymer coated iron oxide nanoparticles can encapsulate a hydrophobic cargo such as drugs (Taxol, Doxorubicin) or fluorescence dyes (DiI, DiR) within the nanoparticle's polymer coating (dextran or polyacrylic acid).^{[21]} The stable encapsulation of these cargos occurs at physiological pH within hydrophobic pockets in the nanoparticle's polymeric coating via hydrophobic and electrostatic interactions. At pH 6.5 or below, release of the cargo occurs, either fluorescently labeling the cell or causing cell death, when either a fluorescent dye or a cytotoxic drug was encapsulated respectively. Feraheme (FH) itself can be used as a drug delivery vehicle and that its superparamagnetic properties allow for MR-guided assessment of nanoparticle accumulation and drug release.^{[22]} In addition, our data shows that a FH-encapsulated drug is more efficient in reducing the size of tumors than the drug alone. These results were similar with all encapsulated drug such as doxorubicin, paclitaxel and bortezomib.

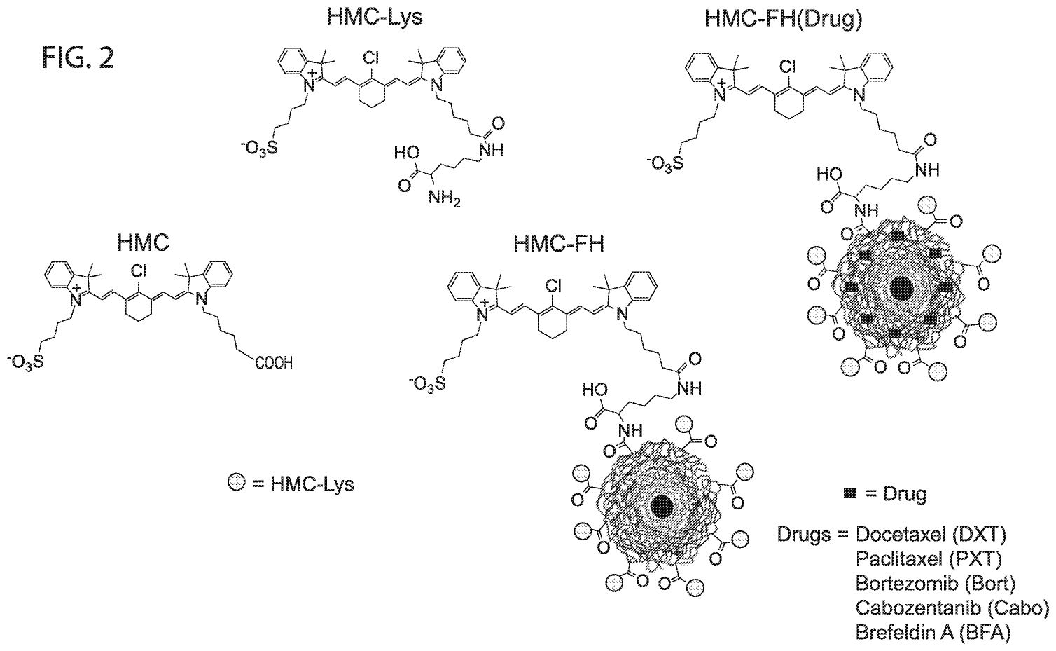

Even though tumor accumulation of nanoparticle via Enhanced Permeability and Retention (EPR) effect is widely recognized to be effective for nanoparticle-drug delivery, it is not universal for all tumors. Furthermore, crossing the brain blood/tumor barrier is a challenge to overcome when treating brain tumors such as glioblastomas. Tumor targeting and enhanced brain blood barrier transcytosis can occur via receptor mediated targeting, which is facilitated by the attachment of targeting ligands to the nanoparticle surface.^{[23, 24]} In FH, carboxylic acid groups on the nanoparticle surface can be further modified with targeting ligands for specific targeting and accumulation in tumors. Of a wide selection of ligands that one can choose to target tumors, we selected the heptamethine carbocyanine (HMC) ligand to conjugate to FH (FIG. 2). HMC targets the organic anion transporter peptides (OATPs) which are a superfamily of transmembrane glycoproteins overexpressed in various tumors.^{[25, 26]} The OATPs family of proteins is composed of various subtypes including 11 known human OATPs classified into 6 subfamilies based on their amino acid sequence homologies.^{[25, 27]} For example, the OATP1B3 and OATP1A2 subtypes have been shown to be overexpressed in prostate cancer,^{[28, 29]} while OATP1A2 and OATP2B1 have been found to be expressed in brain tumors and brain metastasis.^{[25, 27, 28]} OATPs facilitate the transport of several substances into cells, including drugs and hormones.^{[25, 27]} Although the actual mechanism of HMC uptake by multiple tumors has not been fully elucidated, it is believed that the selective overexpression of multiple subtypes of OATPs in tumors contribute to the HMC ligand uptake by tumors. For example, it has been demonstrated that the overexpression of OATP1B3 mediate the selective uptake of HMC ligands in prostate cancer cells, but not in normal prostate epithelial cells.^{[30]} Therefore, the OATP1B3 subtype may be the transporter predominantly involved in the selective uptake of HMC in prostate cancer. HMC is a unique ligand because it also exhibits near infrared fluorescence (NIRF), with excitation in 750 nm and emission in 800. The dual NIRF imaging and OATP-targeting capability of HMC is unique and upon conjugation to Feraheme will endow FH with dual NIRF- and MR-imaging capabilities, as well as OATP-targeting ability. In addition to HMC unique NIRF properties, it has been shown that this ligand preferentially accumulates in a variety of cancer cells, but not normal cells as demonstrated in a variety of cancer cell lines, tumor xenografts, spontaneous mouse tumors in transgenic animals and human tumor samples.^{[31-33]} The HMC uptake has also been found to be mediated by tumor hypoxia and activated (HIF1α)/OATP signaling.^{[34]} Given that hypoxia and aberrant expression of OATPs is shared by multiple types of tumors and their metastatic lesion, conjugation of HMC on the surface on Feraheme will facilitate the pre-operative detection of tumors by MRI and the intraoperative detection of tumor margins by fluorescence imaging, while allowing for the post-surgical delivery of drugs to primary and secondary (metastasis) tumors.

Prostate Cancer (PCa). Challenges in PCa treatment. PCa remains one of the leading causes of death in men in the USA and around the world.^{[35, 36]} Current cancer chemotherapeutics, along with antiandrogen therapy, have improved the long-term survival of these patients.^{[13]} However, surgical removal of the cancerous tissue continues to be the most effective approach, resulting in curative results when complete removal of the cancerous tissue is achieved and no metastasis to nearby lymph nodes and other organs have occurred. Currently, complete removal of the cancer tissue in prostate cancer is challenging due to the location and proximity of the prostate gland to other organs such as the bladder, rectum, urethra and prostatic nerves. These issues limit the adaptation of wide surgical margins during prostatectomy, often resulting in positive surgical margins in up to 48% of the cases that require the use of post-surgery adjuvant chemotherapy using Docetaxel (DXT) and prednisone.^{[37, 38]}

Glioblastoma Multiforme (GBM). Challenges in glioblastoma treatment. Despite advances in surgical resection, chemotherapy and radiation treatment of glioblastoma multiforme (GBM), the overall median survival is estimated to be only about 15 months with a five-year survival rate of 10% after radiation therapy and chemotherapy^{[39-41]}. GBM can affect both men and women equally and at any age. This statistic makes GBM one of the most lethal and aggressive cancers. Standard of care starts with surgery, to eliminate most of the tumor mass, followed by a combination of chemo and radiation therapy to eradicate any residual tumor tissue. Alkylating agents such as temozolomide, in combination with surgical tumor resection and radiotherapy have increased the overall survival of newly diagnosed patients, but only by expanding survival by a couple of months. Unfortunately, tumor recurrence often develops within a few months after treatment due to difficulties in establishing tumor margins during surgery and in inefficient post-surgical treatments using chemotherapy. The failure of most chemotherapies to treat GBM is due to the ineffective ability of most drugs to cross the brain blood barrier (BBB) within the tumor area, more specifically the brain tumor area. Most problematic, recurrent tumors after failed chemotherapy are typically resistant to both classical chemotherapy and radiation therapy^{[42-45]}, which makes treatment even more difficult. For these reasons, developing a nanoparticle based therapeutics that can (1) facilitate the visualization of tumors by MRI and fluorescent imaging pre and during surgery respectively, while (2) delivering potent chemotherapeutic drugs to the brain tumor are desperately needed. Overall, taxanes such as docetaxel and paclitaxel have been beneficial in the treatment most tumors, except for brain tumors due to the inability of these drugs to cross the brain blood barrier. Even though a taxane nanoformulation (Abraxane®) to treat other tumors via the EPR has been used to successfully treat other tumors, this formulation does not cross the BBB and it is not effective in treating GBM. For this reason, a nanoformulation that can deliver a taxane (DXT, PXL) to GBM cells by crossing the BBB would be a most needed improvement in the treatment of GBM. In this invention we report the use of HMC-FH(Drug) to deliver taxanes to GBM. Other drugs that typically do not cross the BBB such as Cabozentanib, Brefeldin A, and Bortexomib, among others could be delivered to brain tumors using the same platform technology.

In some embodiments, the drug is not a boron cluster. In some embodiments, the drug is not a compound comprising boron. In some embodiments, the drug does not comprise a boron cluster. In some embodiments, the drug does not comprise a compound comprising boron. In some embodiments, the drug does not contain a boron cluster. In some embodiments, the drug does not contain a compound comprising boron. In some embodiments, the drug does not comprise boron. In some embodiments, the drug does not contain boron.

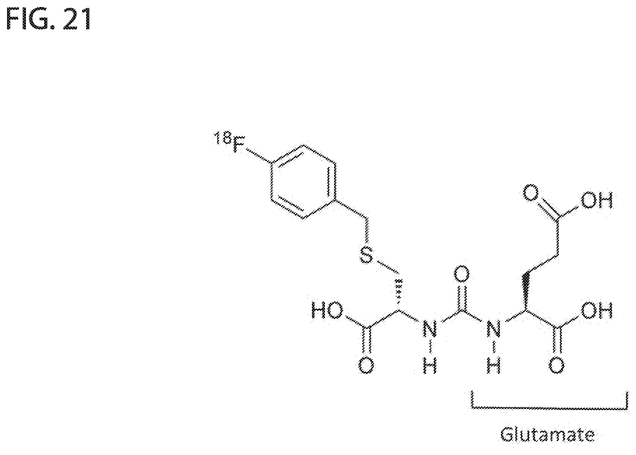

In various embodiments, the present invention relates to the use of conjugates of iron oxide nanoparticles with folic acid or glutamic acid for the multimodal detection of prostate cancer via direct targeting of the prostate specific membrane antigen (PSMA), which is overexpressed in both primary and metastatic prostate cancer as well as the neovasculature of most solid tumors, including breast, and lung, among others. PSMA has gained increasing interest as a molecular target for imaging as well as for the delivery of targeted cancer therapeutics. PSMA is a cell surface protein known to have a dual enzymatic activity of folate hydrolysate and glutamate carboxylase. PSMA binds folic acid, glutamic acid, and polyglutamated folates and facilitates the internalization of these molecules into cancer cells. Glutamic acid (glutamate) based molecule have been more extensively used to target PSMA than folic acid (folate) molecules. Indeed, various glutamate urea based probes have been designed to deliver optical and PET imaging agent (18F and 68Ga) to PCa tumors via PSMA. FIG. 21 shows the structure of one of these PSMA targeting imaging agents, 18F-DCFBC, where the glutamate moiety facilitates binding to PSMA.

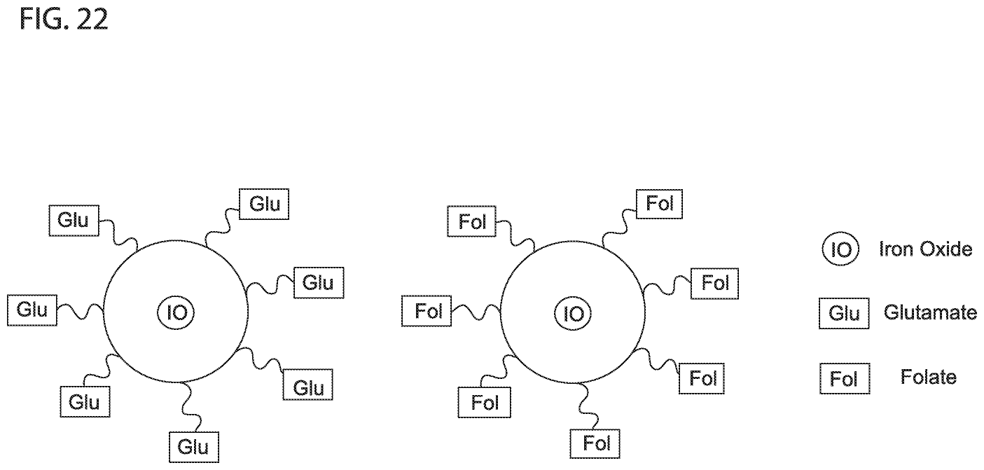

In this invention, glutamate (or folate) is covalently bound to iron oxide nanoparticle (Feraheme) to image PSMA in prostate cancer tumors (FIG. 22). A commercial and FDA-approved formulation of carboxymethyl dextran iron oxide nanoparticles, Feraheme (Ferumoxytol), was used in our invention. It is understood, however, that other versions of iron oxide nanoparticles can be also used besides Feraheme. Feraheme is used in the clinic to treat iron deficiency (anemia), but it is increasingly being used in MR-angiography and liver imaging.

In various embodiments of the present invention, the carboxylic acid groups on the surface of the Feraheme nanoparticles were conjugated to the amino group in glutamate to yield the Glu-Feraheme (GLU-FH) NP using EDC/NHS chemistry. Meanwhile, as folate does not have a functional amino group to conjugate directly to Feraheme, Folate-PEG-amine is used instead to yield Folate-PEG-Feraheme.

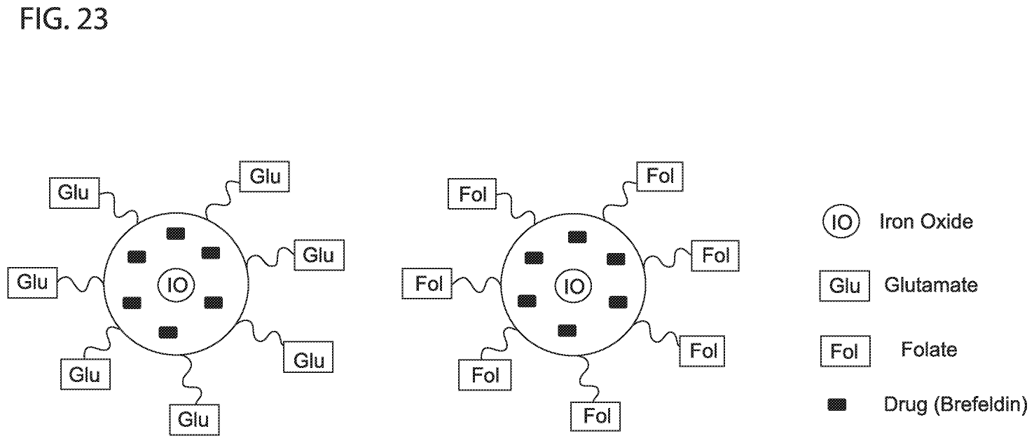

In another embodiment of the present invention, a theranostic nanoparticle has been developed (FIG. 23) by encapsulating a drug such as Brefeldin A within the carboxymethyl dextran coating of the PSMA targeting-Feraheme NPs. Folate ligands were attached to target the folate receptor. In various embodiments of the present invention, in addition to folic acid, glutamic acid is used to target the Feraheme nanoparticles to prostate cancer via PSMA. Therefore, Glutamate-Feraheme and Folate-Feraheme (Fol-FH) were synthesized and tested to target prostate cancer via PSMA for imaging and/or as a therapeutic to deliver BFA to prostate cancer. In addition, polyacrylic acid coated iron oxide nanoparticle can encapsulate or entrap drugs within the polymeric coating, creating a multimodal and theranostic nanoparticle.

In various embodiments of the present invention we report the use of GLU-FH or FOL-FH to encapsulate Brefeldin-A. Brefeldin, a promising drug patented by the NCI in 1997 (U.S. Pat. No. 5,696,154), has been extensively studied as an anticancer drug. Brefeldin inhibits protein trafficking and transport from the endoplasmic reticulum to the Golgi apparatus, causing activation of the unfolded protein response (UPR) and endoplasmic reticulum stress (ER-stress), which result in cell death by apoptosis. The known biological target of Brefeldin within the ER is ADP ribosylation factor 1 (ARF-1), a member of the RAS family of proteins that regulates the formation of protein transport vesicles within the ER. ARF-1 has been found to be elevated in various tumors and associated with invasion and metastasis. Therefore, ARF-1 in a good target for cancer therapy. A crystal structure of ARF-1 binding Brefeldin A has been reported. Brefeldin A has been shown to induce cell death by apoptosis or cell arrest in various cancer cell lines of leukemia, breast, colon, prostate, lung and brain, among others. In particular, it has been shown to inhibit the growth and migration of cancer stem cell. Unfortunately, the hydrophobic (water-insoluble) nature of this drugs hampers its successful intravenous administration to maintain therapeutic plasma concentrations that effectively kill tumors with minimal side effects. Therefore, novel ways to administer and target Brefeldin A to tumors are needed.

In various embodiments, the present invention relates to the use of conjugates of iron oxide nanoparticles with at least one Angiopep. An Angiopep is a peptide that has been described in the literature to cross the brain blood barrier (BBB). Non-limiting examples of Angiopeps include Angiopep-1, Angiopep-2, Angiopep-5, or Angiopep-7. In some embodiments, at least one Angiopep is selected from Angiopep-1, Angiopep-2, Angiopep-5, Angiopep-7, and combinations thereof. Angiopep-2 is a 19 amino acid peptide (TFFYGGSRGKRNNFKTEEY) (SEQ ID NO: 2) that binds to the low-density lipoprotein receptor-related protein 1 (LRP-1), which is highly expressed in the brain endothelial cells of the BBB. Upon binding of Angiopep to LRP-1, the whole complex crosses the BBB via a transcytosis mechanism. Transcytosis typically enables the transport of proteins through the BBB via the formation of membrane-bound vesicles. In the case of LRP-1, these vesicles form upon binding of lipoproteins to this receptor on the apical side of the endothelia and quickly move to the basolateral side where the vesicles fuse with the membrane, releasing the cargo within the brain. Furthermore, glioblastoma multiforme (GBM) and other forms of malignant brain tumors have been found to have increased expression of LRP-1. In the particular case of GBM, studies have found that LRP-1 induces the expression of matrix metalloproteinase 2 (MMP2) and MMP9, promoting migration and invasion of human GBM cells (U87). Therefore, LRP-1 is an excellent target to facilitate the crossing of nanotherapeutics through the BBB, as well as their binding and internalization within brain cancer cells. Angiopep has been found to bind to LRP-1 and transcytose across the BBB.

Angiopep-1 is a peptide with the following amino acid sequence:

(SEQ ID NO: 3)
TFFYGGCRGKRNNFKTEEY.

Angiopep-2 is a peptide with the following amino acid sequence:

(SEQ ID NO: 2)
TFFYGGSRGKRNNFKTEEY.

Angiopep-5 is a peptide with the following amino acid sequence:

(SEQ ID NO: 4)
TFFYGGSRGKRNNFRTEEY.

Angiopep-7 is a peptide with the following amino acid sequence:

(SEQ ID NO: 5)
TFFYGGSRGRRNNFRTEEY.

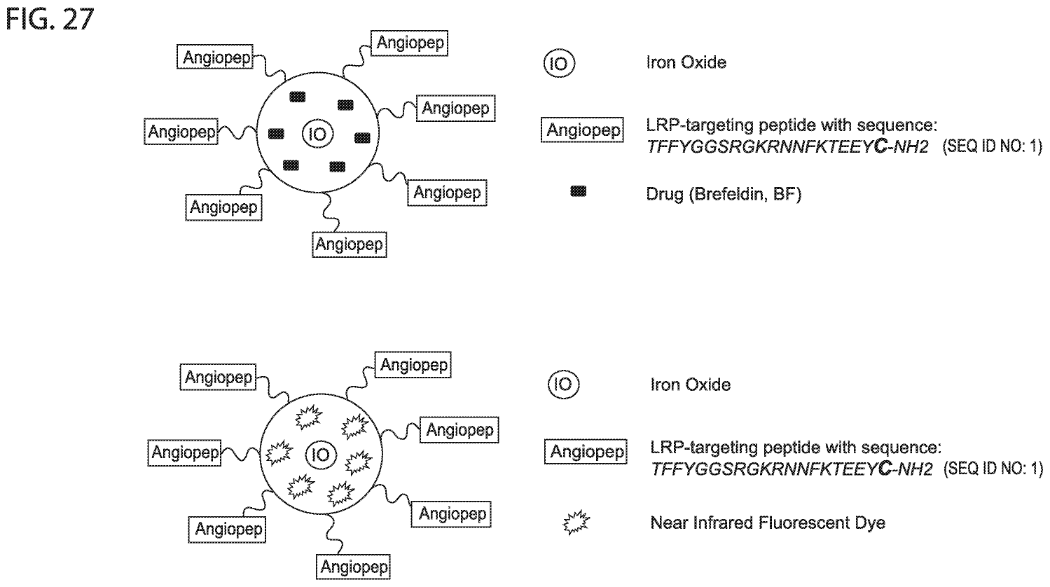

In various embodiments of the present invention, Feraheme (Ferumoxytol) a commercial and FDA-approved formulation of carboxymethyl dextran iron oxide nanoparticles, was conjugated with Angiopep-2 and encapsulated with either a near infrared dye (DiI or DiR) or a drug (Brefeldin or Paclitaxel) for the delivery of this cargo through the BBB (FIG. 27). By conjugating Angiopep-2 to Feraheme, an Angiopep-Feraheme nanoparticle conjugate will be produced with the following properties: 1. LRP-1 mediated transcytosis of Feraheme across the BBB; and 2. The use of Angiopep-Feraheme to deliver a cargo across the BBB. Brefeldin A is used herein as a model drug, but other drugs such as paclitaxel, vincristine, or temozolomide, among others, can be encapsulated.

In various embodiments of the present invention, we have conjugated Angiopep to the surface of Feraheme. Angiopep is a peptide that target the LRP-1 receptors which is overexpressed on the brain blood barrier (BBB) and on the cells of most brain tumors. The resulting Angiopep-Feraheme nanoparticle can then encapsulate drugs (such as brefeldin-A) or fluorescent dyes (e.g., DiI or DiR), among other cargos, for their delivery across the BBB and into brain tumor cells. In various embodiments of the present invention, we have data that show that Angiopep facilitates the delivery of a fluorescent dye and a drug (brefeldin) into human brain vascular endothelial cells (HBMVEC), glioblastoma multiforme (GBM) cell lines. The Angiopep-Feraheme (BFA)-formulation affect the U87 cancer cells lines as well as a GBM stem cell line in the nanomolar range. In various embodiments, of the present invention delivery of other drugs to LRP-1 expressing brain tumors may also be used. In various embodiments of the present invention, delivery or drug delivery to the brain can be monitored by MRI, as the magnetic properties of Feraheme allows for the monitoring of nanoparticle localization via MRI. In some embodiments, Angiopep is selected from the group consisting of Angiopep-1, Angiopep-2, Angiopep-5, and Angiopep-7, and combinations thereof. In some embodiments, Angiopep is Angiopep-2.

Various Non-Limiting Embodiments of the Invention

Nanoparticles, Compositions, and Articles of Manufacture

In various embodiments, the present invention provides a nanoparticle, comprising: a core, wherein the core comprises at least one iron oxide; a shell surrounding the core, wherein the shell comprises at least one polymer; and at least one targeting moiety attached to the shell.

In various embodiments, the present invention provides a nanoparticle, comprising: a core, wherein the core comprises at least one iron oxide; a shell surrounding the core, wherein the shell comprises at least one polymer. In some embodiments, the nanoparticle further comprises at least one targeting moiety. In some embodiments, the targeting moiety is attached to the shell.

In some embodiments, the nanoparticle does not comprise a boron cluster. In some embodiments, the nanoparticle does not contain a boron cluster. In some embodiments, a boron cluster is not encapsulated in the at least one polymer. In some embodiments, a boron cluster is not linked to the at least one polymer. In some embodiments, the nanoparticle does not contain boron. In some embodiments, the nanoparticle does not comprise boron.

In various embodiments, the present invention provides a composition, comprising: a core, wherein the core comprises at least one iron oxide; a shell surrounding the core, wherein the shell comprises at least one polymer; and at least one targeting moiety attached to the shell. In some embodiments, the composition is a nanoparticle.

In various embodiments, the present invention provides a composition, comprising: a core, wherein the core comprises at least one iron oxide; a shell surrounding the core, wherein the shell comprises at least one polymer. In some embodiments, the composition further comprises at least one targeting moiety. In some embodiments, the targeting moiety is attached to the shell.

In some embodiments, the composition does not comprise a boron cluster. In some embodiments, the composition does not contain a boron cluster. In some embodiments, a boron cluster is not encapsulated in the at least one polymer. In some embodiments, a boron cluster is not linked to the at least one polymer. In some embodiments, the composition does not contain boron. In some embodiments, the composition does not comprise boron.

In various embodiments, the present invention provides an article of manufacture, comprising: a core, wherein the core comprises at least one iron oxide; a shell surrounding the core, wherein the shell comprises at least one polymer; and at least one targeting moiety attached to the shell. In some embodiments, the article of manufacture is a nanoparticle.

In various embodiments, the present invention provides an article of manufacture, comprising: a core, wherein the core comprises at least one iron oxide; a shell surrounding the core, wherein the shell comprises at least one polymer. In some embodiments, the article of manufacture further comprises at least one targeting moiety. In some embodiments, the targeting moiety is attached to the shell.

In some embodiments, the article of manufacture does not comprise boron cluster. In some embodiments, the article of manufacture does not contain a boron cluster. In some embodiments, a boron cluster is not encapsulated in the at least one polymer. In some embodiments, a boron cluster is not linked to the at least one polymer. In some embodiments, the article of manufacture does not comprise boron. In some embodiments, the article of manufacture does not contain boron.

In various embodiments, the present invention provides a nanoparticle, comprising: ferumoxytol; and at least one targeting moiety. In some embodiments, the ferumoxytol comprises carboxymethyl dextran. In some embodiments, the nanoparticle does not comprise a boron cluster. In some embodiments, the nanoparticle does not contain a boron cluster. In some embodiments, a boron cluster is not encapsulated in the carboxymethyl dextran. In some embodiments, a boron cluster is not linked to the carboxymethyl dextran. In some embodiments, a boron cluster is not encapsulated in the ferumoxytol. In some embodiments, a boron cluster is not linked to the ferumoxytol.

In various embodiments, the present invention provides a nanoparticle, comprising: ferumoxytol. In some embodiments, the nanoparticle further comprises at least one targeting moiety.

In various embodiments, the present invention provides a nanoparticle, comprising at least one selected from the group consisting of Ferumoxytol, Ferumoxides, Ferucarbotran, Ferumoxtran-10, NC100150, VSOP C184, and combinations thereof; and at least one targeting moiety. In various embodiments, the present invention provides a composition, comprising at least one selected from the group consisting of Ferumoxytol, Ferumoxides, Ferucarbotran, Ferumoxtran-10, NC100150, VSOP C184, and combinations thereof; and at least one targeting moiety. In various embodiments, the present invention provides an article of manufacture, comprising at least one selected from the group consisting of Ferumoxytol, Ferumoxides, Ferucarbotran, Ferumoxtran-10, NC100150, VSOP C184, and combinations thereof; and at least one targeting moiety.

In various embodiments, the present invention provides a nanoparticle, composition, or article of manufacture comprising at least one selected from the group consisting of Ferumoxytol, Ferumoxides, Ferucarbotran, Ferumoxtran-10, NC100150, VSOP C184, and combinations thereof. In some embodiments, the nanoparticle, composition, or article of manufacture further comprises at least one targeting moiety.

In various embodiments, the present invention provides a nanoparticle, comprising: a core; a coating surrounding the core; and at least one targeting moiety. In various embodiments, the present invention provides a composition, comprising: a core; a coating surrounding the core; and at least one targeting moiety. In various embodiments, the present invention provides an article of manufacture, comprising: a core; a coating surrounding the core; and at least one targeting moiety.

In various embodiments, the present invention provides a nanoparticle, composition, or article of manufacture comprising: a core; and a coating surrounding the core. In some embodiments, the nanoparticle, composition, or article of manufacture further comprises at least one targeting moiety.

In various embodiments, the present invention provides a nanoparticle, comprising coated iron oxide or a coated iron oxide particle; and at least one targeting moiety.

In various embodiments, the present invention provides a nanoparticle, composition, or article of manufacture, comprising coated iron oxide or a coated iron oxide particle. In some embodiments, the nanoparticle, composition, or article of manufacture further comprises at least one targeting moiety.

In some embodiments, the coated iron oxide particle is selected from the group consisting of Ferumoxytol, Ferumoxides, Ferucarbotran, Ferumoxtran-10, NC100150, VSOP C184, and combinations thereof. In some embodiments, the coated iron oxide is selected from the group consisting of Ferumoxytol, Ferumoxides, Ferucarbotran, Ferumoxtran-10, NC100150, VSOP C184, and combinations thereof. In some embodiments, the coating comprises at least one selected from the group consisting of polymer, co-polymer, monomer, and combinations thereof.

In some embodiments, the shell comprises at least one selected from the group consisting of polymer, co-polymer, monomer, and combinations thereof.

In some embodiments, the core comprises at least one iron oxide.

In some embodiments, the nanoparticle optionally further comprises at least one drug.

In some embodiments, the nanoparticle optionally further comprises at least one fluorescent dye.

In some embodiments, the nanoparticle is a multimodal probe. In some embodiments, the nanoparticle is a multimodal nanoparticle. In some embodiments, the nanoparticle may be used for multimodal detection of a cancer in a subject. In some embodiments, the nanoparticle may be used for multimodal detection of a tumor in a subject. In some embodiments, the nanoparticle may be used for multimodal detection of a tumor margin of a tumor in a subject. In some embodiments, the nanoparticle may be used to deliver a drug for example to a cancer cell, cancer tissue, cancerous cell, cancerous tissue, or tumor.

In some embodiments, the nanoparticles of the present invention may be used to determine tumor concentration in a subject. In some embodiments, the nanoparticles of the present invention may be for dual visualization by magnetic resonance imaging (MRI) and fluorescence imaging. In some embodiments, the nanoparticles of the present invention may be used as markers during fluorescence image guided surgery for the intraoperative detection of tumor margins. In some embodiments, the nanoparticles of the present invention may be used to visualize drug delivery by magnetic resonance imaging and/or fluorescence imaging. In some embodiments, the fluorescence imaging is selected from the group consisting of near infrared fluorescence imaging, intraoperative fluorescence imaging, and combinations thereof.

Nanoparticles of the present invention may be administered to a subject (and thereby contacted with a tissue), or contacted with a tissue in vivo or in vitro. Thus, in some embodiments, the methods are applicable to both human therapy and veterinary applications, as well as research applications in vitro or within animal models.

In some embodiments, the nanoparticles of the present invention do not comprise a boron cluster. In some embodiments, the nanoparticles of the present invention do not contain a boron cluster.

In some embodiments, the nanoparticles of the present invention do not comprise a compound comprising boron. In some embodiments, the nanoparticles of the present invention do not contain a compound comprising boron.

In some embodiments, the compositions of the present invention do not comprise a boron cluster. In some embodiments, the compositions of the present invention do not contain a boron cluster.

In some embodiments, the compositions of the present invention do not comprise a compound comprising boron. In some embodiments, the compositions of the present invention do not contain a compound comprising boron.

In some embodiments, the articles of manufacture of the present invention do not comprise a boron cluster. In some embodiments, the articles of manufacture of the present invention do not contain a boron cluster.

In some embodiments, the articles of manufacture of the present invention do not comprise a compound comprising boron. In some embodiments, the articles of manufacture of the present invention do not contain a compound comprising boron.

In some embodiments, the nanoparticles, compositions, and/or articles of manufacture of the present invention selectively target and/or bind to diseased tissue and/or diseased cells. In some embodiments, the nanoparticles, compositions, and/or articles of manufacture of the present invention selectively target and/or bind to cancerous tissue, cancer tissue, cancer cells, tumor, tumor tissue, tumor cells, and combinations thereof.

In some embodiments, the nanoparticles, compositions, and/or articles of manufacture of the present invention selectively targets and/or binds to diseased tissue and/or diseased cells compared to non-diseased tissue and/or non-diseased cells. In some embodiments, the nanoparticles, compositions, and/or articles of manufacture of the present invention selectively targets and/or binds to cancerous tissue, cancer tissue, cancer cells, tumor, tumor tissue, tumor cells, and combinations thereof compared to healthy tissue, non-cancerous tissue, non-cancerous cells.

Iron Oxide Particles

Feraheme (FH), also known as Ferumoxytol, is an FDA-approved carboxymethyl dextran coated iron oxide nanoparticle formulation for the treatment of anemia. Feraheme (FH) is also used off-label as an MRI contrast agent. In various embodiments, Feraheme (FH) can be modified with targeting moieties to facilitate receptor mediated tumor accumulation or permeability through the brain blood barrier.

Non-limiting examples of coated iron oxide and/or coated iron oxide particles include Ferumoxytol (Feraheme®), Ferumoxides (Feridex® IV, Berlex Laboratories), Ferucarbotran (Resovist®, Bayer Healthcare), Ferumoxtran-10 (AMI-227 or Code-7227, Combidex®, AMAG Pharma; Sinerem®, Guerbet), NC100150 (Clariscan®, Nycomed,) and (VSOP C184, Ferropharm).

In some embodiments, the at least one coated iron oxide and/or at least one coated iron oxide particle is selected from the group consisting of Ferumoxytol, Ferumoxides, Ferucarbotran, Ferumoxtran-10, NC100150, VSOP C184, and combinations thereof.

In some embodiments, the iron oxide is superparamagnetic iron oxide (SPIO).

In some embodiments, the nanoparticles, probes, compositions, or articles of manufacture do not contain a targeting moiety. In some embodiments, the nanoparticles, probes, compositions, or articles of manufacture do not comprise a targeting moiety.

Polymers

In some embodiments, the at least one polymer is at least one biocompatible polymer.

In some embodiments, the at least one polymer is at least one polysaccharide.

In some embodiments, the at least one polymer is one selected from the group consisting of at least one dextran, at least one unfunctionalized dextran, at least one functionalized dextran, at least one unsubstituted dextran, at least one substituted dextran, and combinations thereof.

In some embodiments, the at least one polymer is selected from the group consisting of carboxymethyl dextran, at least one dextran, and combinations thereof.

In some embodiments, the at least one polymer is at least one selected from the group consisting of dextran, unfunctionalized dextran, functionalized dextran, unsubstituted dextran, substituted dextran, carboxymethyl dextran, unfunctionalized carboxymethyl dextran, functionalized carboxymethyl dextran, unsubstituted carboxymethyl dextran, substituted carboxymethyl dextran, and combinations thereof.

In some embodiments, the at least one polymer is poly(acrylic acid) (PAA).

Polysaccharides

In various embodiments, the at least one polymer is at least one polysaccharide.

In various embodiments, the at least one polysaccharide is selected from at least one dextran, at least one unfunctionalized dextran, at least one functionalized dextran, at least one unsubstituted dextran, at least one substituted dextran, and combinations thereof.

In some embodiments, the at least one polysaccharide is at least one selected from the group consisting of dextran, unfunctionalized dextran, functionalized dextran, unsubstituted dextran, substituted dextran, carboxymethyl dextran, unfunctionalized carboxymethyl dextran, functionalized carboxymethyl dextran, unsubstituted carboxymethyl dextran, substituted carboxymethyl dextran, and combinations thereof.

Dextrans

Dextrans are polysaccharides which have a linear backbone of a-linked d-glucopyranosyl repeating units. Three classes of dextrans can be differentiated by their structural features. The pyranose ring structure contains five carbon atoms and one oxygen atom. Class 1 dextrans contain the α(1→6)-linked d-glucopyranosyl backbone modified with small side chains of d-glucose branches with α(1→2), α(1→3), and α(1→4)-linkage. The class 1 dextrans vary in their molecular weight, spatial arrangement, type and degree of branching, and length of branch chains depending on the microbial producing strains and cultivation conditions. Isomaltose and isomaltotriose are oligosaccharides with the class 1 dextran backbone structure. Class 2 dextrans (alternans) contain a backbone structure of alternating α(1→3) and α(1→6)-linked d-glucopyranosyl units with α(1→3)-linked branches. Class 3 dextrans (mutans) have a backbone structure of consecutive α(1→3)-linked d-glucopyranosyl units with α(1→6)-linked branches.

In various embodiments, the at least one polymer is selected from the group consisting at least one dextran, at least one unfunctionalized dextran, at least one functionalized dextran, at least one unsubstituted dextran, at least one substituted dextran, and combinations thereof.

In various embodiments, the at least one polymer is selected from the group consisting of at least one dextran, carboxymethyl dextran, and combinations thereof.

In various embodiments, the at least one polymer is carboxymethyl dextran.

In some embodiments, the at least one dextran is selected from the group consisting of a class 1 dextran, a class 2 dextran, a class 3 dextran, and combinations thereof.

Probes

In various embodiments, the present invention provides a probe comprising a coated iron oxide nanoparticle; and at least one targeting moiety. In some embodiments, the at least one targeting moiety is attached to the coated iron oxide nanoparticle. In some embodiments, the coated iron oxide nanoparticle is selected from the group consisting of Ferumoxytol, Ferumoxides, Ferucarbotran, Ferumoxtran-10, NC100150, VSOP C184, and combinations thereof.

In various embodiments, the present invention provides a probe comprising a coated iron oxide nanoparticle. In some embodiments, the probe further comprises at least one targeting moiety. In some embodiments, the at least one targeting moiety is attached to the coated iron oxide nanoparticle. In some embodiments, the coated iron oxide nanoparticle is selected from the group consisting of Ferumoxytol, Ferumoxides, Ferucarbotran, Ferumoxtran-10, NC100150, VSOP C184, and combinations thereof.

In some embodiments, the probe further comprises at least one drug. In some embodiments, the probe further comprises at least one fluorescent dye. In some embodiments, the probe further comprises at least one drug, and at least one fluorescent dye.

In some embodiments, the probe is a multimodal probe. In some embodiments, the probe may be used for multimodal detection of a cancer in a subject. In some embodiments, the probe may be used for multimodal detection of a tumor in a subject. In some embodiments, the probe may be used for multimodal detection of a tumor margin of a tumor in a subject. In some embodiments, the probe may be used to deliver a drug for example to a cancer cell, cancer tissue, cancerous cell, cancerous tissue, or tumor.

In some embodiments, the probes of the present invention may be used to determine tumor concentration in a subject. In some embodiments, the probes of the present invention may be for dual visualization by magnetic resonance imaging (MRI) and fluorescence imaging. In some embodiments, the probes of the present invention may be used as markers during fluorescence image guided surgery for the intraoperative detection of tumor margins. In some embodiments, the probes of the present invention may be used to visualize drug delivery by magnetic resonance imaging and/or fluorescence imaging. In some embodiments, the fluorescence imaging is selected from the group consisting of near infrared fluorescence imaging, intraoperative fluorescence imaging, and combinations thereof.

Probes of the present invention may be administered to a subject (and thereby contacted with a tissue), or contacted with a tissue in vivo or in vitro. Thus, in some embodiments, the methods are applicable to both human therapy and veterinary applications, as well as research applications in vitro or within animal models.

In some embodiments, the probes of the present invention do not comprise a boron cluster. In some embodiments, the probes of the present invention do not contain a boron cluster. In some embodiments, the probes of the present invention do not comprise a compound comprising boron. In some embodiments, the probes of the present invention do not contain a compound comprising boron. In some embodiments, the probes of the present invention do not comprise boron. In some embodiments, the probes of the present invention do not contain boron.

In some embodiments, the probes of the present invention selectively target and/or bind to diseased tissue and/or diseased cells. In some embodiments, the probes of the present invention selectively target and/or bind to cancerous tissue, cancer tissue, cancer cells, tumor, tumor tissue, tumor cells, and combinations thereof.

In some embodiments, the probes of the present invention selectively targets and/or binds to diseased tissue and/or diseased cells compared to non-diseased tissue and/or non-diseased cells. In some embodiments, the probes of the present invention selectively targets and/or binds to cancerous tissue, cancer tissue, cancer cells, tumor, tumor tissue, tumor cells, and combinations thereof compared to healthy tissue, non-cancerous tissue, non-cancerous cells.

Targeting Moiety

The terms “targeting moiety” and “targeting agent” and “targeting ligand” are used interchangeably herein and are intended to mean any agent, such as for example a molecule, compound, or peptide, that serves to target or direct the nanoparticle or probe to a particular location or association (e.g., a specific binding event). Thus, for example, a targeting moiety may be used to target a molecule to a specific target protein or enzyme, or to a particular cellular location, or to a particular cell type, to selectively enhance accumulation of the nanoparticle or probe. For example, as discussed more fully herein, the nanoparticles and probes of the present invention include a targeting moiety to target the nanoparticles and probes to a specific cell type such as tumor cells, such as a transferrin moiety, since many tumor cells have significant transferrin receptors on their surfaces. Similarly, a targeting moiety may include components useful in targeting the nanoparticles or probes to a particular subcellular location. As will be appreciated by those of in the art, the localization of proteins within a cell is a simple method for increasing effective concentration. For example, shuttling a drug into the nucleus confines them to a smaller space thereby increasing concentration. The physiological target may simply be localized to a specific compartment, and the agent must be localized appropriately. More than one targeting moiety can be linked, connected, conjugated, attached, or otherwise associated with each nanoparticle or probe, and the target molecule for each targeting moiety can be the same or different.

The targeting moiety can function to target or direct the nanoparticle or probe to a particular location, cell type, tissue type, diseased cell, diseased tissue, or association. In general, the targeting moiety is directed against a target molecule. As will be appreciated by those in the art, the nanoparticles of the invention or probes of the invention are can be applied locally or systemically administered (e.g., injected intravenously).

In some embodiments, the targeting moiety may be used to either allow the internalization of the nanoparticle or probe to the cell cytoplasm or localize it to a particular cellular compartment, such as the nucleus. In some embodiments, the targeting moiety allows targeting of the nanoparticles of the present invention or probes of the present invention to a particular subcellular location, for example, the cytoplasm, Golgi, endoplasmic reticulum, nucleus, nucleoli, nuclear membrane, mitochondria, secretory vesicles, lysosome, and cellular membrane.

In some embodiments, the targeting moiety allows targeting of the nanoparticles of the present invention or probes of the present invention to extracellular locations (e.g., via a secretory signal). In some embodiments, the targeting moiety allows targeting of the nanoparticles of the present invention or probes of the present invention to a particular tissue or the surface of a cell (e.g., tumor tissue, cancer tissue, tumor cell, cancer cell). That is, in some embodiments, the nanoparticles of the present invention or probes of the present invention need not be taken up into the cytoplasm of a cell to be activated.

In some embodiments, the targeting moiety is selected the group consisting of heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep-2, modified angiopep-2, unsubstituted angiopep-2, substituted angiopep-2, unfunctionalized angiopep-2, functionalized angiopep-2, and combinations thereof.

In some embodiments, the targeting moiety is selected the group consisting of heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof. In some embodiments, the angiopep is selected from the group consisting of angiopep-1, angiopep-2, angiopep-5, angiopep-7, and combinations thereof. In some embodiments, the modified angiopep is selected from the group consisting of modified angiopep-1, modified angiopep-2, modified angiopep-5, modified angiopep-7, and combinations thereof. In some embodiments, the unsubstituted angiopep is selected from unsubstituted angiopep-1, unsubstituted angiopep-2, unsubstituted angiopep-5, unsubstituted angiopep-7, and combinations thereof. In some embodiments, the substituted angiopep is selected from the group consisting of substituted angiopep-1, substituted angiopep-2, substituted angiopep-5, unsubstituted angiopep-7, and combinations thereof. In some embodiments, unfunctionalized angiopep is selected from the group consisting of unfunctionalized angiopep-1, unfunctionalized angiopep-2, unfunctionalized angiopep-5, unfunctionalized angiopep-7, and combinations thereof. In some embodiments, functionalized angiopep is selected from the group consisting of functionalized angiopep-1, functionalized angiopep-2, functionalized angiopep-5, functionalized angiopep-7, and combinations thereof.

In some embodiments, the targeting moiety is selected from the group consisting of heptamethine carbocyanine dye, modified heptamethine carbocyanine dye, unsubstituted heptamethine carbocyanine dye, substituted heptamethine carbocyanine dye, unfunctionalized heptamethine carbocyanine dye, functionalized heptamethine carbocyanine dye, and combinations thereof.

In some embodiments, the targeting moiety is selected from the group consisting of heptamethine cyanine dye, modified heptamethine cyanine dye, unsubstituted heptamethine cyanine dye, substituted heptamethine cyanine dye, unfunctionalized heptamethine cyanine dye, functionalized heptamethine cyanine dye, and combinations thereof.

In some embodiments, the targeting moiety is a compound selected from the group consisting of Formula I and Formula II:

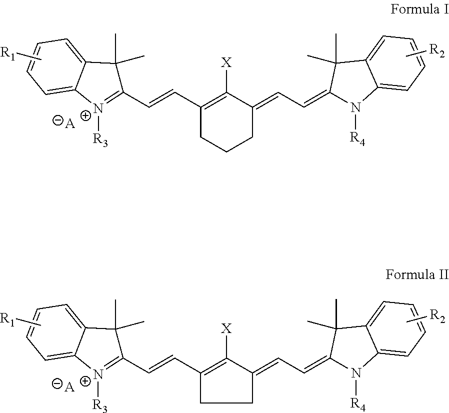

wherein R₁ and R₂ are each independently selected from the group consisting of hydrogen, sulfonato, an electron withdrawing group (EWG), an electron donating group (EDG), and are each independently attached at any of the aromatic ring positions;

R₃ and R₄ are independently selected from the group consisting of hydrogen, alkyl, aryl, aralkyl, alkylsulfonato, alkylcarboxy, alkylcarboxyl, alkylamino, ω-alkylaminium, ω-alkynyl, PEGyl, PEGylcarboxylate, ω-PEGylaminium, ω-acyl-NHRs, and ω-acyl-lysine, wherein R₅ is selected from the group consisting of hydrogen and alkyl;

X is selected from the group consisting of hydrogen, halogen, CN, Me, OH, 4-O-Ph-CH₂CH₂COOH, 4-O-Ph-NHR6, NHR₇, 4-S-Ph-NHRs, ω-iminoacyl-NHR9, and w-aminoacyl-lysine, wherein R₆, R₇, R₈, and R₉ are each independently selected from the group consisting of hydrogen and alkyl; and

counteranion A is selected from the group consisting of iodide, bromide, arylsulfonato, alkylsulfonato, tetrafluoroborate, chloride, and a pharmaceutically acceptable anion.

In some embodiments, R₃ and R₄ are not both hydrogen. In some embodiments, the counteranion A is not present.

In some embodiments, the targeting moiety targets glioma. In some embodiments, the targeting moiety is a glioma targeting moiety.

In some embodiments, the targeting moiety is not a component of a boron cluster. In some embodiments, the targeting moiety is not attached to a boron cluster. In some embodiments, the targeting moiety does not include a boron cluster. In some embodiments, the targeting moiety does not contain a boron cluster. In some embodiments, the targeting moiety does not comprise a boron cluster. In some embodiments, the targeting moiety does not comprise boron. In some embodiments, the targeting moiety does not contain boron.

In some embodiments, the targeting moiety is not a component of a compound comprising boron. In some embodiments, the targeting moiety is not attached to a compound comprising boron. In some embodiments, the targeting moiety does not include a compound comprising boron. In some embodiments, the targeting moiety does not contain a compound comprising boron. In some embodiments, the targeting moiety does not comprise a compound comprising boron.

The term “modified” refers to an alteration from an entity's normally occurring state. An entity can be modified by removing discrete chemical units or by adding discrete chemical units.

The terms “linked”, “joined”, “grafted”, “tethered”, “associated”, “attached”, “connected” and “conjugated” in the context of the nanoparticles of the invention or probes of the invention, are used interchangeably to refer to any method known in the art for functionally connecting moieties (e.g., targeting moieties) to the nanoparticles or components thereof or to the probes or components thereof or to the coated iron oxide nanoparticles or components thereof, including, without limitation, recombinant fusion, covalent bonding, non-covalent bonding, disulfide bonding, ionic bonding, hydrogen bonding, and electrostatic bonding.

In various embodiments, the at least one targeting moiety is attached to the at least one polymer or the ferumoxytol. In some embodiments, the at least one targeting moiety is linked to the at least one polymer or the ferumoxytol by at least one linkage. In some embodiments, the at least one targeting moiety is linked to the at least one polymer or the ferumoxytol by at least one linker. In some embodiments, the at least one targeting moiety is linked to the at least one carboxymethyl dextran. In some embodiments, the at least one targeting moiety is linked to the carboxymethyl dextran by at least one linkage. In some embodiments, the at least one targeting moiety is linked to the carboxymethyl dextran by at least one linker. In some embodiments, the at least one targeting moiety is attached to the shell. In some embodiments, the at least one targeting moiety is attached to the shell of the nanoparticle or probe. In some embodiments, the at least one targeting moiety is attached to the shell of the nanoparticle or probe by at least one linkage.

Non-limiting examples of linkages and/or linkers include a lysine linker, maleimide linker, maleimide-PEG-Amine linker, or combinations thereof. In some embodiments, the linkage and/or linker comprises at least one lysine. In some embodiments, the linkage and/or linker comprises at least one maleimide. In some embodiments, the linkage and/or linker comprises at least one maleimide-PEG-Amine.

In some embodiments, the targeting moiety selectively targets and/or binds to diseased tissue and/or diseased cells. In some embodiments, the targeting moiety selectively targets and/or binds to cancerous tissue, cancer tissue, cancer cells, tumor, tumor tissue, tumor cells, and combinations thereof.

In some embodiments, targeting moiety selectively targets and/or binds to diseased tissue and/or diseased cells compared to non-diseased tissue and/or non-diseased cells. In some embodiments, targeting moiety selectively targets and/or binds to cancerous tissue, cancer tissue, cancer cells, tumor, tumor tissue, tumor cells, and combinations thereof compared to healthy tissue, non-cancerous tissue, non-cancerous cells.

In some embodiments, the targeting moiety is an antibody that selectively targets and/or binds to diseased tissue and/or diseased cells compared to non-diseased tissue and/or non-diseased cells. In some embodiments, targeting moiety is an antibody that selectively targets and/or binds to cancerous tissue, cancer tissue, cancer cells, tumor, tumor tissue, tumor cells, and combinations thereof compared to healthy tissue, non-cancerous tissue, non-cancerous cells.

In some embodiments, the targeting moiety is a peptide that selectively targets and/or binds to diseased tissue and/or diseased cells compared to non-diseased tissue and/or non-diseased cells. In some embodiments, the targeting moiety is a peptide that selectively targets and/or binds to cancerous tissue, cancer tissue, cancer cells, tumor, tumor tissue, tumor cells, and combinations thereof compared to healthy tissue, non-cancerous tissue, non-cancerous cells.

Drugs

In various embodiments, the nanoparticles, compositions, articles of manufacture, and/or probes of the present invention may optionally further comprise at least one drug loaded into or encapsulated into or attached to the nanoparticles, compositions, articles of manufacture, and/or probes or components thereof. In various embodiments, the nanoparticle further comprises at least one drug. In various embodiments, the probe further comprises at least one drug.

In some embodiments, the at least one drug is encapsulated in the nanoparticle. In some embodiments, the at least one drug is encapsulated in the at least one polymer or in the ferumoxytol. In some embodiments, the at least one drug is linked to the at least one polymer or to the ferumoxytol. In some embodiments, the at least one drug is linked to the at least one polymer or to the ferumoxytol by at least one linkage. In some embodiments, the at least one drug is linked to the at least one polymer or to the ferumoxytol by at least one linker. In some embodiments, at least one drug is encapsulated in the carboxymethyl dextran. In some embodiments, the at least one drug is linked to the at least one carboxymethyl dextran. In some embodiments, the at least one drug is linked to the carboxymethyl dextran by at least one linkage. In some embodiments, the at least one drug is linked to the carboxymethyl dextran by at least one linker. In some embodiments, the at least one drug is encapsulated in the at least one coated iron oxide nanoparticle. In some embodiments, the at least one drug is linked to the at least one coated iron oxide nanoparticle. In some embodiments, the at least one drug is linked to the at least one coated iron oxide nanoparticle by at least one linkage. In some embodiments, the at least one drug is linked to the at least one coated iron oxide nanoparticle by at least one linker. In some embodiments, at least one drug is encapsulated in the shell.

Non-limiting examples of linkages and/or linkers include a lysine linker, maleimide linker, maleimide-PEG-Amine linker, or combinations thereof. In some embodiments, the linkage and/or linker comprises at least one lysine. In some embodiments, the linkage and/or linker comprises at least one maleimide. In some embodiments, the linkage and/or linker comprises at least one maleimide-PEG-Amine.

As used herein, the term “drug” refers to any agent capable of having a physiologic effect (e.g., a therapeutic or prophylactic effect) on a biosystem such as a prokaryotic or eukaryotic cells, or prokaryotic or eukaryotic tissue, or a subject (e.g., a patient), in vivo or in vitro, including, without limitation, chemotherapeutics, toxins, radiotherapeutics, radiosenitizing agents, gene therapy vectors, antisense nucleic acid constructs, transcription factor decoys, imaging agents, diagnostic agents, agents known to interact with an intracellular protein, polypeptides, and polynucleotides. Drugs that may be utilized in the nanoparticles or probes include any type of compound including antibacterial, antiviral, antifungal, or anti-cancer agents. In some embodiments, the drug may be modified to attach a polymerizable moiety. In some embodiments, the drug is water-insoluble, poorly water soluble, or water-soluble. In some embodiments, the drug is a solid or liquid. In some embodiments, the drug is a therapeutic agent. In some embodiments, the drug is not a therapeutic agent.

The drug need not be a therapeutic agent. For example, the drug may be cytotoxic to the local cells or tissue to which it is delivered but have an overall beneficial effect on the subject. Further, the drug may be a diagnostic agent with no direct therapeutic activity per se, such as a contrast agent for bioimaging.

As used herein, the term “therapeutic agent” refers to a compound used to treat or prevent a disease, disorder, or disease condition in a subject so as to provide a therapeutic benefit to the subject. In some embodiments, the therapeutic agent is administered to the subject in a therapeutically effective amount.

A description of various classes of drugs and diagnostic agents and a listing of species within each class can be found, for instance, in Martindale, The Extra Pharmacopoeia, Twenty-ninth Edition (The Pharmaceutical Press, London, 1989), which is incorporated herein by reference in its entirety. The drugs or diagnostic agents are commercially available and/or can be prepared by techniques known in the art.

Non-limiting examples of drugs include analgesics, anesthetics, anti-inflammatory agents, anthelmintics, anti-arrhythmic agents, antiasthma agents, antibiotics (including penicillins), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diagnostic agents, diagnostic imaging agents, diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, and xanthines.

Non-limiting examples of drugs include analgesics, anesthetics, anti-inflammatory agents, anthelmintics, anti-arrhythmic agents, antiasthma agents, antibiotics, anticancer agents, anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives, astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants, diagnostic agents, diagnostic imaging agents, diuretics, dopaminergics, free radical scavenging agents, growth factors, haemostatics, immunological agents, lipid regulating agents, muscle relaxants, proteins, peptides and polypeptides xanthines, alprazolam, amiodarone, amlodipine, astemizole, atenolol, azathioprine, azelatine, beclomethasone, β-lactam, budesonide, buprenorphine, butalbital, carbamazepine, carbidopa, cefotaxime, cephalexin, cholestyramine, ciprofloxacin, cisapride, cisplatin, clarithromycin, clonazepam, clozapine, cyclosporin, diazepam, diclofenac sodium, digoxin, dipyridamole, divalproex, dobutamine, doxazosin, enalapril, estradiol, etodolac, etoposide, famotidine, felodipine, fentanyl citrate, fexofenadine, finasteride, fluconazole, flunisolide, flurbiprofen, fluvoxamine, furosemide, glipizide, gliburide, ibuprofen, isosorbide dinitrate, isotretinoin, isradipine, itraconazole, ketoconazole, ketoprofen, lamotrigine, lansoprazole, loperamide, loratadine, lorazepam, lovastatin, medroxyprogesterone, mefenamic acid, methylprednisolone, midazolam, mometasone, nabumetone, naproxen, nicergoline, nifedipine, norfloxacin, omeprazole, paclitaxel, penicillin, phenytoin, piroxicam, quinapril, ramipril, risperidone, sertraline, simvastatin, steroids, taxol, terbinafine, terfenadine, triamcinolone, valproic acid, zolpidem, expectorants, mucolytics, hypnotics, neuroleptics, and a pharmaceutically acceptable salt of any of the foregoing.

Non-limiting examples of drugs include alprazolam, amiodarone, amlodipine, astemizole, atenolol, azathioprine, azelatine, beclomethasone, budesonide, buprenorphine, butalbital, carbamazepine, carbidopa, cefotaxime, cephalexin, cholestyramine, ciprofloxacin, cisapride, cisplatin, clarithromycin, clonazepam, clozapine, cyclosporin, diazepam, diclofenac sodium, digoxin, dipyridamole, divalproex, dobutamine, doxazosin, enalapril, estradiol, etodolac, etoposide, famotidine, felodipine, fentanyl citrate, fexofenadine, finasteride, fluconazole, fiunisolide, flurbiprofen, fluvoxamine, furosemide, glipizide, gliburide, ibuprofen, isosorbide dinitrate, isotretinoin, isradipine, itraconazole, ketoconazole, ketoprofen, lamotrigine, lansoprazole, loperamide, loratadine, lorazepam, lovastatin, medroxyprogesterone, mefenamic acid, methylprednisolone, midazolam, mometasone, nabumetone, naproxen, nicergoline, nifedipine, norfloxacin, omeprazole, paclitaxel, phenytoin, piroxicam, quinapril, ramipril, risperidone, sertraline, simvastatin, sulindac, terbinafine, terfenadine, triamcinolone, valproic acid, zolpidem, or pharmaceutically acceptable salts of any of the above-mentioned drugs.

Non-limiting examples of drugs include cisplatin, carboplatin, oxaliplatin, bortezomib, camptothecin, topotecan, irinotecan, temozolomide, doxorubicin, etoposide or pharmaceutically acceptable salts of any of the above-mentioned drugs.

In some embodiments, the drug is selected from the group consisting of docetaxel (DXT), paclitaxel (PXT), bortezomib (Bort), cobozentanib (cabo), brefeldin A (BFA), and combinations thereof. In some embodiments, the drug is selected from the group consisting of docetaxel (DXT), paclitaxel (PXT), bortezomib (Bort), cobozentanib (cabo), brefeldin A (BFA), and combinations thereof.

In some embodiments, the drug is not a boron cluster. In some embodiments, the drug is not a component of a boron cluster. In some embodiments, the drug does not include a boron cluster. In some embodiments, the drug does not contain a boron cluster. In some embodiments, the drug does not comprise a boron cluster. In some embodiments, the drug does not comprise boron. In some embodiments, the drug does not contain boron.

In some embodiments, the nanoparticles of the present invention can be used to deliver a drug that is cytotoxic to cancer cells or tumor cells. In some embodiments, the probes of the present invention can be used to deliver a drug that is cytotoxic to cancer cells or tumor cells.

The terms “linked”, “joined”, “grafted”, “tethered”, “associated”, “attached”, “connected” and “conjugated” in the context of the nanoparticles of the invention or the probes of the invention, are used interchangeably to refer to any method known in the art for functionally connecting drugs to the nanoparticles or components thereof or the probes or components thereof or the coated iron oxide nanoparticles or components thereof, including, without limitation, recombinant fusion, covalent bonding, non-covalent bonding, disulfide bonding, ionic bonding, hydrogen bonding, and electrostatic bonding.

Fluorescent Dyes

In various embodiments, the nanoparticles, compositions, articles of manufacture, and/or probes of the present invention may optionally further comprise at least one fluorescent dye loaded into or encapsulated into or attached to the nanoparticles, compositions, articles of manufacture, and/or probes or components thereof. In various embodiments, the nanoparticle further comprises at least one fluorescent dye.

In some embodiments, the at least one fluorescent dye is encapsulated in the nanoparticle. In some embodiments, the at least one fluorescent dye is encapsulated in the at least one polymer or in the ferumoxytol. In some embodiments, the at least one fluorescent dye is linked to the at least one polymer or to the ferumoxytol. In some embodiments, the at least one fluorescent dye is linked to the at least one polymer or to the ferumoxytol by at least one linkage. In some embodiments, the at least one fluorescent dye is linked to the at least one polymer or to the ferumoxytol by at least one linker. In some embodiments, at least one fluorescent dye is encapsulated in the carboxymethyl dextran. In some embodiments, the at least one fluorescent dye is linked to the at least one carboxymethyl dextran. In some embodiments, the at least one fluorescent dye is linked to the carboxymethyl dextran by at least one linkage. In some embodiments, the at least one fluorescent dye is linked to the carboxymethyl dextran by at least one linker. In some embodiments, at least one fluorescent dye is encapsulated in the shell.

In some embodiments, the at least one fluorescent dye is encapsulated in the at least one coated iron oxide nanoparticle. In some embodiments, the at least one fluorescent dye is linked to the at least one coated iron oxide nanoparticle. In some embodiments, the at least one fluorescent dye is linked to the at least one coated iron oxide nanoparticle by at least one linkage. In some embodiments, the at least one fluorescent dye is linked to the at least one coated iron oxide nanoparticle by at least one linker.

In some embodiments, the fluorescent dye is a near infrared dye. In some embodiments, the fluorescent dye is a near infrared fluorescent dye.

Non-limiting examples of fluorescent dyes include DiI, DiR, heptamethine cyanine (HMC), IR820, or combinations thereof.

Non-limiting examples of linkages and/or linkers include a lysine linker, maleimide linker, maleimide-PEG-Amine linker, or combinations thereof. In some embodiments, the linkage and/or linker comprises at least one lysine. In some embodiments, the linkage and/or linker comprises at least one maleimide. In some embodiments, the linkage and/or linker comprises at least one maleimide-PEG-Amine.

The terms “linked”, “joined”, “grafted”, “tethered”, “associated”, “attached”, “connected” and “conjugated” in the context of the nanoparticles of the invention or the probes of the invention, are used interchangeably to refer to any method known in the art for functionally connecting fluorescent dyes to the nanoparticles or components thereof or the probes or components thereof or the coated iron oxide nanoparticles or components thereof, including, without limitation, recombinant fusion, covalent bonding, non-covalent bonding, disulfide bonding, ionic bonding, hydrogen bonding, and electrostatic bonding.

In some embodiments, the fluorescent dye is not a boron cluster. In some embodiments, the fluorescent dye is not a component of a boron cluster. In some embodiments, the fluorescent dye does not include a boron cluster. In some embodiments, the fluorescent dye does not contain a boron cluster. In some embodiments, the fluorescent dye does not comprise a boron cluster. In some embodiments, the fluorescent dye does not comprise boron. In some embodiments, the fluorescent dye does not contain boron.

Pharmaceutical Compositions

In various embodiments the present invention also provides the nanoparticles of the present invention in the form of various pharmaceutical formulations. The present invention also provides the probes of the present invention in the form of various pharmaceutical formulations. These pharmaceutical compositions may be used for example for detecting, diagnosing, treating, detecting and treating, diagnosing and treating, reducing the severity of and/or slowing the progression of a disease, disorder, or disease condition in a subject. In accordance with the invention, the disease, disorder, or disease condition can be a cancer.

In various embodiments, the present invention provides a pharmaceutical composition comprising at least one nanoparticle described herein. In another embodiment, the present invention provides a pharmaceutical composition comprising at least two nanoparticles described herein. In still another embodiment, the present invention provides a pharmaceutical composition comprising a plurality of nanoparticles described herein. In accordance with the present invention, the nanoparticles comprise a targeting moiety linked, connected, or conjugated thereto. In various embodiments, the pharmaceutical compositions also exhibit minimal toxicity when administered to a mammal.

In various embodiments, the present invention provides a pharmaceutical composition comprising at least one probe described herein. In another embodiment, the present invention provides a pharmaceutical composition comprising at least two probes described herein. In still another embodiment, the present invention provides a pharmaceutical composition comprising a plurality of probes described herein. In accordance with the present invention, the probes comprise a targeting moiety linked, connected, or conjugated thereto or to a component thereof. In various embodiments, the pharmaceutical compositions also exhibit minimal toxicity when administered to a mammal.

In various embodiments, the pharmaceutical compositions according to the invention can contain any pharmaceutically acceptable excipient. “Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients may be solid, liquid, semisolid, or, in the case of an aerosol composition, gaseous. Examples of excipients include but are not limited to starches, sugars, microcrystalline cellulose, diluents, granulating agents, lubricants, binders, disintegrating agents, wetting agents, emulsifiers, coloring agents, release agents, coating agents, sweetening agents, flavoring agents, perfuming agents, preservatives, antioxidants, plasticizers, gelling agents, thickeners, hardeners, setting agents, suspending agents, surfactants, humectants, carriers, stabilizers, and combinations thereof.

In various embodiments, the pharmaceutical compositions according to the invention may be formulated for delivery via any route of administration. “Route of administration” may refer to any administration pathway known in the art, including but not limited to aerosol, nasal, oral, transmucosal, transdermal, parenteral, enteral, topical or local. “Parenteral” refers to a route of administration that is generally associated with injection, including intraorbital, infusion, intraarterial, intracapsular, intracardiac, intradermal, intramuscular, intraperitoneal, intrapulmonary, intraspinal, intrasternal, intrathecal, intrauterine, intravenous, subarachnoid, subcapsular, subcutaneous, transmucosal, or transtracheal. Via the parenteral route, the compositions may be in the form of solutions or suspensions for infusion or for injection, or as lyophilized powders. Via the parenteral route, the compositions may be in the form of solutions or suspensions for infusion or for injection. Via the enteral route, the pharmaceutical compositions can be in the form of tablets, gel capsules, sugar-coated tablets, syrups, suspensions, solutions, powders, granules, emulsions, microspheres or nanospheres or lipid vesicles or polymer vesicles allowing controlled release. Typically, the compositions are administered by injection. Methods for these administrations are known to one skilled in the art. In certain embodiments, the pharmaceutical composition is formulated for intravascular, intravenous, intraarterial, intratumoral, intramuscular, subcutaneous, intranasal, intraperitoneal, or oral administration.

In various embodiments, the pharmaceutical compositions according to the invention can contain any pharmaceutically acceptable carrier. “Pharmaceutically acceptable carrier” as used herein refers to a pharmaceutically acceptable material, composition, or vehicle that is involved in carrying or transporting a compound of interest from one tissue, organ, or portion of the body to another tissue, organ, or portion of the body. For example, the carrier may be a liquid or solid filler, diluent, excipient, solvent, or encapsulating material, or a combination thereof. Each component of the carrier must be “pharmaceutically acceptable” in that it must be compatible with the other ingredients of the formulation. It must also be suitable for use in contact with any tissues or organs with which it may come in contact, meaning that it must not carry a risk of toxicity, irritation, allergic response, immunogenicity, or any other complication that excessively outweighs its therapeutic benefits.

The pharmaceutical compositions according to the invention can also be encapsulated, tableted or prepared in an emulsion or syrup for oral administration. Pharmaceutically acceptable solid or liquid carriers may be added to enhance or stabilize the composition, or to facilitate preparation of the composition. Liquid carriers include syrup, peanut oil, olive oil, glycerin, saline, alcohols and water. Solid carriers include starch, lactose, calcium sulfate, dihydrate, terra alba, magnesium stearate or stearic acid, talc, pectin, acacia, agar or gelatin. The carrier may also include a sustained release material such as glyceryl monostearate or glyceryl distearate, alone or with a wax.

The pharmaceutical preparations are made following the conventional techniques of pharmacy involving milling, mixing, granulation, and compressing, when necessary, for tablet forms; or milling, mixing and filling for hard gelatin capsule forms. When a liquid carrier is used, the preparation will be in the form of a syrup, elixir, emulsion or an aqueous or non-aqueous suspension. Such a liquid formulation may be administered directly p.o. or filled into a soft gelatin capsule.

The pharmaceutical compositions according to the invention may be delivered in a therapeutically effective amount. The precise therapeutically effective amount is that amount of the composition that will yield the most effective results in terms of efficacy of treatment in a given subject. This amount will vary depending upon a variety of factors, including but not limited to the characteristics of the therapeutic compound (including activity, pharmacokinetics, pharmacodynamics, and bioavailability), the physiological condition of the subject (including age, sex, disease type and stage, general physical condition, responsiveness to a given dosage, and type of medication), the nature of the pharmaceutically acceptable carrier or carriers in the formulation, and the route of administration. One skilled in the clinical and pharmacological arts will be able to determine a therapeutically effective amount through routine experimentation, for instance, by monitoring a subject's response to administration of a compound and adjusting the dosage accordingly. For additional guidance, see Remington: The Science and Practice of Pharmacy (Gennaro ed. 20th edition, Williams & Wilkins PA, USA) (2000).

Before administration to patients, formulants may be added to the composition. A liquid formulation may be preferred. For example, these formulants may include oils, polymers, vitamins, carbohydrates, amino acids, salts, buffers, albumin, surfactants, bulking agents or combinations thereof.

Carbohydrate formulants include sugar or sugar alcohols such as monosaccharides, disaccharides, or polysaccharides, or water soluble glucans. The saccharides or glucans can include fructose, dextrose, lactose, glucose, mannose, sorbose, xylose, maltose, sucrose, dextran, pullulan, dextrin, alpha and beta cyclodextrin, soluble starch, hydroxethyl starch and carboxymethylcellulose, or mixtures thereof. “Sugar alcohol” is defined as a C4 to C8 hydrocarbon having an —OH group and includes galactitol, inositol, mannitol, xylitol, sorbitol, glycerol, and arabitol. These sugars or sugar alcohols mentioned above may be used individually or in combination. There is no fixed limit to amount used as long as the sugar or sugar alcohol is soluble in the aqueous preparation. In one embodiment, the sugar or sugar alcohol concentration is between 1.0 w/v % and 7.0 w/v %, more preferable between 2.0 and 6.0 w/v %.

Amino acids formulants include levorotary (L) forms of carnitine, arginine, and betaine; however, other amino acids may be added.

In some embodiments, polymers as formulants include polyvinylpyrrolidone (PVP) with an average molecular weight between 2,000 and 3,000, or polyethylene glycol (PEG) with an average molecular weight between 3,000 and 5,000.

It is also preferred to use a buffer in the composition to minimize pH changes in the solution before lyophilization or after reconstitution. Most any physiological buffer may be used including but not limited to citrate, phosphate, succinate, and glutamate buffers or mixtures thereof. In some embodiments, the concentration is from 0.01 to 0.3 molar. Surfactants that can be added to the formulation are shown in EP Nos. 270,799 and 268,110.

After the liquid pharmaceutical composition is prepared, it may be lyophilized to prevent degradation and to preserve sterility. Methods for lyophilizing liquid compositions are known to those of ordinary skill in the art. Just prior to use, the composition may be reconstituted with a sterile diluent (Ringer's solution, distilled water, or sterile saline, for example) which may include additional ingredients. Upon reconstitution, the composition is administered to subjects using those methods that are known to those skilled in the art.

The compositions of the invention may be sterilized by conventional, well-known sterilization techniques. The resulting solutions may be packaged for use or filtered under aseptic conditions and lyophilized, the lyophilized preparation being combined with a sterile solution prior to administration. The compositions may contain pharmaceutically-acceptable auxiliary substances as required to approximate physiological conditions, such as pH adjusting and buffering agents, tonicity adjusting agents and the like, for example, sodium acetate, sodium lactate, sodium chloride, potassium chloride, calcium chloride, and stabilizers (e.g., 1-20% maltose, etc.).

In some embodiments, the pharmaceutical composition does not include a boron cluster. In some embodiments, the pharmaceutical composition does not contain a boron cluster. In some embodiments, the pharmaceutical composition does not comprise a boron cluster. In some embodiments, the pharmaceutical composition does not comprise boron. In some embodiments, the pharmaceutical composition does not contain boron.

Kits

In various embodiments, the present invention provides a kit for diagnosing, detecting, treating, detecting and treating, diagnosing and treating, reducing the severity of and/or slowing the progression of a disease, disorder, or disease condition in a subject. The kit comprises: a quantity of the at least one nanoparticle of the present invention described herein; and instructions for using the nanoparticles to detect, diagnose, treat, detect and treat, diagnose and treat, reduce the severity of and/or slow the progression of the disease, disorder, or disease condition in the subject. In some embodiments of the present invention, the nanoparticle comprises at least one drug. In some embodiments, the nanoparticle comprises at least one fluorescent dye. In some embodiments, the nanoparticle comprises at least one drug and at least one fluorescent dye.

In various embodiments, the present invention provides a kit for diagnosing, detecting, treating, detecting and treating, diagnosing and treating, reducing the severity of and/or slowing the progression of a disease, disorder, or disease condition in a subject. The kit comprises: a quantity of the at least one probe of the present invention described herein; and instructions for using the probes to detect, diagnose, treat, detect and treat, diagnose and treat, reduce the severity of and/or slow the progression of the disease, disorder, or disease condition in the subject. In some embodiments of the present invention, the probe comprises at least one drug. In some embodiments, the probe comprises at least one fluorescent dye. In some embodiments, the probe comprises at least one drug and at least one fluorescent dye.

The kit is an assemblage of materials or components, including at least one of the inventive compositions and/or nanoparticles and/or probes. The exact nature of the components configured in the inventive kit depends on its intended purpose. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to affect a desired outcome. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example, the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well-known methods, preferably to provide a sterile, contaminant-free environment. As used herein, the term “package” refers to a suitable solid matrix or material such as glass, plastic, paper, foil, and the like, capable of holding the individual kit components. Thus, for example, a package can be a glass vial used to contain suitable quantities of a composition as described herein. The packaging material generally has an external label which indicates the contents and/or purpose of the kit and/or its components.

Methods for Detecting Nanoparticles

In various embodiments, the present invention provides a method for detecting at least one nanoparticle in a subject, comprising: administering the at least one nanoparticle to the subject; and detecting the at least one nanoparticle in the subject by an imaging method. In some embodiments, the at least one nanoparticle is a nanoparticle of the present invention.

In various embodiments the present invention provides a method for detecting at least one nanoparticle in a subject, comprising: administering the at least one nanoparticle to the subject, thereby contacting a tissue of the subject with the at least one nanoparticle such that the at least one nanoparticle binds to the tissue; and detecting the at least one nanoparticle bound to the tissue by an imaging method. In some embodiments, the at least one nanoparticle is a nanoparticle of the present invention.

In some embodiments, the imaging method is selected from the group consisting of magnetic resonance imaging, fluorescence imaging, and combinations thereof. In some embodiments, the fluorescence imaging is near infrared fluorescence imaging. In some embodiments, the fluorescence imaging is selected from the group consisting of near infrared fluorescence imaging, intraoperative fluorescence imaging, and combinations thereof.

In some embodiments, the imaging method comprises operating an imaging scanner.

In some embodiments, the imaging method comprises operating an imaging machine. In some embodiments, the imaging method comprises operating imaging equipment.

In some embodiments, the magnetic resonance imaging comprises operating a magnetic resonance imaging scanner. In some embodiments, the magnetic resonance imaging comprises operating a magnetic resonance imaging machine. In some embodiments, the magnetic resonance imaging comprises operating a magnetic resonance imaging instrument.

In some embodiments, the fluorescence imaging comprises operating a fluorescence imaging scanner. In some embodiments, the fluorescence imaging comprises operating a fluorescence imaging machine. In some embodiments, the fluorescence imaging comprises operating a fluorescence imaging instrument.

In some embodiments, the near infrared fluorescence imaging comprises operating a near infrared fluorescence imaging scanner or a fluorescence imaging scanner. In some embodiments, the near infrared fluorescence imaging comprises operating a near infrared fluorescence imaging machine or a fluorescence imaging machine. In some embodiments, the near infrared fluorescence imaging comprises operating a near infrared fluorescence imaging instrument or a fluorescence imaging instrument.

In some embodiments, the intraoperative fluorescence imaging comprises operating an intraoperative fluorescence imaging scanner or a fluorescence imaging scanner. In some embodiments, the intraoperative fluorescence imaging comprises operating an intraoperative fluorescence imaging machine or a fluorescence imaging machine. In some embodiments, the intraoperative fluorescence imaging comprises operating an intraoperative fluorescence imaging instrument or a fluorescence imaging instrument.

In some embodiments, the tissue is selected from the group consisting of cancerous tissue, cancer tissue, tumor, tumor tissue, and combinations thereof. In some embodiments, the tissue is selected from the group consisting of non-cancerous tissue, healthy tissue, normal tissue, cancerous tissue, cancer tissue, tumor, tumor tissue, and combinations thereof. In some embodiments, the tissue is selected from the group consisting of non-diseased tissue, healthy tissue, normal tissue, diseased tissue, and combinations thereof.

Methods for Detecting Probes

In various embodiments, the present invention provides a method for detecting at least one probe in a subject, comprising: administering the at least one probe to the subject; and detecting the at least one probe in the subject by an imaging method. In some embodiments, the at least one probe is a probe of the present invention.

In various embodiments the present invention provides a method for detecting at least one probe in a subject, comprising: administering the at least one probe to the subject, thereby contacting a tissue of the subject with the at least one probe such that the at least one probe binds to the tissue; and detecting the at least one probe bound to the tissue by an imaging method. In some embodiments, the at least one probe is a probe of the present invention.

In some embodiments, the imaging method is selected from the group consisting of magnetic resonance imaging, fluorescence imaging, and combinations thereof. In some embodiments, the fluorescence imaging is near infrared fluorescence imaging. In some embodiments, the fluorescence imaging is selected from the group consisting of near infrared fluorescence imaging, intraoperative fluorescence imaging, and combinations thereof.

In some embodiments, the tissue is selected from the group consisting of cancerous tissue, cancer tissue, tumor, tumor tissue, and combinations thereof.

Methods for Diagnosing Cancer

In various embodiments, the present invention provides a method for diagnosing a cancer in a subject, comprising: administering an effective amount of at least one nanoparticle to the subject, thereby contacting a tissue of the subject with the at least one nanoparticle such that the at least one nanoparticle binds to the tissue; and detecting the at least one nanoparticle bound to the tissue, wherein the presence of the at least one nanoparticle bound to the tissue is indicative of the cancer in the subject. In some embodiments, the at least one nanoparticle is a nanoparticle of the present invention.

In various embodiments, the present invention provides a method for diagnosing a cancer in a subject, comprising: administering an effective amount of at least one probe to the subject, thereby contacting a tissue of the subject with the at least one probe such that the at least one probe binds to the tissue; and detecting the at least one probe bound to the tissue, wherein the presence of the at least one probe bound to the tissue is indicative of the cancer in the subject. In some embodiments, the at least one probe is a probe of the present invention.

In various embodiments, the present invention provides a method for diagnosing cancer in a subject, comprising: providing at least one nanoparticle, wherein the at least one nanoparticle comprises: a core, wherein the core comprises at least one iron oxide; a shell surrounding the core, wherein the shell comprises at least one polymer; and at least one targeting moiety attached to the shell; administering an effective amount of the at least one nanoparticle to the subject, thereby contacting a tissue of the subject with the at least one nanoparticle, wherein the tissue is selected from the group consisting of cancerous tissue, non-cancerous tissue, and combinations thereof, and wherein the nanoparticle selectively binds to the cancerous tissue; and detecting the at least one nanoparticle bound to the cancerous tissue, wherein the presence of the at least one nanoparticle bound to the cancerous tissue is a diagnosis of the cancer in the subject.

Methods for Detecting Cancer

In various embodiments, the present invention provides a method for detecting a cancer in a subject, comprising: administering an effective amount of at least one nanoparticle to the subject, thereby contacting a tissue of the subject with the at least one nanoparticle such that the at least one nanoparticle binds to the tissue; and detecting the at least one nanoparticle bound to the tissue, wherein the presence of the at least one nanoparticle bound to the tissue is indicative of the cancer in the subject. In some embodiments, the at least one nanoparticle is a nanoparticle of the present invention.

In various embodiments, the present invention provides a method for detecting a cancer in a subject, comprising: administering an effective amount of at least one probe to the subject, thereby contacting a tissue of the subject with the at least one probe such that the at least one probe binds to the tissue; and detecting the at least one probe bound to the tissue, wherein the presence of the at least one probe bound to the tissue is indicative of the cancer in the subject. In some embodiments, the at least one probe is a probe of the present invention.

In some embodiments, the tissue is selected from the group consisting of cancerous tissue, cancer tissue, tumor, tumor tissue, and combinations thereof.

In various embodiments, the present invention provides a method for detecting cancer in a subject, comprising: providing at least one nanoparticle, wherein the at least one nanoparticle comprises: a core, wherein the core comprises at least one iron oxide; a shell surrounding the core, wherein the shell comprises at least one polymer; and at least one targeting moiety attached to the shell; administering an effective amount of the at least one nanoparticle to the subject, thereby contacting a tissue of the subject with the at least one nanoparticle, wherein the tissue is selected from the group consisting of cancerous tissue, non-cancerous tissue, and combinations thereof, and wherein the nanoparticle selectively binds to the cancerous tissue; and detecting the at least one nanoparticle bound to the cancerous tissue, wherein the presence of the at least one nanoparticle bound to the cancerous tissue is indicative of the cancer in the subject.

Methods for Treating Cancer

In various embodiments, the present invention provides a method for treating a cancer in a subject, comprising: administering an effective amount of at least one nanoparticle to the subject, thereby contacting a tissue of the subject with the at least one nanoparticle such that the at least one nanoparticle binds to the tissue, wherein the at least one nanoparticle comprises at least one drug; detecting the at least one nanoparticle bound to the tissue, wherein the presence of the at least one nanoparticle bound to the tissue is indicative of the cancer in the subject; and delivering the at least one drug to the tissue thereby treating the cancer in the subject. In some embodiments, the at least one nanoparticle is a nanoparticle of the present invention.

In various embodiments, the present invention provides a method for treating a cancer in a subject, comprising: administering an effective amount of at least one probe to the subject, thereby contacting a tissue of the subject with the at least one probe such that the at least one probe binds to the tissue; detecting the at least one probe bound to the tissue, wherein the presence of the at least one probe bound to the tissue is indicative of the cancer in the subject; and delivering the drug to the tissue thereby treating the cancer in the subject. In some embodiments, the tissue is selected from the group consisting of cancerous tissue, cancer tissue, tumor, tumor tissue, and combinations thereof.

In various embodiments, the present invention provides a method for treating, reducing the severity of and/or slowing the progression of cancer in a subject, comprising: providing at least one nanoparticle, wherein the at least one nanoparticle comprises: a core, wherein the core comprises at least one iron oxide; a shell surrounding the core, wherein the shell comprises at least one polymer; and at least one targeting moiety attached to the shell; and administering a therapeutically effective amount of the at least one nanoparticle to the subject, thereby contacting a tissue of the subject with the at least one nanoparticle, wherein the tissue is selected from the group consisting of cancerous tissue, non-cancerous tissue, and combinations thereof, and wherein the nanoparticle selectively binds to the cancerous tissue, thereby treating, reducing the severity of and/or slowing the progression of the cancer in the subject. In some embodiments, the nanoparticle further comprises at least one drug. In some embodiments, the method further comprises, delivering a drug to the cancerous tissue so as to treat, reduce the severity of and/or slow the progression of the cancer in the subject.

Methods for Diagnosing and Treating Cancer

In various embodiments, the present invention provides a method for diagnosing and treating a cancer in a subject, comprising: administering an effective amount of at least one nanoparticle to the subject, thereby contacting a tissue of the subject with the at least one nanoparticle such that the at least one nanoparticle binds to the tissue, wherein the at least one nanoparticle comprises at least one drug; detecting the nanoparticle bound to the tissue, wherein the presence of the at least one nanoparticle bound to the tissue is indicative of the cancer in the subject; and delivering the drug to the tissue thereby treating the cancer in the subject. In some embodiments, the at least one nanoparticle is a nanoparticle of the present invention.

In various embodiments, the present invention provides a method for diagnosing and treating a cancer in a subject, comprising: administering an effective amount of at least one probe to the subject, thereby contacting a tissue of the subject with the at least one probe such that the at least one probe binds to the tissue; detecting the at least one probe bound to the tissue, wherein the presence of the at least one probe bound to the tissue is indicative of the cancer in the subject; and delivering the drug to the tissue thereby treating the cancer in the subject. In some embodiments, the at least one probe is a probe of the present invention.

In some embodiments, the tissue is selected from the group consisting of cancerous tissue, cancer tissue, tumor, tumor tissue, and combinations thereof.

Methods for Detecting and Treating Cancer

In various embodiments, the present invention provides a method for detecting and treating a cancer in a subject, comprising: administering an effective amount of at least one nanoparticle to the subject, thereby contacting a tissue of the subject with the nanoparticle such that the nanoparticle binds to the tissue, wherein the at least one nanoparticle comprises at least one drug; detecting the nanoparticle bound to the tissue, wherein the presence of the nanoparticle bound to the tissue is indicative of the cancer in the subject; and delivering the drug to the tissue thereby treating the cancer in the subject. In some embodiments, the at least one nanoparticle is a nanoparticle of the present invention.

In various embodiments, the present invention provides a method for detecting and treating a cancer in a subject, comprising: administering an effective amount of at least one probe to the subject, thereby contacting a tissue of the subject with the at least one probe such that the at least one probe binds to the tissue; detecting the at least one probe bound to the tissue, wherein the presence of the at least one probe bound to the tissue is indicative of the cancer in the subject; and delivering the drug to the tissue thereby treating the cancer in the subject. In some embodiments, the at least one probe is a probe of the present invention.

In some embodiments, the tissue is selected from the group consisting of cancerous tissue, cancer tissue, tumor, tumor tissue, and combinations thereof.

Methods for Reducing the Severity of and/or Slowing the Progression of Cancer

In various embodiments, the present invention provides a method of reducing the severity of and/or slowing the progression of a cancer in a subject, administering an effective amount of at least one nanoparticle to the subject, thereby contacting a tissue of the subject with the nanoparticle such that the nanoparticle binds to the tissue, wherein the at least one nanoparticle comprises at least one drug; detecting the nanoparticle bound to the tissue, wherein the presence of the nanoparticle bound to the tissue is indicative of the cancer in the subject; and delivering the drug to the tissue thereby reducing the severity of and/or slowing the progression of the cancer in the subject. In some embodiments, the at least one nanoparticle is a nanoparticle of the present invention.

In various embodiments, the present invention provides a method of reducing the severity of and/or slowing the progression of a cancer in a subject, administering an effective amount of at least one probe to the subject, thereby contacting a tissue of the subject with the probe such that the probe binds to the tissue, wherein the at least one probe comprises at least one drug; detecting the probe bound to the tissue, wherein the presence of the probe bound to the tissue is indicative of the cancer in the subject; and delivering the drug to the tissue thereby reducing the severity of and/or slowing the progression of the cancer in the subject. In some embodiments, the at least one probe is a probe of the present invention.

In some embodiments, the tissue is selected from the group consisting of cancerous tissue, cancer tissue, tumor, tumor tissue, and combinations thereof.

Methods for Detecting a Tumor

In various embodiments, the present invention provides a method for detecting a tumor in a subject, comprising: administering an effective amount of at least one nanoparticle to the subject, thereby contacting a tumor present in the subject with the at least one nanoparticle such that the at least one nanoparticle binds to the tumor; and detecting the at least one nanoparticle bound to the tumor, wherein the presence of the at least one nanoparticle bound to the tumor is indicative of the presence of the tumor in the subject. In some embodiments, the at least one nanoparticle is a nanoparticle of the present invention.

In various embodiments, the present invention provides a method for detecting a tumor in a subject, comprising: administering an effective amount of at least one probe to the subject, thereby contacting a tumor present in the subject with the at least one probe such that the at least one probe binds to the tumor; and detecting the at least one probe bound to the tumor, wherein the presence of the at least one probe bound to the tumor is indicative of the presence of the tumor in the subject. In some embodiments, the at least one probe is a probe of the present invention.

Methods for Detecting a Tumor Margin of a Tumor

In various embodiments the present invention provides a method for detecting a tumor margin in a subject, comprising: administering an effective amount of at least one nanoparticle to the subject, thereby contacting a tumor present in the subject with the at least one nanoparticle such that the at least one nanoparticle binds to the tumor; and detecting the at least one nanoparticle bound to the tumor, wherein the presence of the at least one nanoparticle bound to the tumor is indicative of the tumor margin of the tumor in the subject. In some embodiments, the at least one nanoparticle is a nanoparticle of the present invention. In some embodiments, the at least one nanoparticle is detected using magnetic resonance imaging. In some embodiments, the at least one nanoparticle is detected using fluorescence imaging. In some embodiments, the at least one nanoparticle is detected using magnetic resonance imaging and fluorescence imaging. In some embodiments, the fluorescence imaging is near infrared fluorescence imaging. In some embodiments, the fluorescence imaging is selected from the group consisting of near infrared fluorescence imaging, intraoperative fluorescence imaging, and combinations thereof. In some embodiments, the method further comprises detecting and/or identifying the tumor margin before surgery. In some embodiments, the method further comprises detecting and/or identifying the tumor margin during surgery.

In various embodiments the present invention provides a method for detecting a tumor margin in a subject, comprising: administering an effective amount of at least one probe to the subject, thereby contacting a tumor present in the subject with the at least one probe such that the at least one probe binds to the tumor; and detecting the at least one probe bound to the tumor, wherein the presence of the at least one probe bound to the tumor is indicative of the tumor margin of the tumor in the subject. In some embodiments, the at least one probe is a probe of the present invention. In some embodiments, the at least one probe is detected using magnetic resonance imaging. In some embodiments, the at least one probe is detected using fluorescence imaging. In some embodiments, the at least one probe is detected using magnetic resonance imaging and fluorescence imaging. In some embodiments, the fluorescence imaging is near infrared fluorescence imaging. In some embodiments, the fluorescence imaging is selected from the group consisting of near infrared fluorescence imaging, intraoperative fluorescence imaging, and combinations thereof. In some embodiments, the method further comprises detecting and/or identifying the tumor margin before surgery. In some embodiments, the method further comprises detecting and/or identifying the tumor margin during surgery.

Treatments/Therapies and Additional Treatments/Therapies

In some embodiments, the method further comprises treating the subject with a therapy or treatment and/or administering a therapy or treatment to the subject and/or selecting a therapy or treatment for the subject and/or providing a therapy or treatment to the subject. In some embodiments, the treatment is a treatment for cancer. In some embodiments, the treatment is a cancer treatment. In some embodiments, the therapy is a therapy for cancer. In some embodiments, the therapy is a cancer therapy.

In some embodiments, the methods of the present invention may optionally further comprise simultaneously or sequentially administering a therapy or treatment to the subject. Non-limiting examples of treatments and therapies include pharmacological therapy, biological therapy, cell therapy, gene therapy, chemotherapy, radiation therapy, hormonal therapy, surgery, immunotherapy, or combinations thereof.

In some embodiments, the method further comprises treating the subject with an additional therapy or treatment and/or administering an additional therapy or treatment to the subject and/or selecting an additional therapy or treatment for the subject and/or providing an additional therapy or treatment to the subject. In some embodiments, the additional treatment is a treatment for cancer. In some embodiments, the additional treatment is a cancer treatment. In some embodiments, the additional therapy is a therapy for cancer. In some embodiments, the additional therapy is a cancer therapy.

In some embodiments, the methods of the present invention may optionally further comprise simultaneously or sequentially administering an additional therapy or treatment to the subject. Non-limiting examples of additional treatments and therapies include pharmacological therapy, biological therapy, cell therapy, gene therapy, chemotherapy, radiation therapy, hormonal therapy, surgery, immunotherapy, or combinations thereof.

In some embodiments, chemotherapy may comprise the use of chemotherapeutic agents. In some embodiments, chemotherapeutic agents may be selected from any one or more of cytotoxic antibiotics, antimetabolites, anti-mitotic agents, alkylating agents, arsenic compounds, DNA topoisomerase inhibitors, taxanes, nucleoside analogues, plant alkaloids, and toxins; and synthetic derivatives thereof. Exemplary compounds include, but are not limited to, alkylating agents: treosulfan, and trofosfamide; plant alkaloids: vinblastine, paclitaxel, docetaxol; DNA topoisomerase inhibitors: doxorubicin, epirubicin, etoposide, camptothecin, topotecan, irinotecan, teniposide, crisnatol, and mitomycin; anti-folates: methotrexate, mycophenolic acid, and hydroxyurea; pyrimidine analogs: 5-fluorouracil, doxifluridine, and cytosine arabinoside; purine analogs: mercaptopurine and thioguanine; DNA antimetabolites: 2′-deoxy-5-fluorouridine, aphidicolin glycinate, and pyrazoloimidazole; and antimitotic agents: halichondrin, colchicine, and rhizoxin. Compositions comprising one or more chemotherapeutic agents (e.g., FLAG, CHOP) may also be used. FLAG comprises fludarabine, cytosine arabinoside (Ara-C) and G-CSF. CHOP comprises cyclophosphamide, vincristine, doxorubicin, and prednisone. In another embodiment, PARP (e.g., PARP-1 and/or PARP-2) inhibitors are used and such inhibitors are well known in the art (e.g., Olaparib, ABT-888, BSI-201, BGP-15 (N-Gene Research Laboratories, Inc.); INO-1001 (Inotek Pharmaceuticals Inc.); PJ34 (Soriano et al., 2001; Pacher et al., 2002b); 3-aminobenzamide (Trevigen); 4-amino-1,8-naphthalimide; (Trevigen); 6(5H)-phenanthridinone (Trevigen); benzamide (U.S. Pat. Re. 36,397); and NU1025 (Bowman et al.).

In various embodiments, radiation therapy can be ionizing radiation. Radiation therapy can also be gamma rays, X-rays, or proton beams. Examples of radiation therapy include, but are not limited to, external-beam radiation therapy, interstitial implantation of radioisotopes (1-125, palladium, iridium), radioisotopes such as strontium-89, thoracic radiation therapy, intraperitoneal P-32 radiation therapy, and/or total abdominal and pelvic radiation therapy. For a general overview of radiation therapy, see Hellman, Chapter 16: Principles of Cancer Management: Radiation Therapy, 6th edition, 2001, DeVita et al., eds., J. B. Lippencott Company, Philadelphia. The radiation therapy can be administered as external beam radiation or tele-therapy wherein the radiation is directed from a remote source. The radiation treatment can also be administered as internal therapy or brachytherapy wherein a radioactive source is placed inside the body close to cancer cells or a tumor mass. Also encompassed is the use of photodynamic therapy comprising the administration of photosensitizers, such as hematoporphyrin and its derivatives, Vertoporfin (BPD-MA), phthalocyanine, photosensitizer Pc4, demethoxy-hypocrellin A; and 2BA-2-DMHA.

In various embodiments, immunotherapy may comprise, for example, use of cancer vaccines and/or sensitized antigen presenting cells. In some embodiments, therapies include targeting cells in the tumor microenvironment or targeting immune cells. The immunotherapy can involve passive immunity for short-term protection of a host, achieved by the administration of pre-formed antibody directed against a cancer antigen or disease antigen (e.g., administration of a monoclonal antibody, optionally linked to a chemotherapeutic agent or toxin, to a tumor antigen). Immunotherapy can also focus on using the cytotoxic lymphocyte-recognized epitopes of cancer cell lines.

In various embodiments, hormonal therapy can include, for example, hormonal agonists, hormonal antagonists (e.g., flutamide, bicalutamide, tamoxifen, raloxifene, leuprolide acetate (LUPRON), LH-RH antagonists), inhibitors of hormone biosynthesis and processing, and steroids (e.g., dexamethasone, retinoids, deltoids, betamethasone, cortisol, cortisone, prednisone, dehydrotestosterone, glucocorticoids, mineralocorticoids, estrogen, testosterone, progestins), vitamin A derivatives (e.g., all-trans retinoic acid (ATRA)); vitamin D3 analogs; antigestagens (e.g., mifepristone, onapristone), or antiandrogens (e.g., cyproterone acetate).

Some embodiments of the present invention can be defined as any of the following numbered paragraphs:

1. A nanoparticle, comprising:
a core, wherein the core comprises at least one iron oxide;
a shell surrounding the core, wherein the shell comprises at least one polymer; and
at least one targeting moiety attached to the shell.
2. The nanoparticle of paragraph 1, wherein the at least one iron oxide is selected from the group consisting of FeO, Fe₂O₃, and combinations thereof.
3. The nanoparticle of paragraph 1, wherein the at least one polymer is at least one biocompatible polymer.
4. The nanoparticle of paragraph 1, wherein the at least one polymer is at least one polysaccharide.
5. The nanoparticle of paragraph 1, wherein the at least one polymer is selected from the group consisting of at least one dextran, at least one unfunctionalized dextran, at least one functionalized dextran, at least one unsubstituted dextran, at least one substituted dextran, and combinations thereof.
6. The nanoparticle of paragraph 1, wherein the at least one polymer is selected from the group consisting of carboxymethyl dextran, at least one dextran, and combinations thereof.
7. The nanoparticle of paragraph 5 or paragraph 6, wherein the at least one dextran is selected from the group consisting of a class 1 dextran, a class 2 dextran, a class 3 dextran, and combinations thereof.
8. The nanoparticle of paragraph 1, wherein the at least one targeting moiety is selected from heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof.
9. The nanoparticle of paragraph 1, further comprising at least one drug.
10. The nanoparticle of paragraph 9, wherein the drug is encapsulated in the nanoparticle.
11. The nanoparticle of paragraph 9, wherein the at least one drug is not a boron cluster.
12. The nanoparticle of paragraph 9, wherein the at least one drug is a therapeutic agent.
13. The nanoparticle of paragraph 9, wherein the at least one drug is selected from the group consisting of docetaxel (DXT), paclitaxel (PXT), bortezomib (Bort), cobozentanib (cabo), brefeldin A (BFA), and combinations thereof.
14. The nanoparticle of paragraph 1, further comprising at least one fluorescent dye.
15. The nanoparticle of paragraph 14, wherein the at least one fluorescent dye is encapsulated in the nanoparticle.
16. The nanoparticle of paragraph 14 or 15, wherein the nanoparticle is selected from angiopep-FH(DiR) and angiopep-FH(HMC).
17. The nanoparticle of paragraph 14, wherein the at least one fluorescent dye is a near infrared fluorescent dye.
18. The nanoparticle of paragraph 14, wherein the at least one fluorescent dye is selected from the group consisting of DiI, DiR, heptamethine cyanine (HMC), IR820, and combinations thereof.
19. The nanoparticle of paragraph 14, wherein the nanoparticle is a multimodal nanoparticle.
20. The nanoparticle of paragraph 9, further comprising at least one fluorescent dye.
21. The nanoparticle of paragraph 20, wherein the at least one fluorescent dye is encapsulated in the probe.
22. The nanoparticle of paragraph 20, wherein the at least one fluorescent dye is a near infrared fluorescent dye.
23. The nanoparticle of paragraph 20, wherein the at least one fluorescent dye is selected from the group consisting of DiI, DiR, heptamethine cyanine (HMC), IR820, and combinations thereof.
24. The nanoparticle of paragraph 20, wherein the nanoparticle is a multimodal nanoparticle.
25. A method for detecting and treating a cancer in a subject, comprising:
administering an effective amount of at least one nanoparticle of paragraph 9 or paragraph 20 to the subject, thereby contacting a tissue of the subject with the at least one nanoparticle such that the at least one nanoparticle binds to the tissue;
detecting the at least one nanoparticle bound to the tissue, wherein the presence of the at least one nanoparticle bound to the tissue is indicative of the cancer in the subject; and
delivering the at least one drug to the tissue thereby treating the cancer in the subject.
26. A method for detecting a cancer in a subject, comprising:
administering an effective amount of at least one nanoparticle of paragraph 1 or paragraph 20 to the subject, thereby contacting a tissue of the subject with the at least one nanoparticle such that the at least one nanoparticle binds to the tissue; and
detecting the at least one nanoparticle bound to the tissue, wherein the presence of the at least one nanoparticle bound to the tissue is indicative of the cancer in the subject.
27. The method of paragraph 26, further comprising administering a treatment to the subject.
28. A method for diagnosing and treating a cancer in a subject, comprising:
administering an effective amount of at least one nanoparticle of paragraph 9 or paragraph 20 to the subject, thereby contacting a tissue of the subject with the at least one nanoparticle such that the at least one nanoparticle binds to the tissue;
detecting the at least one nanoparticle bound to the tissue, wherein the presence of the at least one nanoparticle bound to the tissue is indicative of the cancer in the subject; and
delivering the at least one drug to the tissue thereby treating the cancer in the subject.
29. A method for diagnosing a cancer in a subject, comprising:
administering an effective amount of at least one nanoparticle of paragraph 1 or paragraph 20 to the subject, thereby contacting a tissue of the subject with the at least one nanoparticle such that the at least one nanoparticle binds to the tissue; and
detecting the at least one nanoparticle bound to the tissue, wherein the presence of the at least one nanoparticle bound to the tissue is indicative of the cancer in the subject.
30. The method of claim 29, further comprising administering a treatment to the subject.
31. A method for treating a cancer in a subject, comprising:
administering an effective amount of at least one nanoparticle of paragraph 9 or paragraph 20 to the subject, thereby contacting a tissue of the subject with the at least one nanoparticle such that the at least one nanoparticle binds to the tissue;
detecting the at least one nanoparticle bound to the tissue, wherein the presence of the at least one nanoparticle bound to the tissue is indicative of the cancer in the subject; and
delivering the at least one drug to the tissue thereby treating the cancer in the subject.
32. The method of any one of paragraphs 25, 26, 28, 29, or 31, wherein the at least one nanoparticle is detected using magnetic resonance imaging.
33. The method of claim of any one of paragraphs 25, 26, 28, 29, or 31, wherein the at least one nanoparticle is detected using fluorescence imaging.
34. The method of paragraph 33, wherein the fluorescence imaging is selected from the group consisting of near infrared fluorescence imaging, intraoperative fluorescence imaging, and combinations thereof.
35. The method of any one of paragraphs 25, 26, 28, 29, or 31, wherein the at least one nanoparticle is detected using magnetic resonance imaging and fluorescence imaging.
36. The method of paragraph 35, wherein the fluorescence imaging is selected from the group consisting of near infrared fluorescence imaging, intraoperative fluorescence imaging, and combinations thereof.
37. The method of any one of paragraphs 25, 26, 28, 29, or 31, wherein the cancer is selected from the group consisting of lung cancer, breast cancer, ovarian cancer, pancreatic cancer, head cancer, neck cancer, skin cancer, prostate cancer, brain cancer, and combinations thereof.
38. The method of paragraph 25, 26, 28, 29, or 31, wherein the cancer is metastasized.
39. The method of any one of paragraphs 25, 26, 28, 29, or 31, wherein the tissue is selected from the group consisting of cancerous tissue, cancer tissue, tumor, tumor tissue, and combinations thereof.
40. The method of any one of paragraphs 25, 28, or 31, further comprising administering at least one additional therapy to the subject.
41. The method of paragraph 35, wherein the additional therapy is selected from the group consisting of pharmacological therapy, biological therapy, cell therapy, gene therapy, chemotherapy, radiation therapy, hormonal therapy, surgery, immunotherapy, and combinations thereof.
42. A probe comprising at least one coated iron oxide nanoparticle; and at least one targeting moiety.
43. The probe of paragraph 42, wherein the at least one targeting moiety is attached to the at least one coated iron oxide nanoparticle.
44. The probe of paragraph 42, wherein the at least one coated iron oxide nanoparticle is selected from the group consisting of Ferumoxytol, Ferumoxides, Ferucarbotran, Ferumoxtran-10, NC100150, VSOP C184, and combinations thereof.
45. The probe of paragraph 42, wherein the at least one targeting moiety is selected from heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof.
46. The probe of paragraph 42, further comprising at least one drug.
47. The probe of paragraph 46, wherein the at least one drug is encapsulated in the probe.
48. The probe of paragraph 46, wherein the at least one drug is not a boron cluster.
49. The probe of paragraph 46, wherein the at least one drug is a therapeutic agent.
50. The probe of paragraph 46, wherein the at least one drug is selected from the group consisting of docetaxel (DXT), paclitaxel (PXT), bortezomib (Bort), cobozentanib (cabo), brefeldin A (BFA), and combinations thereof.
51. The probe of paragraph 42, further comprising at least one fluorescent dye.
52. The probe of paragraph 51, wherein the at least one fluorescent dye is encapsulated in the probe.
53. The probe of paragraph 42 or paragraph 52, wherein the probe is selected from angiopep-FH(DiR) and angiopep-FH(HMC).
54. The probe of paragraph 51, wherein the at least one fluorescent dye is a near infrared fluorescent dye.
55. The probe of paragraph 51, wherein the at least one fluorescent dye is selected from the group consisting of DiI, DiR, heptamethine cyanine (HMC), IR820, and combinations thereof.
56. The probe of paragraph 41 or 51, wherein the probe is a multimodal probe.
57. The probe of paragraph 46, further comprising at least one fluorescent dye.
58. The probe of paragraph 57, wherein the at least one fluorescent dye is encapsulated in the probe.
59. The probe of paragraph 57, wherein the at least one fluorescent dye is a near infrared fluorescent dye.
60. The probe of paragraph 57, wherein the at least one fluorescent dye is selected from the group consisting of DiI, DiR, heptamethine cyanine (HMC), IR820, and combinations thereof
61. The probe of paragraph 57, wherein the probe is a multimodal probe.
62. A method for detecting and treating a cancer in a subject, comprising:
administering an effective amount of at least one probe of paragraph 46 or paragraph 57 to the subject, thereby contacting a tissue of the subject with the at least one probe such that the at least one probe binds to the tissue;
detecting the at least one probe bound to the tissue, wherein the presence of the at least one probe bound to the tissue is indicative of the cancer in the subject; and
delivering the at least one drug to the tissue thereby treating the cancer in the subject.
63. A method for detecting a cancer in a subject, comprising:
administering an effective amount of at least one probe of paragraph 42 or paragraph 51 to the subject, thereby contacting a tissue of the subject with the at least one probe such that the at least one probe binds to the tissue; and
detecting the at least one probe bound to the tissue, wherein the presence of the at least one probe bound to the tissue is indicative of the cancer in the subject.
64. The method of paragraph 63, further comprising administering a treatment to the subject.
65. A method for diagnosing and treating a cancer in a subject, comprising:
administering an effective amount of at least one probe of paragraph 46 or paragraph 57 to the subject, thereby contacting a tissue of the subject with the at least one probe such that the at least one probe binds to the tissue;
detecting the at least one probe bound to the tissue, wherein the presence of the at least one probe bound to the tissue is indicative of the cancer in the subject; and
delivering the at least one drug to the tissue thereby treating the cancer in the subject.
66. A method for diagnosing a cancer in a subject, comprising:
administering an effective amount of at least one probe of paragraph 42 or paragraph 51 to the subject, thereby contacting a tissue of the subject with the at least one probe such that the at least one probe binds to the tissue; and
detecting the at least one probe bound to the tissue, wherein the presence of the at least one probe bound to the tissue is indicative of the cancer in the subject.
67. The method of paragraph 66, further comprising administering a treatment to the subject.
68. A method for treating a cancer in a subject, comprising:
administering an effective amount of at least one probe of paragraph 46 or paragraph 57 to the subject, thereby contacting a tissue of the subject with the at least one probe such that the at least one probe binds to the tissue;
detecting the at least one probe bound to the tissue, wherein the presence of the at least one probe bound to the tissue is indicative of the cancer in the subject; and
delivering the drug to the tissue thereby treating the cancer in the subject.
69. The method of any one of paragraphs 62, 63, 65, 66, or 68, wherein the at least one probe is detected using magnetic resonance imaging.
70. The method of any one of paragraphs 62, 63, 65, 66, or 68, wherein the at least one probe is detected using fluorescence imaging.
71. The method of paragraph 70, wherein the fluorescence imaging is selected from the group consisting of near infrared fluorescence imaging, intraoperative fluorescence imaging, and combinations thereof.
72. The method of any one of paragraphs 62, 63, 65, 66, or 68, wherein the at least one probe is detected using magnetic resonance imaging and fluorescence imaging.
73. The method of paragraph 72, wherein the fluorescence imaging is selected from the group consisting of near infrared fluorescence imaging, intraoperative fluorescence imaging, and combinations thereof.
74. The method of any one of paragraphs 62, 63, 65, 66, or 68, wherein the cancer is selected from the group consisting of lung cancer, breast cancer, ovarian cancer, pancreatic cancer, head cancer, neck cancer, skin cancer, prostate cancer, brain cancer, and combinations thereof.
75. The method of any one of paragraphs 62, 63, 65, 66, or 68, wherein the cancer is metastasized.
76. The method of any one of paragraphs 62, 63, 65, 66, or 68, wherein the tissue is selected from the group consisting of cancerous tissue, cancer tissue, tumor, tumor tissue, and combinations thereof.
77. The method of any one of paragraphs 62, 65, or 68, further comprising administering at least one additional therapy to the subject.
78. The method of paragraph 77, wherein the additional therapy is selected from the group consisting of pharmacological therapy, biological therapy, cell therapy, gene therapy, chemotherapy, radiation therapy, hormonal therapy, surgery, immunotherapy, and combinations thereof.
79. A pharmaceutical composition comprising at least one nanoparticle of any one of paragraphs 1 to 24.
80. A pharmaceutical composition comprising at least one probe of any one of paragraphs 42 to 61.
81. The nanoparticle of paragraph 1, wherein the nanoparticle does not comprise a boron cluster.
82. The probe of paragraph 42, wherein the probe does not comprise a boron cluster.
83. The nanoparticle of paragraph 1, wherein the nanoparticle does not comprise a compound comprising boron.
84. The probe of paragraph 42, wherein the probe does not comprise a compound comprising boron.

Some embodiments of the present invention can be defined as any of the following numbered paragraphs:

85. A nanoparticle, comprising:
a core, wherein the core comprises at least one iron oxide;
a shell surrounding the core, wherein the shell comprises at least one polymer; and
at least one targeting moiety attached to the shell,
   wherein the nanoparticle does not comprise boron.
      86. The nanoparticle of paragraph 85, wherein the at least one iron oxide is selected from the group consisting of FeO, Fe₂O₃, and combinations thereof.
      87. The nanoparticle of paragraph 85, wherein the at least one polymer is at least one biocompatible polymer.
      88. The nanoparticle of paragraph 85, wherein the at least one polymer is at least one polysaccharide.
      89. The nanoparticle of paragraph 85, wherein the at least one polymer is selected from the group consisting of at least one dextran, at least one unfunctionalized dextran, at least one functionalized dextran, at least one unsubstituted dextran, at least one substituted dextran, and combinations thereof.
      90. The nanoparticle of paragraph 85, wherein the at least one polymer is selected from the group consisting of carboxymethyl dextran, at least one dextran, and combinations thereof.
      91. The nanoparticle of paragraph 89 or paragraph 90, wherein the at least one dextran is selected from the group consisting of a class 1 dextran, a class 2 dextran, a class 3 dextran, and combinations thereof.
      92. The nanoparticle of paragraph 85, wherein the at least one targeting moiety is selected from heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof.
      93. The nanoparticle of paragraph 85, further comprising at least one drug.
      94. The nanoparticle of paragraph 93, wherein the at least one drug is selected from the group consisting of docetaxel (DXT), paclitaxel (PXT), bortezomib (Bort), cobozentanib (cabo), brefeldin A (BFA), and combinations thereof.
      95. The nanoparticle of paragraph 85, further comprising at least one fluorescent dye.
      95. The nanoparticle of paragraph 95, wherein the at least one fluorescent dye is a near infrared fluorescent dye.
      97. The nanoparticle of paragraph 95, wherein the at least one fluorescent dye is selected from the group consisting of DiI, DiR, heptamethine cyanine (HMC), IR820, and combinations thereof.
      98. The nanoparticle of paragraph 93, further comprising at least one fluorescent dye.
      99. The nanoparticle of paragraph 98, wherein the at least one fluorescent dye is a near infrared fluorescent dye.
      100. The nanoparticle of paragraph 98, wherein the at least one fluorescent dye is selected from the group consisting of DiI, DiR, heptamethine cyanine (HMC), IR820, and combinations thereof.
      101. A method for detecting and treating a cancer in a subject, comprising:
administering an effective amount of at least one nanoparticle of paragraph 93 or paragraph 98 to the subject, thereby contacting a tissue of the subject with the at least one nanoparticle such that the at least one nanoparticle binds to the tissue;
detecting the at least one nanoparticle bound to the tissue, wherein the presence of the at least one nanoparticle bound to the tissue is indicative of the cancer in the subject; and
delivering the at least one drug to the tissue thereby treating the cancer in the subject.
   102. A method for detecting a cancer in a subject, comprising:
administering an effective amount of at least one nanoparticle of paragraph 85 or paragraph 98 to the subject, thereby contacting a tissue of the subject with the at least one nanoparticle such that the at least one nanoparticle binds to the tissue; and
detecting the at least one nanoparticle bound to the tissue, wherein the presence of the at least one nanoparticle bound to the tissue is indicative of the cancer in the subject.
   103. The method of paragraph 102, further comprising administering a treatment to the subject.
   104. The method of paragraph 101 or paragraph 102, wherein the nanoparticle is detected by an imaging method.
   105. The method of paragraph 104, wherein the imaging method is selected from the group consisting of magnetic resonance imaging, fluorescence imaging, and combinations thereof.
   106. The method of paragraph 101 or paragraph 102, wherein the cancer is selected from the group consisting of lung cancer, breast cancer, ovarian cancer, pancreatic cancer, head cancer, neck cancer, skin cancer, prostate cancer, brain cancer, and combinations thereof.
   107. The method of paragraph 101 or paragraph 102, wherein the cancer is metastasized.
   108. The method of paragraph 101 or paragraph 102, wherein the tissue is selected from the group consisting of cancerous tissue, cancer tissue, tumor, tumor tissue, and combinations thereof.
   109. The method of paragraph 101, further comprising administering at least one additional therapy to the subject.
   110. The method of paragraph 109, wherein the additional therapy is selected from the group consisting of pharmacological therapy, biological therapy, cell therapy, gene therapy, chemotherapy, radiation therapy, hormonal therapy, surgery, immunotherapy, and combinations thereof.
   111. The method of paragraph 103, wherein the treatment is a cancer treatment.
   112. A probe comprising at least one coated iron oxide nanoparticle; and at least one targeting moiety, wherein the probe does not comprise boron.
   113. The probe of paragraph 112, wherein the at least one coated iron oxide nanoparticle is selected from the group consisting of Ferumoxytol, Ferumoxides, Ferucarbotran, Ferumoxtran-10, NC100150, VSOP C184, and combinations thereof.
   114. The probe of paragraph 112, wherein the at least one targeting moiety is selected from heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof.
   115. The probe of paragraph 112, further comprising at least one drug.
   116. The probe of paragraph 115, wherein the at least one drug is selected from the group consisting of docetaxel (DXT), paclitaxel (PXT), bortezomib (Bort), cobozentanib (cabo), brefeldin A (BFA), and combinations thereof.
   117. The probe of paragraph 115, further comprising at least one fluorescent dye.
   118. The nanoparticle of paragraph 85, wherein the at least one targeting moiety is selected from an antibody that selectively targets cancer cells, a peptide that selectively targets cancer cells, and combinations thereof.

### EXAMPLES

The following examples are not intended to limit the scope of the claims to the invention, but are rather intended to be exemplary of certain embodiments. Any variations in the exemplified methods which occur to the skilled artisan are intended to fall within the scope of the present invention. The invention is further illustrated by the following examples which are intended to be purely exemplary of the invention, and which should not be construed as limiting the invention in any way. The following examples are illustrative only, and are not intended to limit, in any manner, any of the aspects described herein. The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

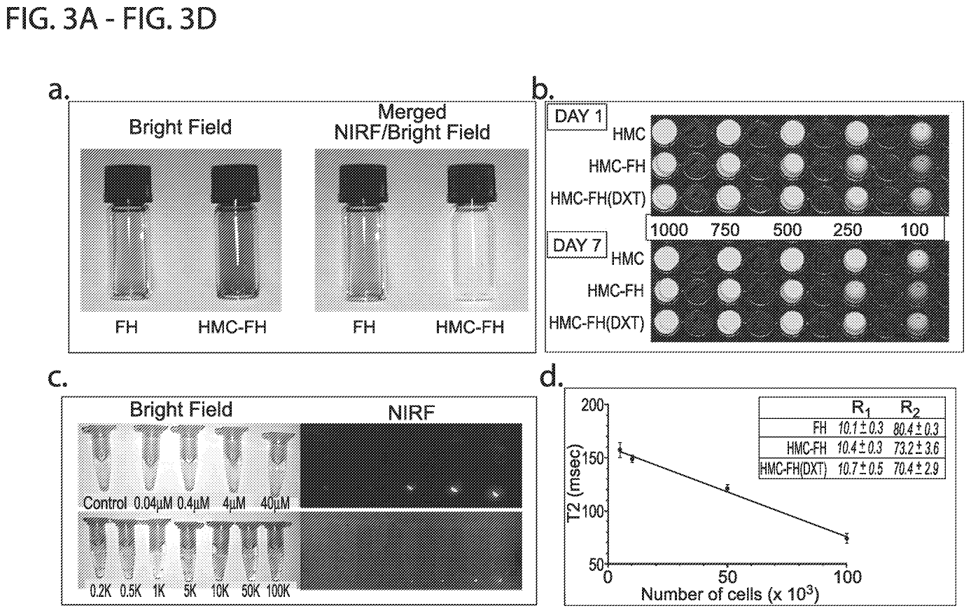

Multimodal HMC-FH nanoconjugates are sensitive dual near infrared and magnetic probes. Considering its exquisite tumor affinity and desirable NIRF properties, HMC was conjugated to FH for the fluorescent intraoperative detection of prostate cancer tumor. To achieve this, we have initially modified HMC with a lysine linker to yield HMC-Lys (FIG. 2); that is then conjugated onto the carboxylic acid groups in FH's carboxymethyl dextran coating. HMC conjugation does not affect the size, polydispersity and stability of the nanoparticles in aqueous buffers. Furthermore, the fluorescent properties of HMC are not affected upon conjugation with FH as no quenching was observed (FIG. 3A-FIG. 3D). Upon excitation at 785 nm, bright and stable NIR fluorescence is observed with a limit of detection in the low nM range in HMC, HMC-FH and HMC-FH(DXT) samples (FIG. 3B). The intense NIRF emission of HMC-FH in combination with the sensitive NIFR signal detection allowed for the use of down to 0.4 uM (400 nm) of HMC-FH to detect down to 5,000 cells (FIG. 3C). Furthermore, the magnetic relaxation properties of the HMC-FH nanoparticles are not significantly affected by HMC conjugation or encapsulation of DXT (FIG. 3D), facilitating the detection by MRI of down to 50,000 cells in vitro. These results indicate that neither the MR fluorescence properties of HMC nor the magnetic relaxation properties of FH are affected by the conjugation of HMC onto FH and that the resulting HMC-FH is a sensitive multimodal probe for the detection of cancer cells by MRI and NIRF.

### Example 2

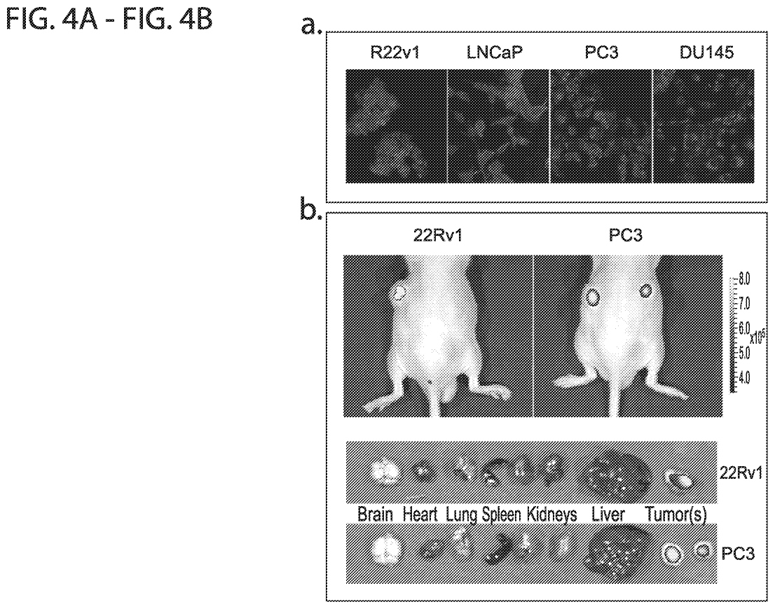

HMC-FH targets and fluorescently label PCa in culture cells and tumors in vivo. Next we tested the ability of HMC-FH to target and internalize into prostate cancer cell line. For these studies, two androgen-sensitive (22Rv1/LNCaP) and two androgen-independent (PC3/DU145) cell lines were selected. Results show bright MR fluorescence in the cytoplasm of all the cells studied indicating successful internalization of the HMC-FH (40004) within 72 h (FIG. 4A). When mice with 22Rv1 and PC3 subcutaneous xerographs were injected with HMC-FH (1 mg HMC and 4 mgFe/kg mice) and imaged with the Perkin Elmer's In Vivo Imaging System (IVIS), fluorescent was localized to the tumors with minimal fluorescent in the other organs (FIG. 4B). These in vivo results are similar to those obtained with the HMC dye along and show that the cancer targeting ability of HMC is not compromised in HMC-FH.

### Example 3

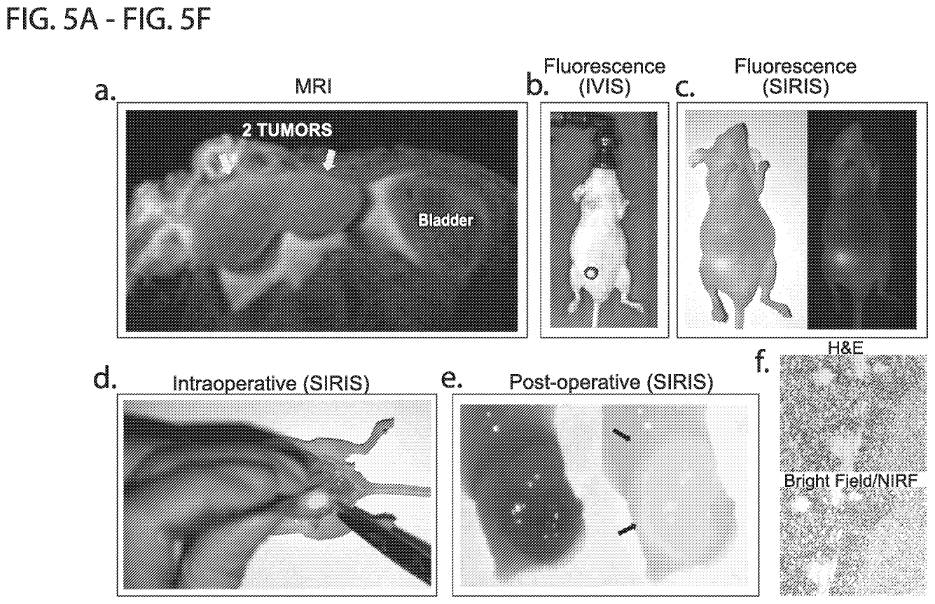

HMC-FH can assist in the intraoperative detection of PCa tumors. To demonstrate the feasibility of HMC-FH to specifically visualize prostate tumors, we generated an orthotopic mouse prostate xenograph by injecting 1×10⁶ cells directly in the right lobe of the mouse prostate. After 2 weeks to allow tumor formation, an MR pre-surgical image indicates the presence of two tumor grafts on the mouse prostate right lobe (FIG. 5A). Then, HMC-FH (1 mg HMC and 4 mgFe/kg mice) was injected i.v. and imaged 72 hours after injection to allow for elimination of non-tumor associated, long circulating HMC-FH. This step was necessary, even though tumor associated fluorescence was observed within 24 H, to reduce the background fluorescence of circulating HMC-FH. Results clearly show a bright fluorescent spot near the prostate right lobe area on the living mouse (FIG. 5B, FIG. 5C). Upon intraoperative assessment to expose the abdominal area where the mouse prostate is located, two adjacent fluorescent tumors were seen on the prostate right lobe, clearly delineating tumor margins and facilitating surgical extraction (FIG. 5D). Post-operative visualization of the extracted fluorescent tissue shows the presence of the tumors (FIG. 5E), while histopathology examination show that the fluorescent nanoparticle specifically target the cancer tissue with no accumulation in the adjacent normal tissue (FIG. 5F). Taken together, these preliminary studies show the feasibility HMC-FH to fluorescently label prostate cancer tumors, identifying tumor margins and facilitating their surgical extraction.

### Example 4

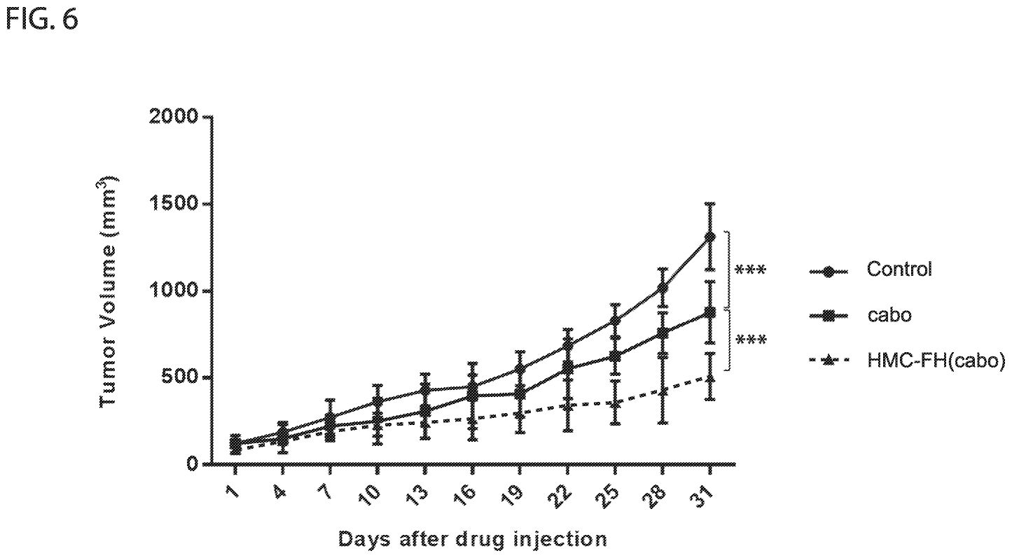

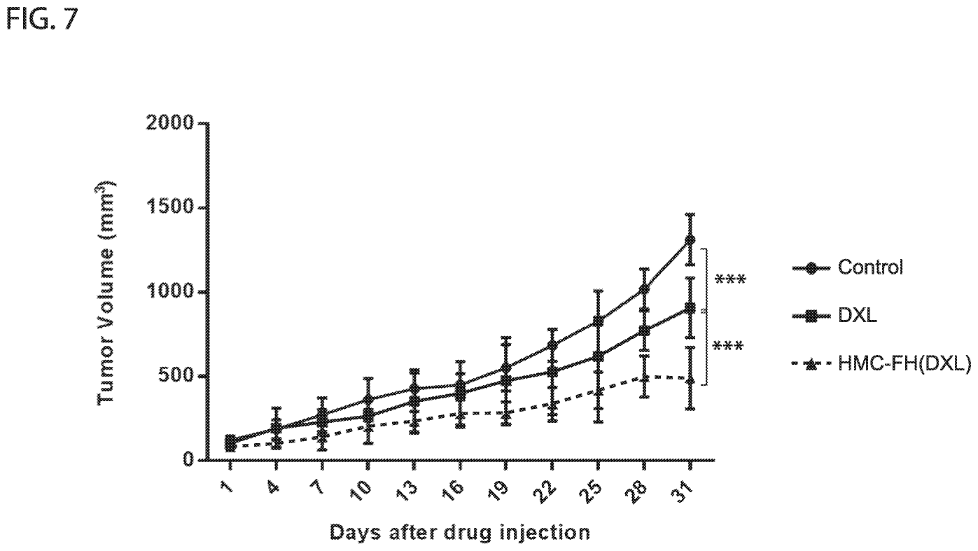

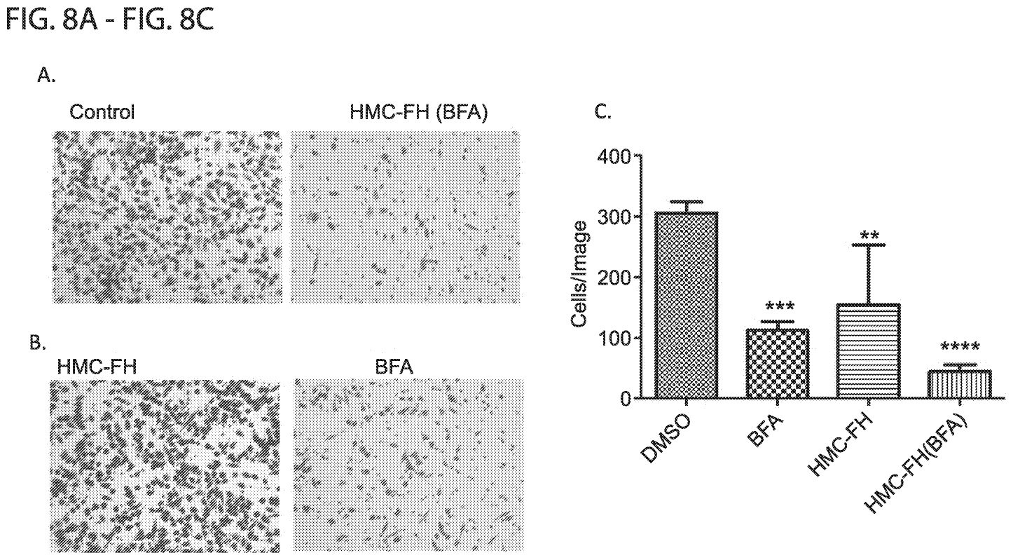

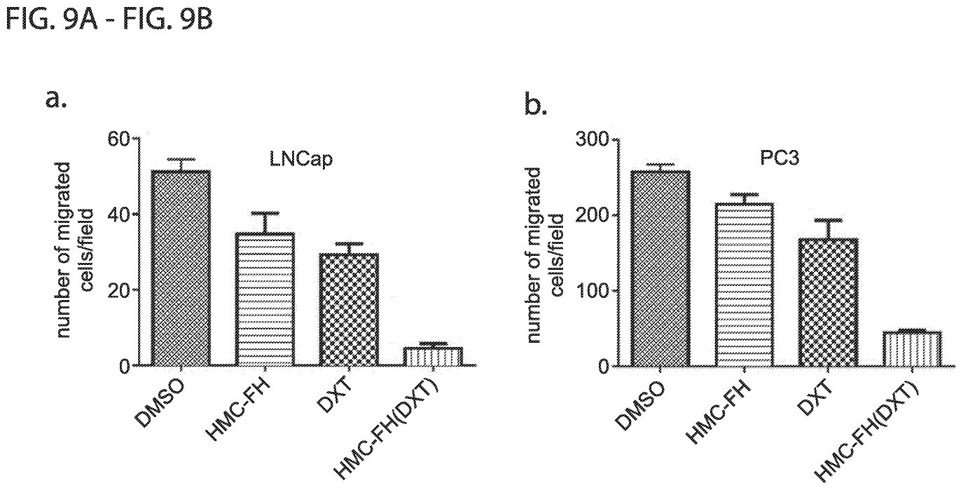

HMC-FH(Drug) image-guided DXT delivery to PCA tumors. In these experiments, we conjugated various drugs into the HMC-conjugated Feraheme to create an HMC-FH(Drug) agent. As initial drugs to treat prostate cancer, we have used docetaxel (DXT), cabozentanib (cabo), and brefeldin A (BFA) to encapsulate into HMC-FH. We then injected the HMC-FH(DXT) or HMC-FH(cabo) (1 mg HMC, 3 mg DXT (or Cabo) and 4 mgFe/kg mice) to mice bearing subcutaneous 22Rv1 xenographs and measured tumor size for a period of 31 days. Results showed that both HMC-FH(cabo) (FIG. 6) or HMC-FH(DXT) (FIG. 7) were more efficient that the drug along in reducing the growth and size of the tumors, at equivalent DXT and FH amounts. These results suggest that the encapsulation of drugs unto HMC-FH improve the efficacy the drug in killing 22Rv1 prostate cancer tumors while also allowing image-guided assessment of drug delivery. We also performed cell migration studies of prostate cancer cells incubated with HMC-FH(BFA) (FIG. 8) or HMC-FH(DXT) (FIG. 9A-FIG. 9B). Results show that the cell migration of prostate cancer cells treated with either preparations of (HMC-FH(BFA) or HMC-FH(DXT)) was dramatically reduced as opposed to control non-treated cells. Surprisingly, the observed reduction in migration in cell treated the HMC-FH(Drug) formulation was larger than the one observed with the drugs along. These results suggest that the studied HMC-FH(Drug) formulations inhibit cell migration and could potentially inhibit metastasis in vivo better than the drugs along.

### Example 5

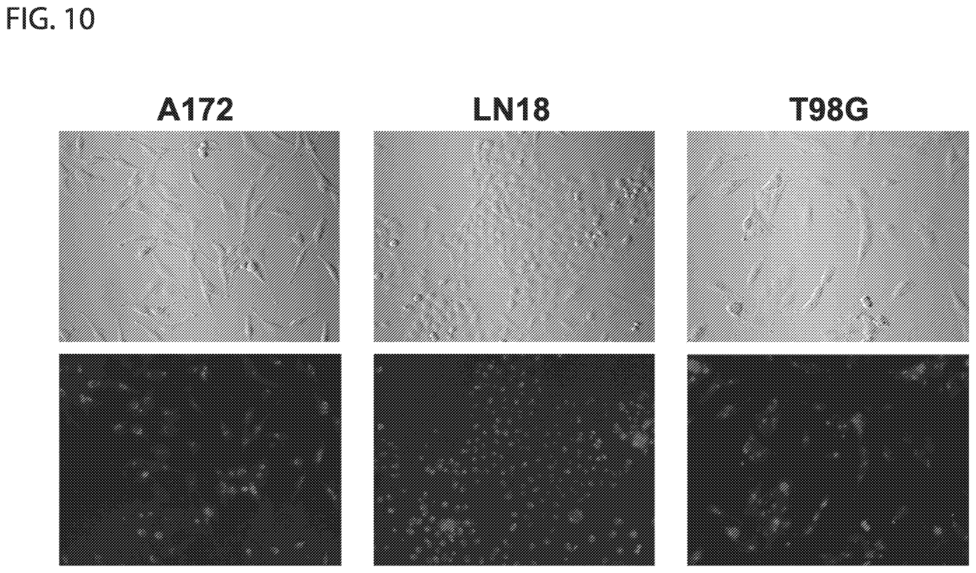

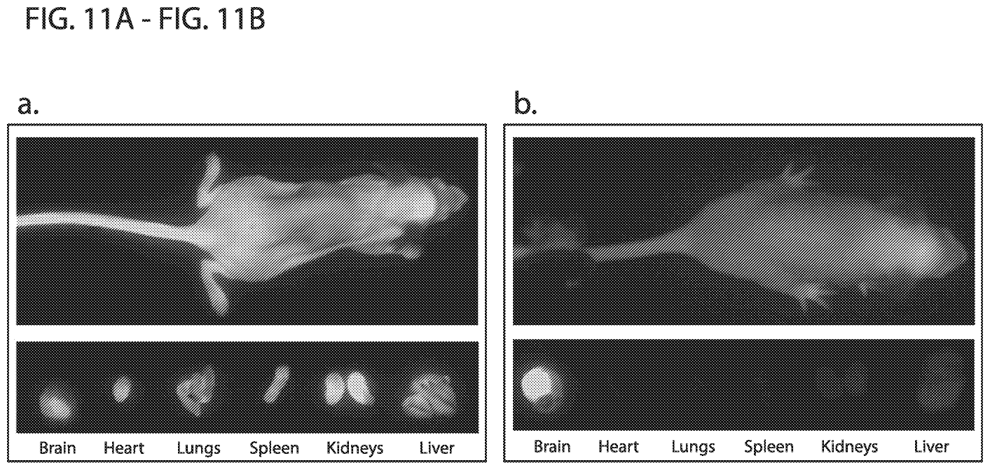

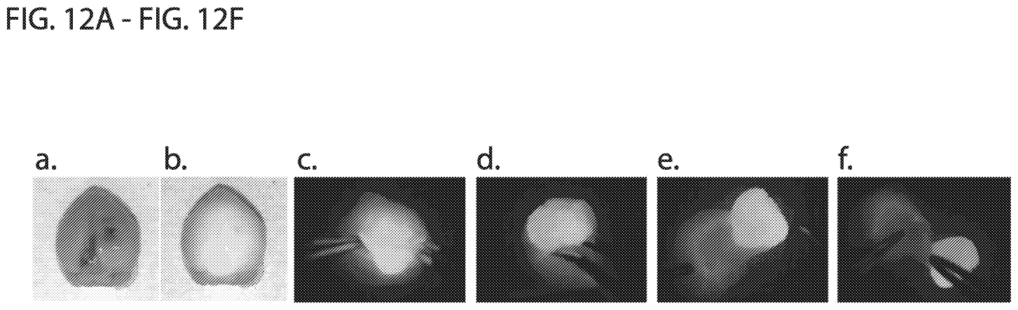

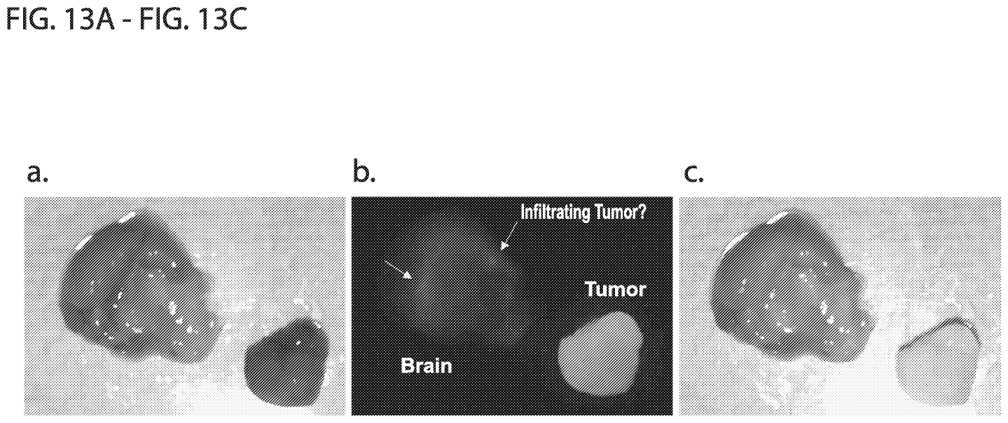

HMC-FH targets and fluorescently label glioblastoma (GBM) tumors in vivo. The ability of HMC-FH to target, accumulate and retain within intracranial U87 GBM tumors in mice was tested. In these experiments, HMC-FH was injected i.v. by tail vein injection and allowed to circulate for 24 H in a live mouse. Next day (24 H) the mouse was imaged with a near infrared camera. Fluorescence was detected throughout the whole mouse, suggesting that the nanoparticles are still in circulation after 24 H (FIG. 10). After mouse euthanasia, its vital organs were taken out and imaged with the near infrared camera. Intense fluorescence was observed in each of the organs, including the brain tumor. In another experiment, the mouse was injected with HMC-FH but in this case whole body fluorescence was imaged 7 days after injection of HMC-FH. Within 7 days, fluorescence was not observed in all major organs, however a very intense fluorescence was observed in the mouse GBM tumor, indicating accumulation of the HMC-FH nanoparticles within the GBM tumor (FIG. 11A-FIG. 11B). Furthermore, accumulation of the HMC-FH can be clearly visualized by monitoring HMC near infrared fluorescence, allowing the detection of the GBM tumor margins. FIG. 12A-FIG. 12F shows snap shots from a movie of a mouse brain showing that the HMC-FH fluorescence facilitates identification of tumor margins and removal of the tumor from the brain. “Post-surgical” visualization of the tumor vs the brain clearly shows the bright fluorescence in the tumor mass, with minimal to no fluorescence in the brain (FIG. 13A-FIG. 13C). Taken together, results from these experiments (invention) indicate that the HMC-FH preferentially accumulates and it is retained for at least 7 days in GBM tumor tissue, clearly allowing visualization of tumor margins and complete extraction of the tumors. It may also identify leftover infiltrating tumor cells or tumor tissue left behind in the brain. Without the use of a near infrared method to monitor these infiltrating tumors, the surgery would not have been successful, and tumor recurrence would have had happened in a couple of mouths.

### Example 6

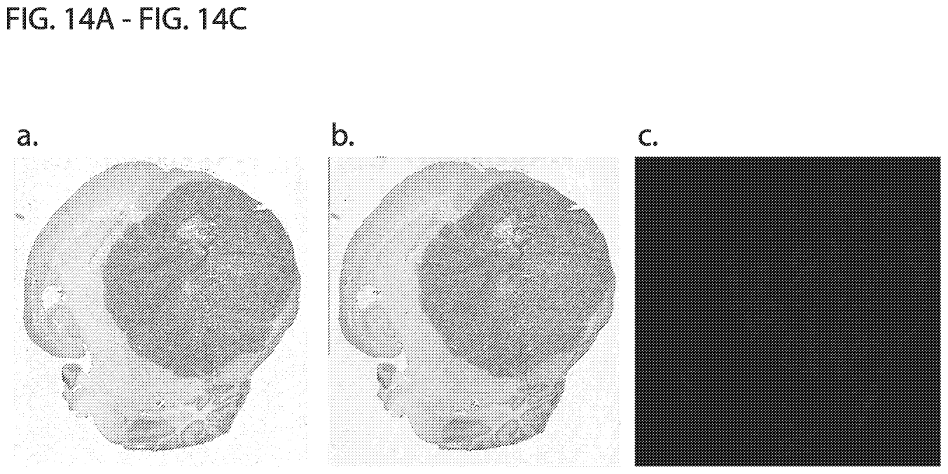

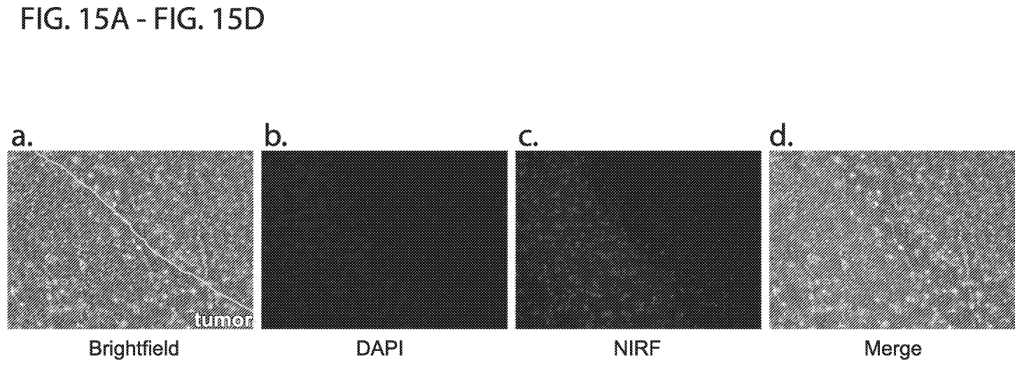

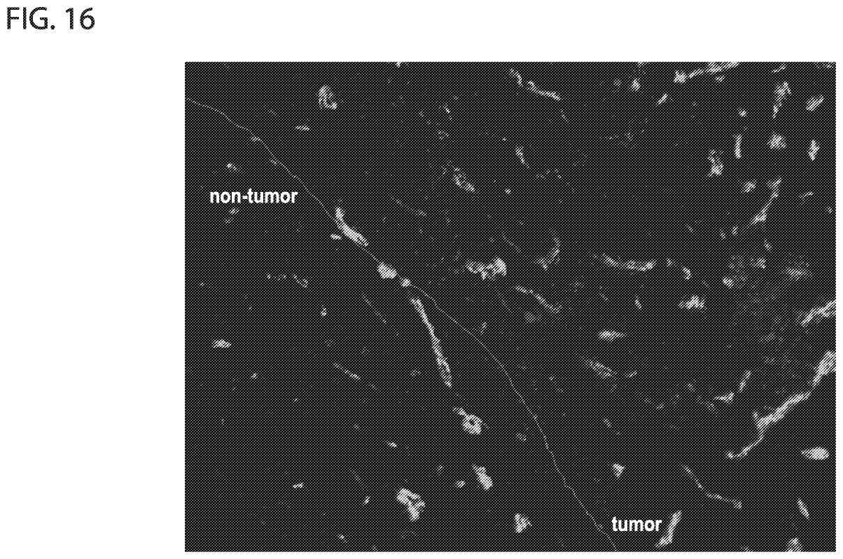

HMC-FH accumulates in brain tumors tissue, crossing the BBB. Next, we performed H&E staining and fluorescence imaging of mouse brain slides fixed in OCT. FIG. 14A-FIG. 14C show an image of a brain slide clearly indicating a brain tumor mass by bright field (FIG. 14A) and H&E (FIG. 14B). This tumor area matches the area identified by near infrared imaging (FIG. 14C). These results further demonstrate specific HMC-FH accumulation in tumor tissue. A higher magnification image near the tumor boundary shows a large accumulation at the cellular level of the HMC-FH, judged by the intense near infrared fluorescence in the tumor area (FIG. 15A-FIG. 15D). Further staining experiments of the brain slides with a von Willebrand factor (vWF) antibody, that mark vascular endothelial cells, indicates that the near infrared fluorescence (red) of the nanoparticles does not co-localize with the fluorescence of the vWF (green), suggesting that the HMC-FH is not associated or trapped in the vasculature and rather have crossed the BBB (FIG. 16).

### Example 7

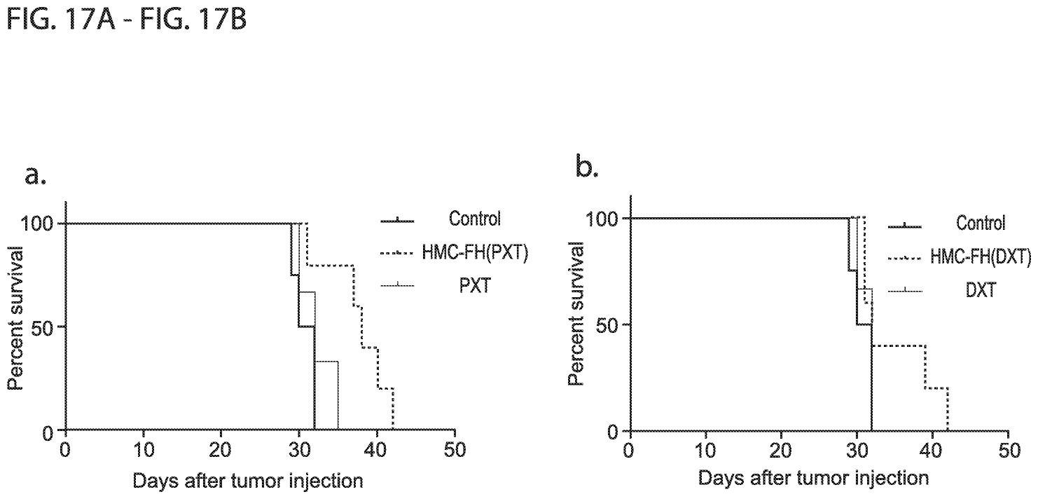

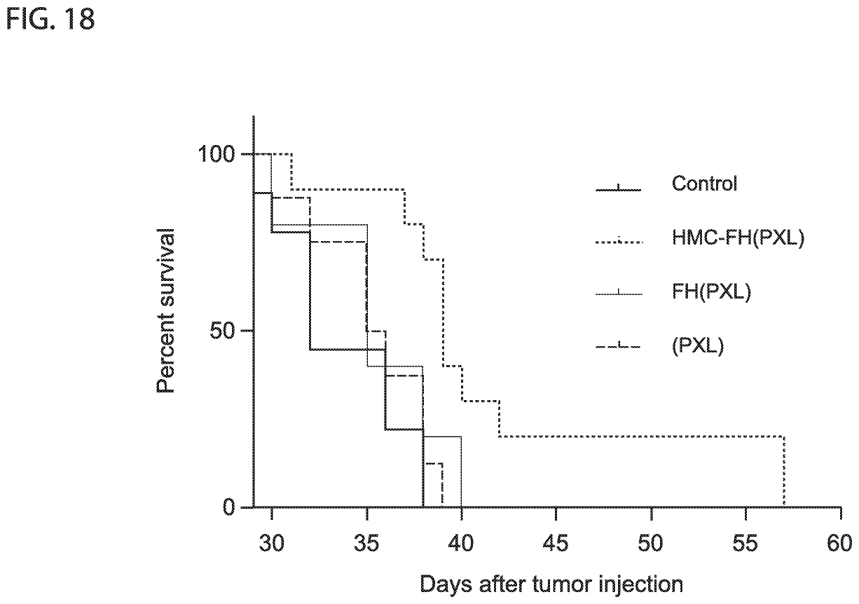

HMC-FH(PXL) and HMC-FH(BFA) increase the survival of mice with GBM tumors. In these experiments, we injected mice (n=5) with intracranial U87 GBM tumors with HMC-FH encapsulated with either paclitaxel (PTX) or docetaxel (DXT). As control, we injected either PBS or the corresponding drug along at equal concentrations. In initial studies, treatment started “late”, 14 days after tumor implantation. Survival studies in mice show that both the HMC-FH(PXL) and HMC-FH(DXT) performed better in enhancing mice survival than the drug along (FIG. 17A-FIG. 17B). HMC-FH(PXL) (FIG. 17A) performed better than HMC-FH(DXL) (FIG. 17B), while the drugs along performed similar to the mice treated with PBS (control), as these drugs do not cross the BBB. In additional studies, treatment was started earlier, 5 days after injection, to find out if an early treatment would improve survival. Impressive results were obtained by injecting HMC-FH(PXL), 5 days after tumor implantation. HMC-FH(PXL) outperformed not only the PXL along, but also FH(PXL), which does not contain HMC (FIG. 18). This indicates that HMC is essential in enhancing the survival of FH nanoparticles encapsulating a drug (PXL), due to the fact that HMC facilitates the crossing of the BBB (FIG. 16).

### Example 8

BFA and HMC-FH(BFA) increase the survival of mice with GBM tumors and decrease cell migration. Brefeldin A (BFA) is a small macrocylic lactone which inhibits protein transport between the endoplasmic reticulum (ER) and the Golgi^{[46-49]} This inhibition results in accumulation of proteins in the ER triggering activation of an unfolded protein response (UPR) and eventual ER stress, which results in cell death via apoptosis. In particular, BFA prevents the formation of transport vesicles that move proteins between the ER and Golgi by inhibition of ADP ribosylation factor (ARF1), a key regulator of vesicular formation and trafficking. This inhibition is believed to occur by the direct binding of BFA to an interface formed between ARF1 and guanine exchanged factors (GBF1, BIG1 or BIG2), which activate ARF1^{[50]}. In other words, BFA inhibits the activation of ARF1 by these guanine exchange factors. ARF1 has recently been found to be involved in an increasing number of cancers, including breast, ovarian, prostate, brain and pancreatic tumors, among others, where its upregulation plays a role in enhancing cell proliferation, invasiveness and progression as well as regulating epithelial-mesenchymal transition. In addition, ARF1 upregulation has also been found to be a predictor of poor clinical outcome in triple negative breast cancer^{[51]}. Taken together, these literature reports suggest that ARF is a key molecular target for cancer therapy and that BFA can be explored as a potential new therapeutic agent. BFA has cytotoxic effects on a variety of cancer cell lines. In addition, BFA reduces cell migration and cell adhesion by reducing the levels of MMP-9, MUC1 and integrin in cancer cells. However, despite its well-documented potential as a cancer therapeutic, and well-known mechanism of action, the clinical translation of BFA faces major limitations. Its low aqueous solubility, poor tumor uptake and biodistribution, hampers the development of clinical formulations. In contrast to Docetaxel and other taxanes that are hydrophobic and are administered using a non-aqueous vehicle containing Cremophor, similar formulations have not been developed for BFA. Therefore, there is a need to develop effective in vivo delivery methods for BFA. In these experiments, we use Feraheme, an FDA approved nanoparticle formulation to deliver BFA. Feraheme efficiently encapsulates and solubilizes BFA, and when the resulting FH(BFA) formulation is conjugated with HMC it efficiently delivers BFA to brain tumors.

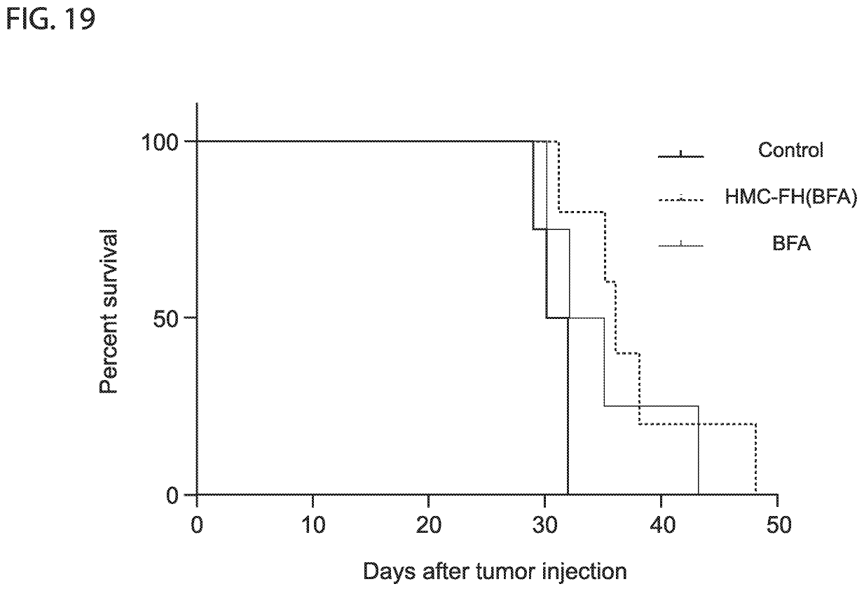

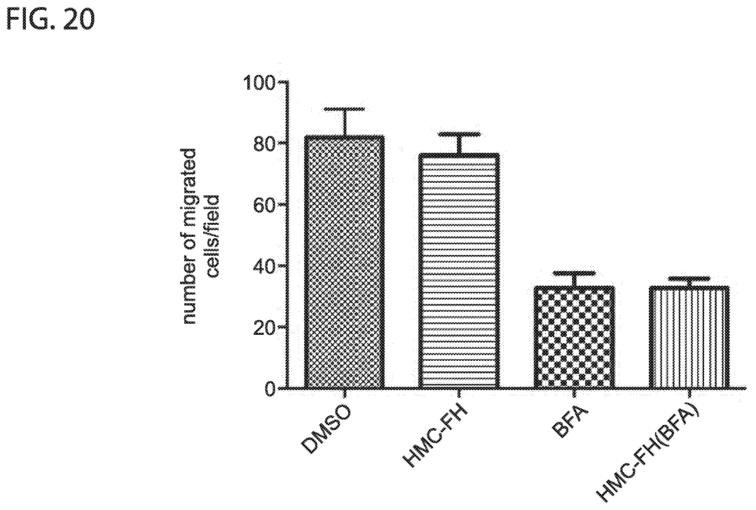

In initial animal studies using BFA and HMC-FH(BFA), we injected mice (n=5) with intracranial U87 GBM tumors with either BFA (dissolved in DMSO) or HMC-FH(BFA) in saline. In these studies, treatment started “late”, 14 days after tumor implantation. Survival studies in mice show that both BFA and HMC-FH(BFA) enhanced mice survival, with HMC-FH(BFA) performing slightly better (FIG. 19). The increase survival of BFA treated mice, contrast with results obtained with DXL and PXL treated mice (FIG. 17A-FIG. 17B, and FIG. 18), perhaps BFA crosses the BBB, while DXT and PXL does not. The ability of BFA to cross the BBB has not been studied or reported, to our knowledge. However, due to its poor solubility, it is advantageous to encapsulate BFA in HMC-FH. Finally, both BFA and HMC-FH(BFA) decrease cell migration in U87 cells, suggesting that these nanoparticles can prevent the migration and infiltration of GBM cells throughout the brain.

### Example 9

PSMA-Targeting-Feraheme nanoparticles. In this set of experiments, glutamate was conjugated to the carboxylic acid groups on Feraheme. The amine group (—NH₂) on the glutamate was conjugated with the carboxylic acid group (—COOH) on the carboxymethyldextran coated of Feraheme using EDC/HNS chemistry. This results in conjugation of multiple glutamate ligands to the surface of Feraheme. The resulting Glutamate-Feraheme (GLU-FH) nanoparticles are characterized by DLS (size), and zeta potential (charge). Both the Glutamate conjugate (GLU-FH) and the folate conjugate (FOL-FH) have been synthesized.

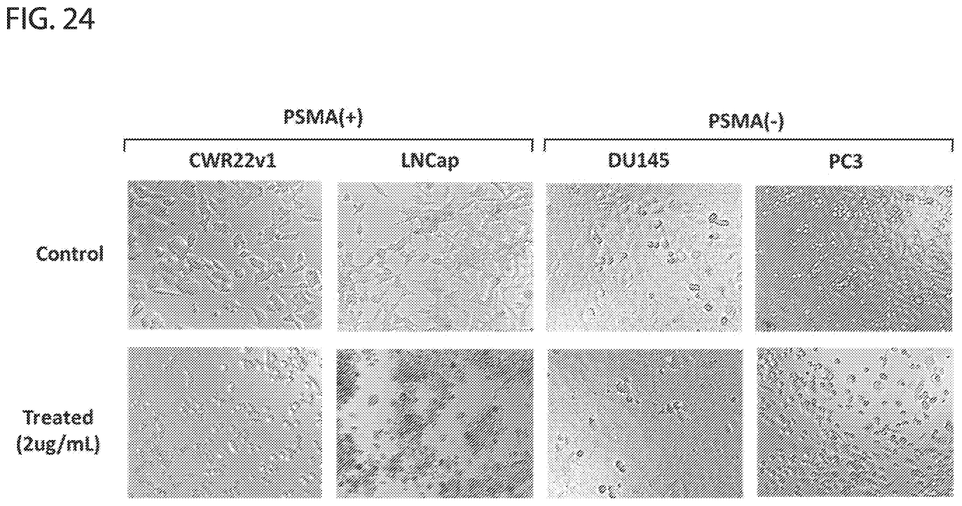

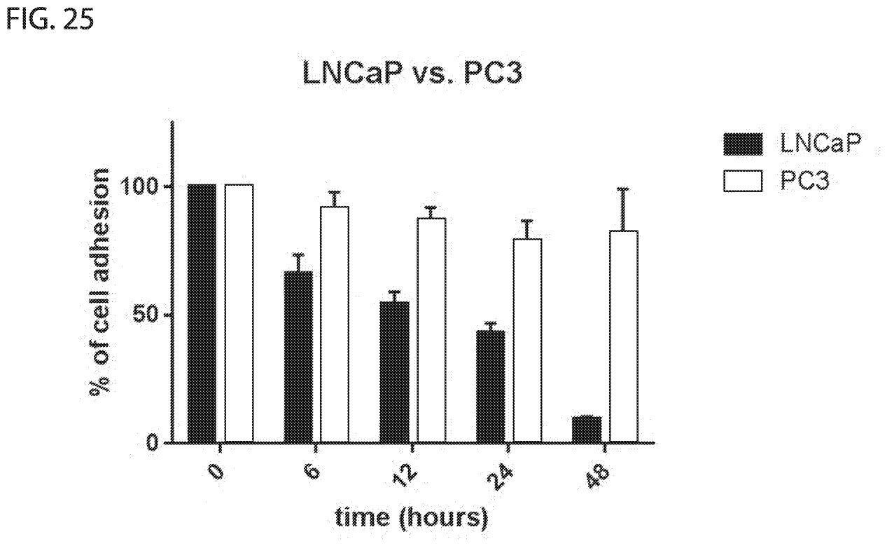

Theranostics PSMA-Targeting Feraheme (BF) nanoparticles. In this set of experiments, we have encapsulated BFA into the PSMA targeting Feraheme nanoparticles. Our studies using Glu-Feraheme (BFA), which has been conjugated with a glutamate derivative (GLU) that targets PSMA in prostate cancer cells, show that this formulation is cytotoxic to PSMA positive prostate cancer cells (CWR22v1 and LNCaP) but not PSMA negative cancer cells (DU145 and PC3) (FIG. 24). In addition, cell adhesion studies show a time dependent detachment of LnCaP treated cells, while no significant detachment was observed in the PC3 (FIG. 25). It has been extensively reported that BFA causes a decrease in cell detachment with eventual cell death in cancer cells and our results show that when BFA is encapsulated into Feraheme, similar results are observed. This indicate that the encapsulation of BFA into Feraheme does not affect its ability to affect cancer cell. Experiments using HM-Feraheme (BFA) are also performed.

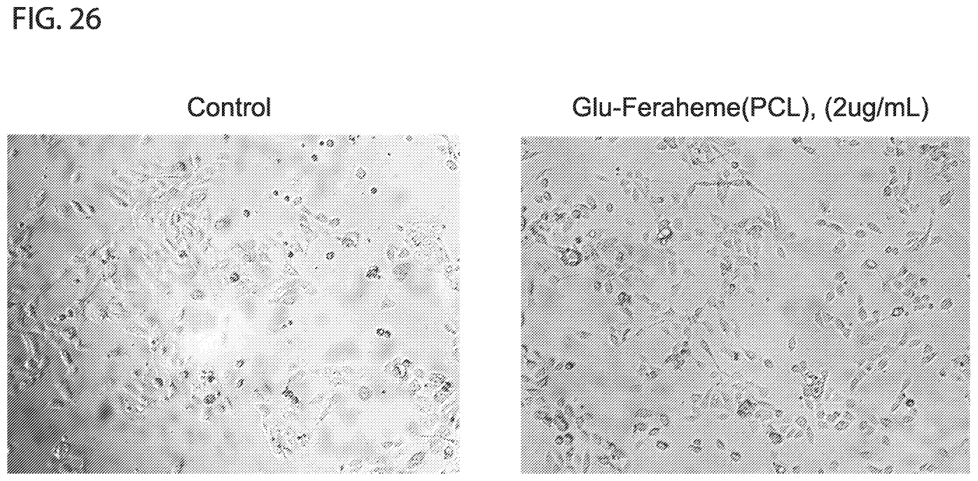

Most importantly, when normal prostate epithelial cells (RWPE) were treated with Glu-Feraheme (BFA), no significant change in cell morphology and cytotoxicity were observed (FIG. 26). Without being bound by theory, this seems to indicate that the Glu-Feraheme (BFA) formulation affect cancer cell more than normal cells.

In summary, a Feraheme formulation that target prostate cancer via PSMA has been developed for both imaging of prostate cancer via MRI or treatment of prostate cancer by delivering BFA to prostate cancer. As PSMA is not only expressed within prostate cancer but also on the neovasculature of other solid tumors. This invention can be expanded to the treatment of other solid tumors such as those from breast, lung, pancreas, and brain among others that express PSMA in their neovasculature.

In addition, in various embodiments of the present invention a Feraheme (BFA) formulation is also included as this non-targeted formulation can accumulate within tumors via the enhanced permeability and retention (EPR) effect and deliver TWA to tumors via this mechanism.

In various embodiments of the present invention, we have encapsulated Brefeldin A (BFA) in the polymeric coating of Feraheme (FH), Glutamate-Feraheme (GLU-FH), and Folate-Feraheme (FOL-FH) for the delivery of BFA into tumors. FH(BFA) can deliver the drug to tumors via the enhanced permeability and retention (EPR) effect, which is a passive and non-targeted way to deliver the drug. Meanwhile, GLU-FH and FOL-FH can be used to deliver the drug via the prostate specific membrane antigen (PSMA) which is overexpressed in prostate cancer and in the neovasculature of other solid tumors such as those of lungs, breast, pancreas, and brain (GBM).

### Example 10

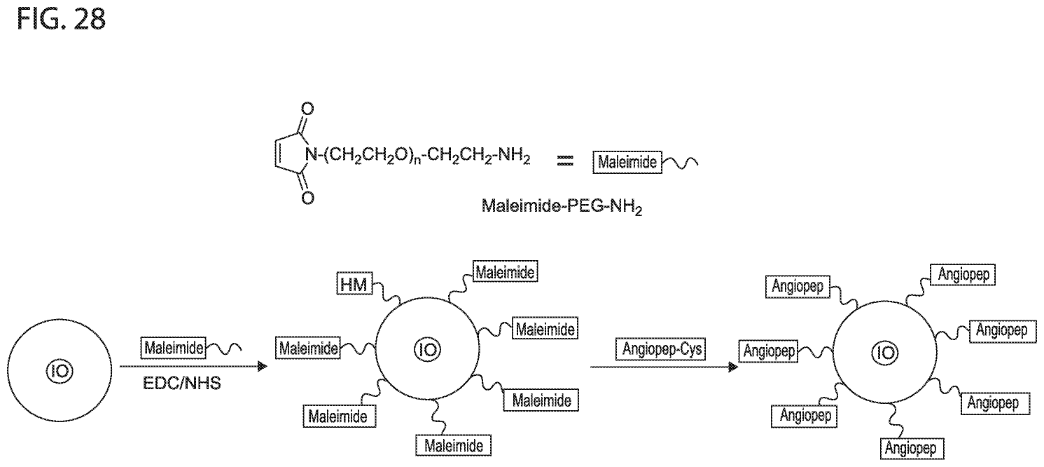

The Angiopep peptide was custom-ordered with a cysteine residue on the carboxylic acid end. (TFFYGGSRGKRNNFKTEEYC) (SEQ ID NO: 1) to facilitate binding to Feraheme via a maleimide linker. To achieve this, the carboxylic acid groups on Feraheme were first conjugated with a Maleimide-PEG-Amine linker using EDC/NHS ester chemistry and the resulting Maleimide-PEG-Feraheme was then reacted with the cysteine modified Angiopep (FIG. 28). The cysteine's sulfhydryl group on Angiopep exclusively reacts with the maleimide double bond forming a stable linker that conjugates Angiopep to the surface of Feraheme. The resulting Angiopep-Feraheme nanoparticles are characterized by DLS (size), and zeta potential (charge).

### Example 11

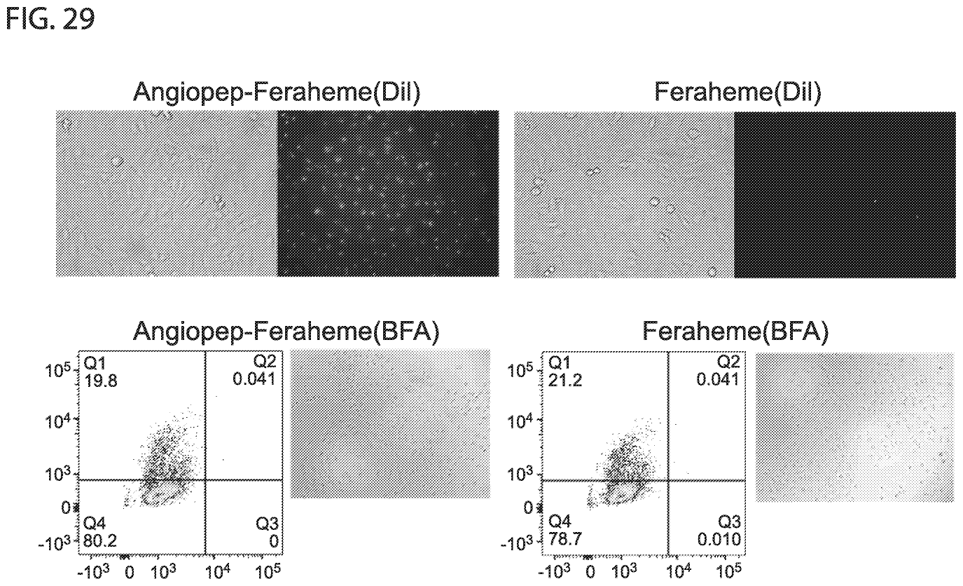

Association/Internalization of Angiopep-Feraheme into HBMVEC cells. We first studied the association and internalization of Angiopep-Feraheme (DiI) nanoparticles into human brain microvascular endothelial cells (HBMVEC). These cells are derived from the human brain vasculature and are used as a model to cross the BBB. In addition, they express LRP-1, the cell surface receptor target for Angiopep. So, without being bound by theory it was hypothesized that the nanoparticles with Angiopep would internalize into these cells. FIG. 29 show that indeed Angiopep facilitated the internalization of Feraheme into the cells. Notice that without Angiopep, the Feraheme (DiI) nanoparticles do not internalize into the HBMVEC cells. Furthermore, in the particular case a BFA a drug that affect protein transport in cancer cells, no toxicity is seen when the drug is encapsulated within Angiopep-Feraheme. FIG. 29 shows that when HBMVEC are treated with Angiopep-Feraheme (BFA), at a concentration of BFA of 550 nM, no significant chance in cytotoxicity is observed, as the percentage of viable cells of the treated vs the control (Feraheme (BFA)-treated)) is 80%. These results are important because the drug need to be cytotoxic to brain cancer cells and not to the brain vasculature of normal neurons. More studies with other normal brain cells are performed.

### Example 12

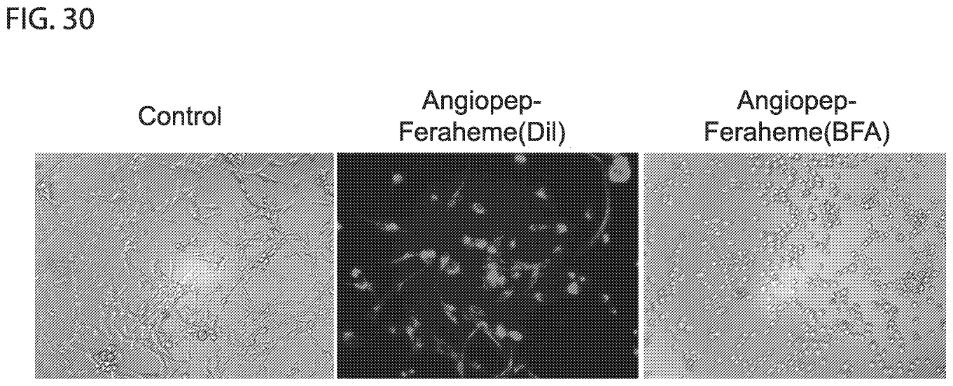

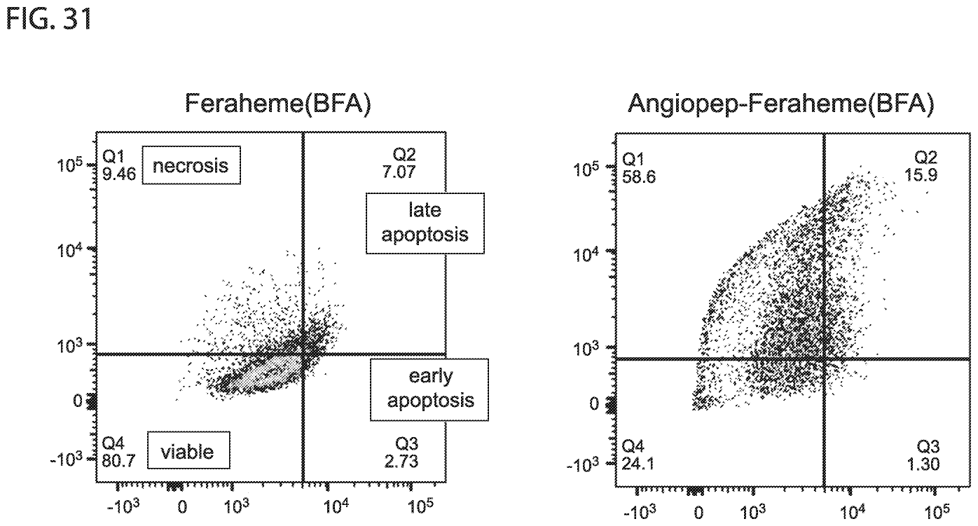

Internalization and effect of Angiopep-Feraheme (BFA) on U87 cells. U87 cells were used as a model system for GBM. It has been reported that LRP-1 is highly expressed in GBM. Results indicate that the nanoparticles internalize into U87, judged by the intense cell associated fluorescence of Angiopep-Feraheme (DiI) treated U87 cells (FIG. 30). In addition, when the Angiopep-Feraheme containing BFA are used, a dramatic change in cell morphology was observed within 48 hours that was associated with a dramatic reduction in cell viability (24% vs 80.7% in control cells) as see in Flow Cytometry studies (FIG. 31). These results clearly show that Angiopep is needed to facilitate internalization of the BFA carrying Feraheme nanoparticle to exert specific cytotoxicity to GBM cells via the LRp-1 receptor.

### Example 13

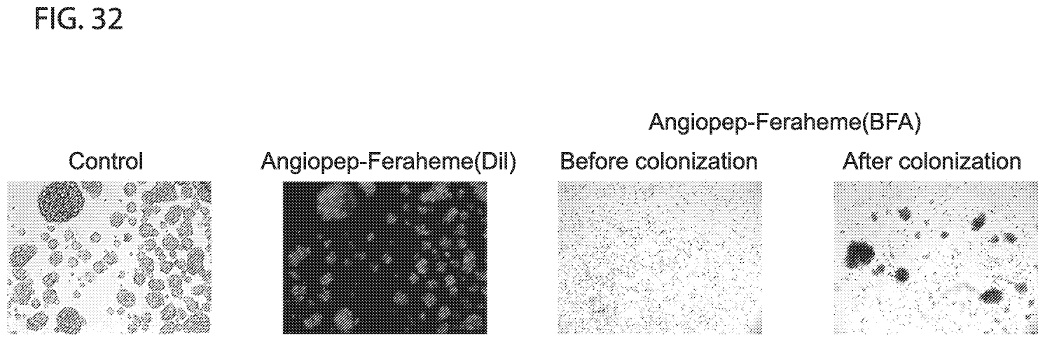

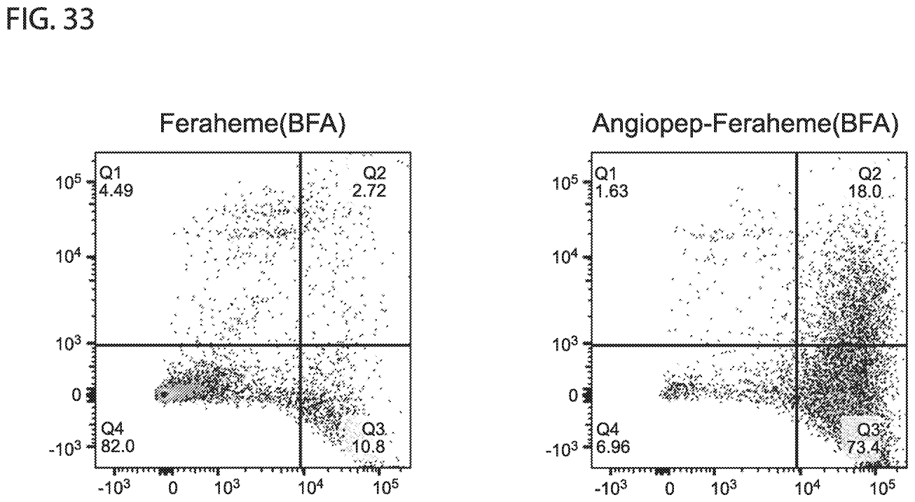

Internalization and effect on CSC55 GBM cells treated with Angiopep-Feraheme (BFA). To investigate if Angiopep-Feraheme can deliver a drug into GBM cancer stem cells, the CSC55 GBM stem cell line was first incubated with a version of the nanoparticles containing a fluorescent dye, Angiopep-Feraheme (DiI). Results show that upon 24 h incubation, significant cell associated fluorescence was observed (FIG. 32). Then, the Angiopep-Feraheme (BFA) formulation was incubated with the CSC55 cells at a final concentration of 550 nM of BFA, either right before colonies started to form and after the colonies were formed. Results show that when the cells were treated right before colonization, Angiopep-Feraheme (BFA) inhibited the formation of colonies even after 10 days of observation. Meanwhile, when cells were treated after the formation of visible colonies, the colonies reduced their size, and numbers. Also, a significant number of free cells in suspension was observed. In addition to a reduction in numbers, the morphology of the tumorspheres changed upon treatment (FIG. 32) Furthermore, a significant decrease in cell viability was observed in the cells treated after colonization (6.96% for the Angiopep-Feraheme (BFA) treated as opposed to the Feraheme (BFA) treated cells as control, 82%).

### Example 14

Heptamethine-Feraheme Conjugate for Dual Fluorescent and MRI Detection of Tumors and Drug Delivery. In various embodiments, the present invention relates to the use of conjugates of iron oxide nanoparticles with heptamethine dyes for the multimodal detection of tumors. Multimodal being defined as the ability of an agent to be detected in tissue by two imaging modalities, such as magnetic resonance imaging (MRI) and near infrared fluorescence (NIRF).

In various embodiments, the present invention is composed of a superparamagnetic iron oxide core of 2-8 nm coated with a carboxymethyl dextran polymer for a total nanoparticle size of 20-30 nm. The polymer coating stabilizes the iron oxide core to make the nanoparticle more biocompatible. The superparamagnetic properties of the iron oxide core create a locally induced magnetic field that diphase the spin of water molecules adjacent to the nanoparticle therefore creating a signal by MRI. A commercial and FDR-approved formulation of carboxymethyl dextran nanoparticles, Feraheme (Ferumoxytol), primarily used to treat iron deficiency (anemia), but increasingly used in MR-angiography and liver imaging was used as a polymer coated iron oxide nanoparticle.

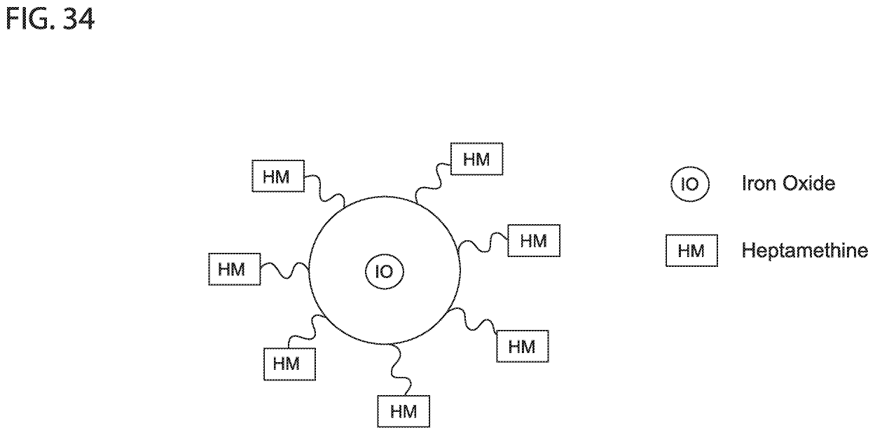

In various embodiments of the present invention, the carboxylic acid groups on the surface of the Feraheme nanoparticles were conjugated with a near-infrared heptamethine carbocyanine dye (HM) (FIG. 34). HM is a novel class of near-infrared fluorescent dye that is taken up by cancer cells via the organic anion transporting polypeptide (OATP), which is overexpressed in cancer cells. The novelty of HM is that it functions as both a near infrared fluorescence dye, capable of deep tissue imaging, and also a targeting ligand by itself to the OATP receptor in cancer cells. This dual property of HM as a cancer-targeting ligand and near infrared fluorescent allow for specific targeting, internalization and accumulation of the dye in cancer cells. Without out being bound by theory, we hypothesized that by conjugating HM to Feraheme, a HM-Feraheme nanoparticle conjugate would be produced with the following properties: 1. Multimodality—the accumulation of the HM-Feraheme nanoparticles in tumors can be imaged by either MRI and/or fluorescence imaging; 2. Tumor selective targeting—the binding, internalization and accumulation within cancer cells in tumors via the OATP receptor, with minimal internalization within normal cells; 3. Theranostic—Dual therapy and diagnostic (imaging) properties when a therapeutic anticancer drug is encapsulated within the polymeric dextran coating of the nanoparticle.

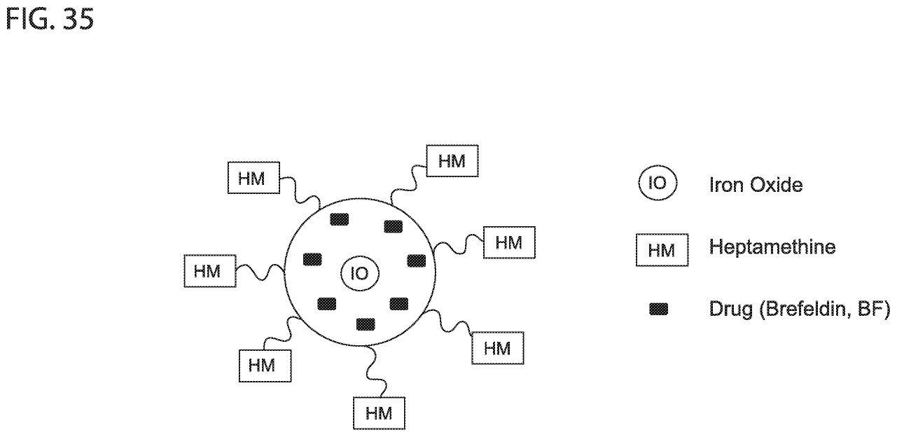

In various embodiments, the present invention provides a theranostic nanoparticle (FIG. 35) has been developed by encapsulating a drug within the carboxymethyldextran coating of the multimodal HM-Feraheme. We selected Brefeldin (BF) as a drug to encapsulate within Feraheme. Brefeldin, a promising drug patented by the NCI in 1997 (U.S. Pat. No. 5,696,154), has been extensively studied as an anticancer drug. Brefeldin inhibits protein trafficking and transport form the endoplasmic reticulum to the Golgi apparatus, causing activation of the unfolded protein response (UPR) and endoplasmic reticulum stress (ER-stress), which result in cell death by apoptosis. The known biological target of Brefeldin within the ER and ADP ribosylation factor 1 (ARF-1), a member of the RAS family of proteins that regulates the formation of protein transport vesicle within the ER. ARF-1 has been found to be elevated in various tumors and associated with invasion and metastasis. Therefore, ARF-1 in a good target for cancer therapy. A crystal structure of ARF-1 binding Brefeldin A has been reported. Brefeldin A has been shown to induce cell death by apoptosis or cell arrest in various cancer cell lines of leukemia, breast, colon, prostate, lung and brain, among others. In particular, it has been shown to inhibit the growth and migration of cancer stem cell. Unfortunately, the hydrophobic (water-insoluble) nature of this drugs hampers its successful intravenous administration to maintain therapeutic plasma concentrations that effectively kill tumors with minimal side effects. Therefore, novel ways to administer and target Brefeldin A to tumors are needed.

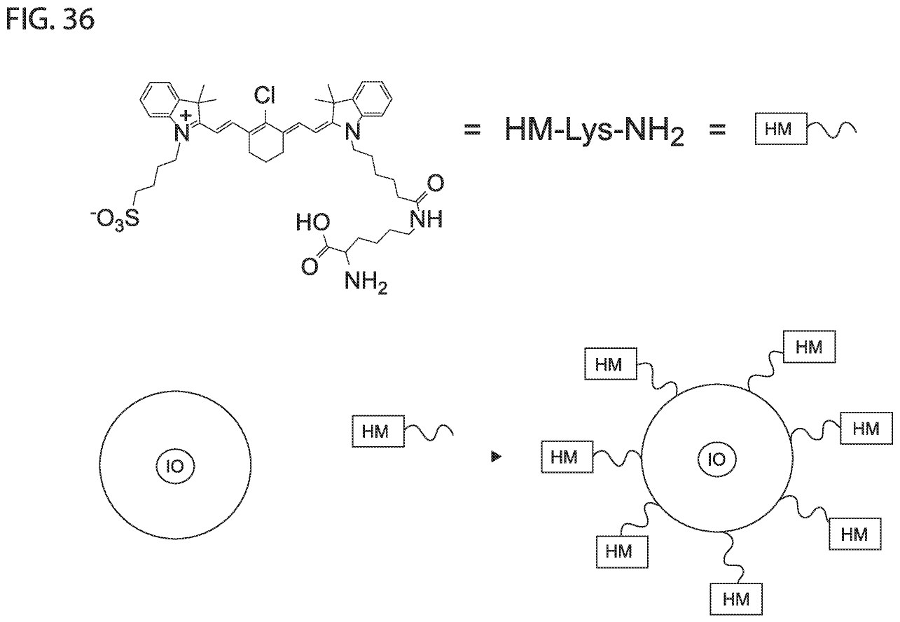

Multimodal HM-Feraheme Nanoparticle. A heptamethine-lysine conjugate (HM-Lys-NH₂) was synthesized. The amine group (—NH₂) on the lysine amino acid group was conjugated with the carboxylic acid group (—COOH) on the carboxymethyldextran coated of Feraheme using EDC/HNS chemistry. This resulted in conjugation of multiple heptamethine dyes to the surface of Feraheme via a lysine flexible linker (FIG. 36). The resulting HM-Feraheme nanoparticles are characterized by DLS (size), zeta potential (charge), and fluorescence spectroscopy. These nanoparticle-conjugates are stable, highly fluorescent and no loss of their magnetic properties is expected.

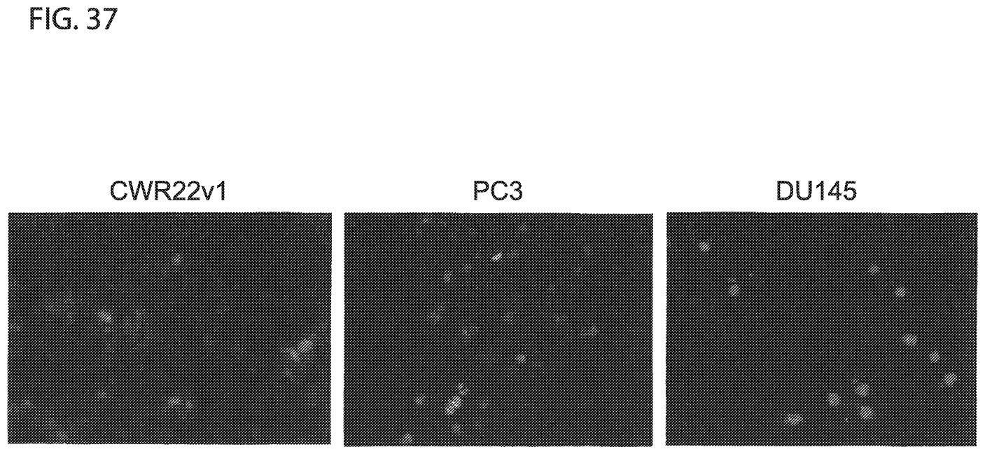

Preliminary Cell Internalization Studies. To study the ability of the HM-Feraheme nanoparticles to internalize and fluorescently label cancer cells, we treated various prostate cancer cell lines with the nanoparticles (1 ug/uL HM dye, 0.3 ug/uL Fe) for 12 h. Cells were imaged using near infrared fluorescence imaging. Results showed cell associated fluorescence in all cell, particularly CWR22v1, a cell line known to have increased levels of OATP (FIG. 37). Less fluorescence was observed in the PC3 and DU145 (FIG. 37). Without being bound by theory, it is not known if the lower fluorescence in PC3 and DU145 is due to lower expression of the OATP receptor on these cell lines.

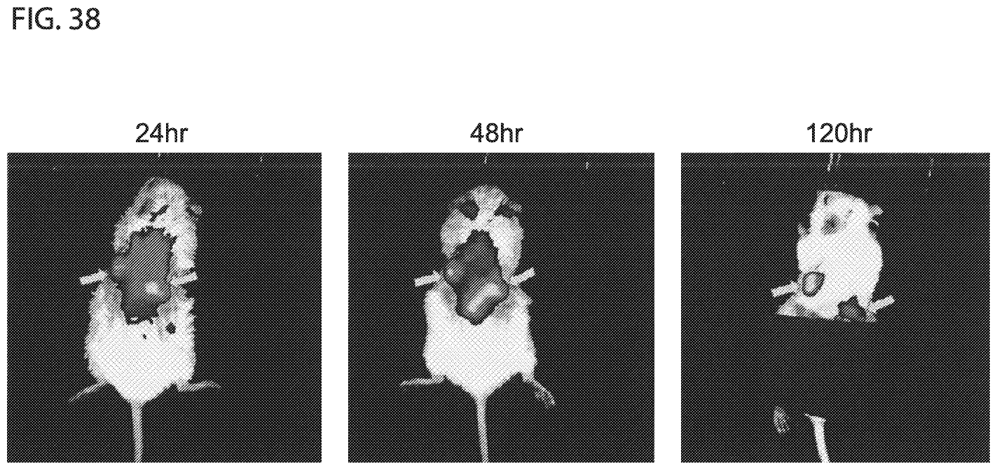

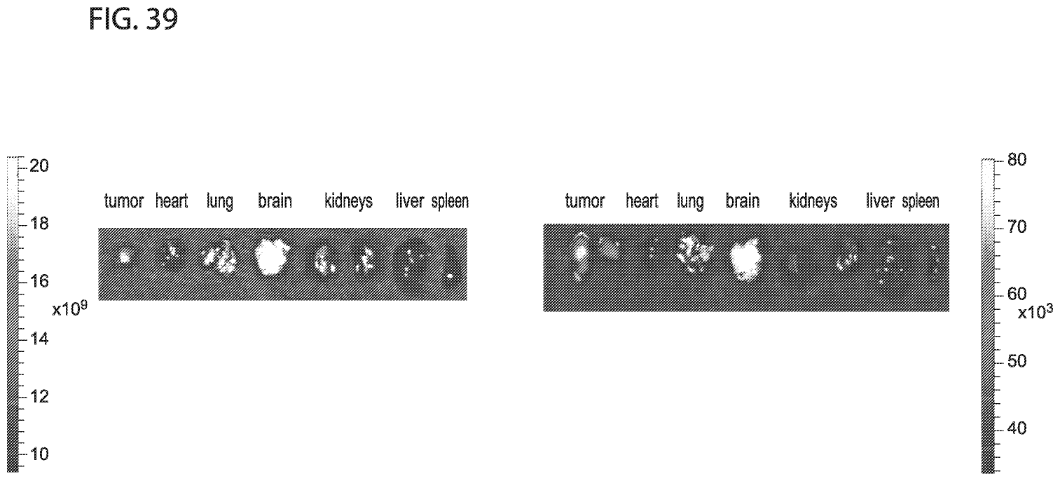

Preliminary in vivo studies. For these studies, 2 NSG mice were implanted with prostate cancer cells (CWR22Rv1) to develop prostate cancer tumor xenographs. The tumors were allowed to grow for 2 weeks before the mice were injected with 30 uL of HM-Feraheme (2 nmoles BM dye, 34 ug Fe). The animals were imaged using mouse fluorescence imaging after 24, 48 and 120 hr. FIG. 38 shows the results of one of those mice experiments. Notice that within 24 h, intense fluorescence is already observed within the implanted prostate cancer tumors. This tumor associated fluorescence remains in the tumors even after 120 hr. After 120 hr the animals were sacrificed and organs extracted and imaged. Results show strong near infrared fluorescence associated with the tumors with no detectable fluorescence in the rest of the organs (FIG. 39).

Theranostics HM-Feraheme (BF) Nanoparticle. After encouraging results obtained with targeting the HM-Feraheme nanoparticle to tumors, without being bound by theory we hypothesized that encapsulation of a therapeutic cargo (drug) would be feasible, achieving a theranostics (therapy and diagnostic[imaging]) nanoagent toward cancer. This would allow the monitoring of drug delivery by MRI and NIRF.

We have successfully encapsulated BFA on Feraheme to yield a Feraheme (BFA) preparation that is stable. We have prepared these formulations multiple times and the encapsulation procedure is reproducible. Encapsulation of BFA into Feraheme does not affect its stability, or particle size.

In various embodiments, the present invention provides for the pre-operative identification of tumor margins by magnetic resonance imaging, and during surgery using fluorescence imaging guided surgery. In various embodiments, the present invention provides for the tumor-targeted delivery of drugs using an iron oxide (e.g., Feraheme) formulation that targets OATP receptors in cancer cells and visualization of drug delivery by magnetic resonance imaging (MRI) or fluorescence imaging. In some embodiments, the fluorescence imaging is selected from the group consisting of near infrared fluorescence imaging, intraoperative fluorescence imaging, and combinations thereof.

In some embodiments, the present invention can be offered to cancer patients undergoing chemotherapy. For example, Feraheme is currently administered in the clinic for the treatment of anemia at a dose of 510 mg, followed by a second administration within 3-8 days. Without being bound by theory, for imaging and drug delivery purposes, a lower amount may be able to be used. In some embodiments, during chemotherapy, a once or twice a month administration of the nanoparticles, probes, or pharmaceutical composition thereof may be used. In some embodiments, for diagnostics and the assessment of tumor margins before and during surgery a one-time dose may be used.

### Example 15

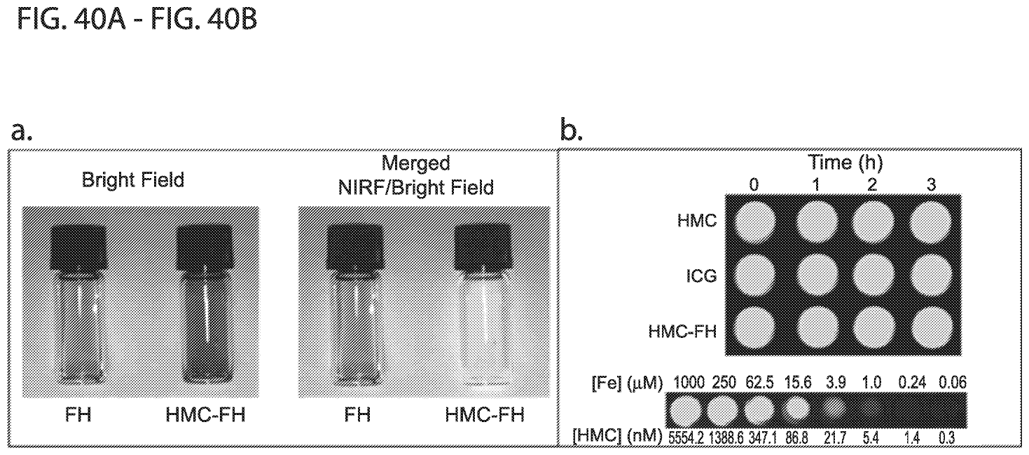

HMC-FH is a sensitive near infrared fluorescent nanoprobe that target GBM cells via OATP. Considering its exquisite tumor affinity and desirable NIRF properties, HMC was conjugated to FH for the fluorescent intraoperative detection of GBM tumors using the SIRIS system. This was achieved by modifying HMC with a lysine linker to yield HMC-Lys that is then conjugated onto the carboxylic acid groups in FH's carboxymethyl dextran coating via EDC chemistry. A lysine linker was selected because it increased HMC aqueous solubility, further facilitating conjugation and increasing nanoparticle aqueous solubility. Conjugation of HMC to FH does not affect its fluorescent properties, which are similar to those of ICG (FIG. 40A, FIG. 40B). Therefore, current imaging devices to detect ICG in clinical setting should work in detecting HMC and HMC-FH. The resulting HMC-FH is stable in aqueous buffers with intense near infrared fluorescence. HMC conjugation does not significantly affect the size (35.0±2.9 nm), zeta potential (−11.8±0.5), polydispersity (0.31±0.06) or stability of the nanoparticles in aqueous buffers. Upon excitation at 785 nm using the SIRS camera, bright and stable NIR fluorescence is observed even after intermittent illumination for 3 hours, with a limit of detection in the low nM range (FIG. 40B).

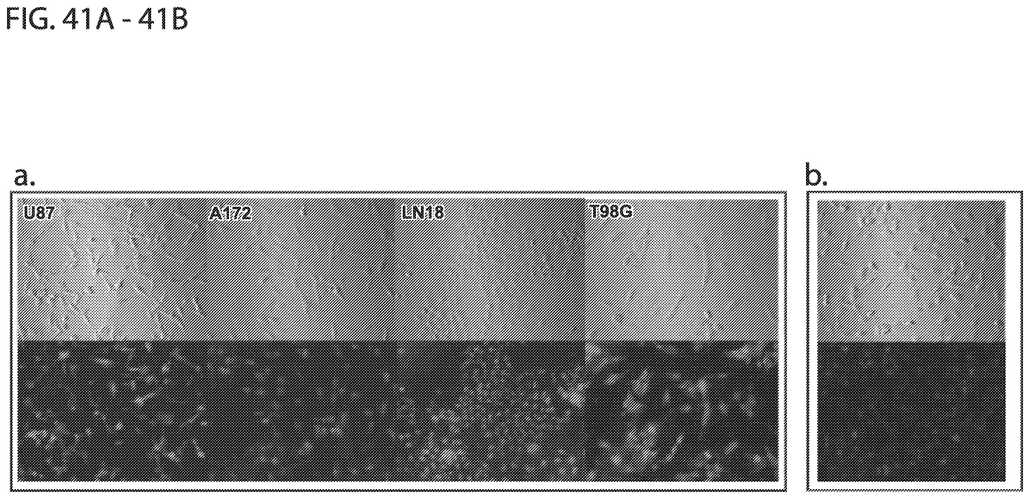

Next, we tested the ability of HMC-FH to target and internalize into various GBM cancer cell lines. For these studies, four cell lines (U87, A172, LN18, T98G) were incubated with HMC-FH (100 nm) for 24 H and imaged using a fluorescence microscopy. Results show bright MR fluorescence in the cytoplasm of all the cells studied indicating successful internalization of the HMC-FH (FIG. 41A). When the U87 cells were pre-incubated with Atazanir, a known OATP inhibitor, before incubation with HMC-FH, uptake and cell associated fluorescence was reduced by fluorescence microscopy and flow cytometry (FIG. 41B). Similar reduction in cell associated fluorescence was observed in cell pre-incubated with other OATP inhibitors such as Telmisartan, and Rifamicin. These results indicate that HMC-FH is internalized by various GBM cells via the OATP transporter. Furthermore, pre-incubation of the cells with sodium azide and 2-deoxyglucose, two known inhibitors of ATP-driven endocytosis, do not block the internalization of HMC-FH into GBM cells (data not shown). These results further corroborate the ATP-independent transport of HMC-FH into GBM cells via OATP.

### Example 16

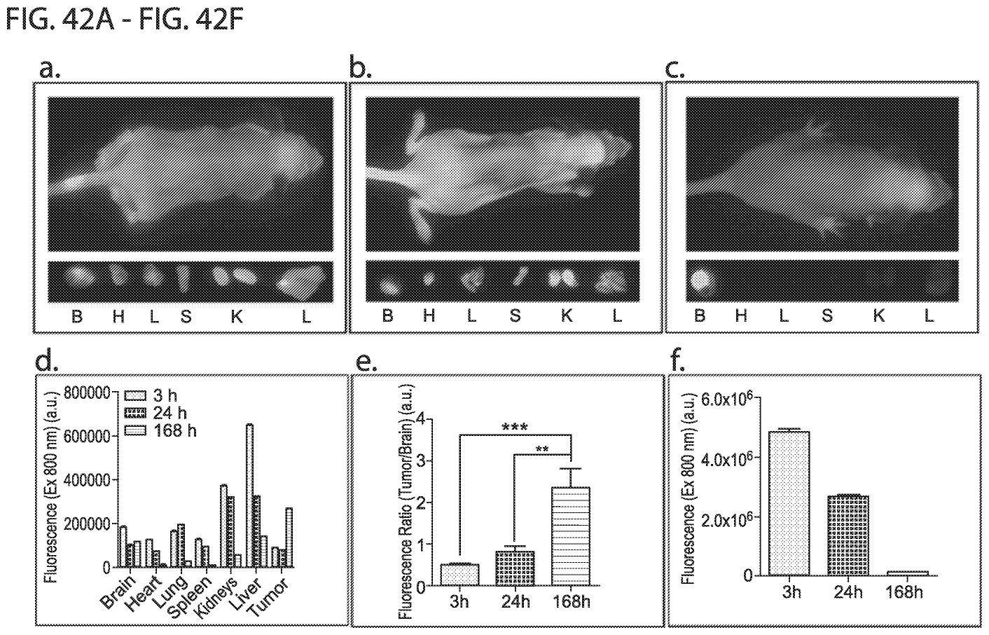

HMC-FH specifically localize and fluorescently label GBM tumors in an intracranial U87MG mouse model. To demonstrate HMC-FH ability to localize to tumor in an intracranial U87MG GBM mouse model, HMC-FH (1 mg HMC and 4 mgFe/kg mice) was injected i.v and mice imaged using SIRIS system 3, 24 or 168 h after injection. As soon as 3 h after injection of HMC-FH, fluorescence can be seen in the U87MG tumor within the mouse brain, as well as in other major organs (FIG. 42A). Similar results are observed in mice imaged 24 h after injection (FIG. 42B), although the tumor within the mouse brain is more visible at this time point. Surprisingly, the tumor associated fluorescence remained 168 h (1-week) after injection (FIG. 42C), indicating not only targeting but also stable retention of the HMC-FH. After a week, fluorescence is not observed in most major organs, suggesting clearance from these organs. Quantification of the fluorescence associated with each of the organs shows a sequential decrease in fluorescence with time, except in the GBM tumor where a large increase in tumor associated fluorescence is seen within a week (FIG. 42D). The observed increase in fluorescence intensity correlates with an increase in the calculated tumor-to-brain fluorescent intensity value (FIG. 42E), indicating that the tumor associated fluorescence increases, while decreasing in the healthy brain tissue. Finally, preliminary pharmacokinetic studies show that the presence of HMC fluorescence in the blood decreases with time, with the lowest value observed within a week (FIG. 42F). Taken together, these results show that HMC-FH targets and strongly associates with GBM tumors in an intracranial mouse tumor model. The fact that a strong GBM tumor associated fluorescence remains even after 1-week suggests that, if implemented in the clinic, neurosurgeon would have more flexibility to perform the surgery, between 1 or 7 days after HMC-FH administration.

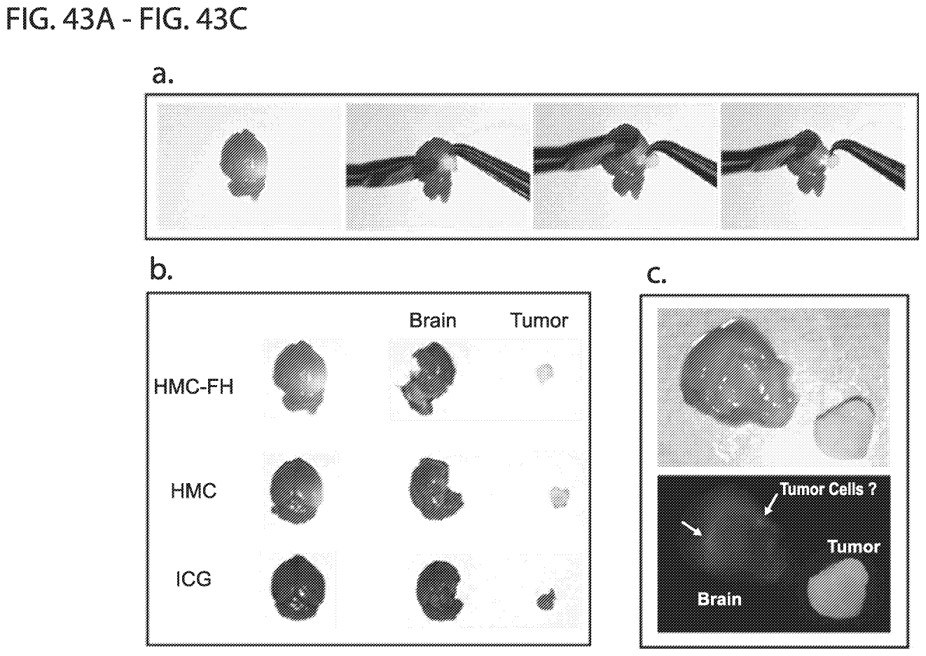

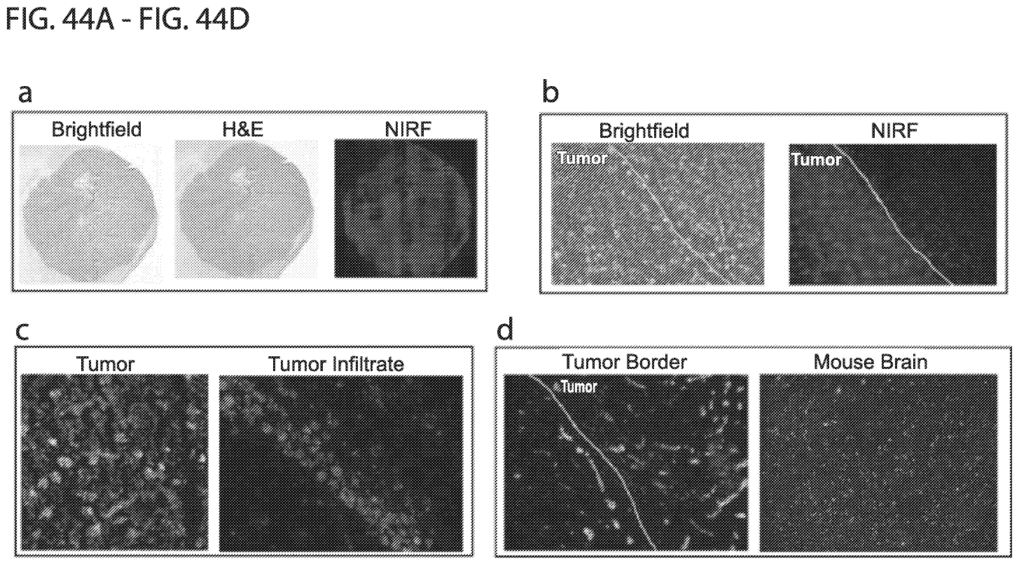

The successful association and corresponding near infrared fluorescent labeling of GBM tumors by HMC-FH, suggest that HMC-FH can aid in the visualization of these tumor intraoperatively when used in combination with SIRIS or any other intraoperative fluorescent camera. To prove this capability in a mouse model, a “mock” surgery was performed on mice brains with intracranial U87MG tumors. In these experiments, mice with intracranial GBM tumors were injected with HMC-FH, HMC or ICG and euthanized 24 h after injection. Mouse brains were extracted from the mouse skull, and tumors visualized and resected from the healthy brain while recording using SIRIS. This approach was chosen because the survival of mice undergoing brain intraoperative surgery is poor, with a very low number of mice surviving the procedure. We selected to perform the “mock surgery” 24 hours after administration of HMC-FH, because at this early time point enough fluorescent signal is observed in the tumor to facilitate successful resection. FIG. 43A show movie snapshots of the procedure, where the strong fluorescence in the tumor facilitates the complete extraction of the small tumors (FIG. 43A). After extraction, no detectable fluorescence is seen in the “healthy” brain tissue, suggesting complete resection of the tumor mass as indicated by fluorescence imaging (FIG. 43B). Similar results were obtained with the HMC dye along and show that the cancer targeting ability of HMC is not compromised in HMC-FH. In contrast, ICG does not show tumor localization as the extracted tumor is not fluorescently labeled. When a larger and infiltrating GBM tumor was imaged, fluorescence was seen not only in the extracted tumor, but also in regions near the “surgical” cavity suggesting the presence of infiltrating tumor tissue (FIG. 43C). This will need to be corroborated by histopathogy (see FIG. 44C, Tumor Infiltrate). Taken together, these preliminary studies show the feasibility HMC-FH to fluorescently label GBM tumors, identifying tumor margins and facilitating their surgical extraction.

### Example 17

HMC-FH crosses the BBB and bind to tumor cells in an intracranial GBM mouse model. Visualization of the fluorescent-labeled brain tissue by microscopy (brighfield) and histopathology (H&E staining) corroborates the existence of tumor tissue associated with the observed near infrared fluorescent (FIG. 44A). Upon magnification of the tumor border area, fluorescence is observed in the tumor cells, indicating localization and uptake of HMC-FH by the U87MG cells (FIG. 44B). Minimal fluorescence is observed in the cells adjacent to the tumor. Identification of the human U87MG cells in the tumor with an antibody that recognizes human nestin (a known neuronal marker, green in FIG. 44C) shows that the fluorescent HMC-FH nanoparticles (red in FIG. 44C) associates with the U87MG cells in the tumor as well as in the tumor infiltrate. In contrast, the HMC-FH fluorescence signal (red in FIG. 44D does not co-localize with the vascular endothelium (green in FIG. 44D), indicating that the HMC-FH have crossed the BBB. Together, these results indicate that HMC-FH crosses the BBB in the tumor area and bind to GBM cancer cells in an intracranial mouse tumor model.

### Example 18

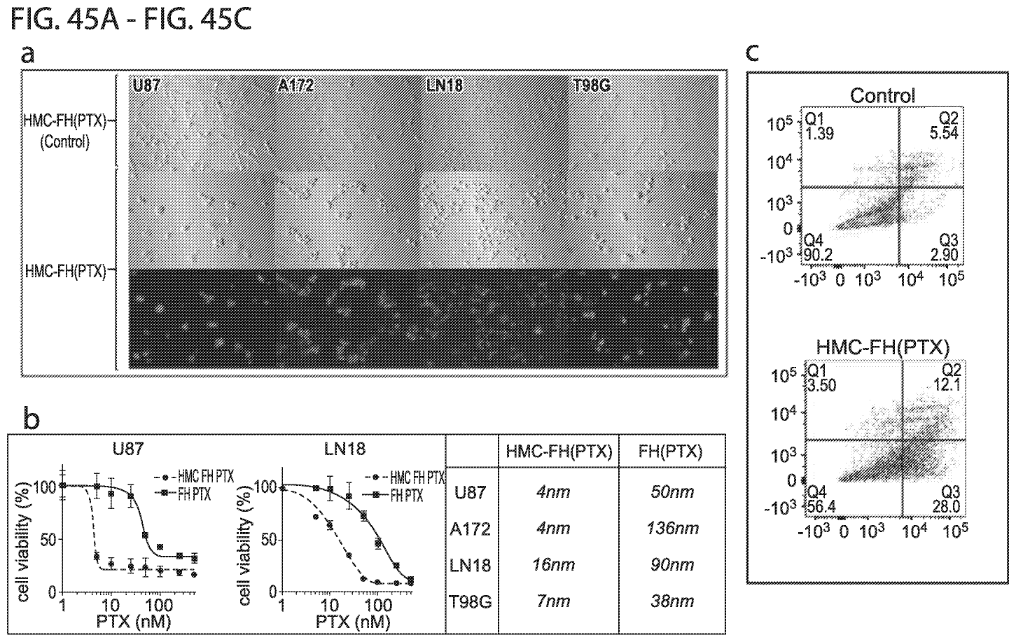

HMC-FH can deliver a drug to GBM cells in culture and GBM tumors in a mouse intracranial model, reducing tumor growth and increasing survival. The fact that HMC-FH can associate with GBM cell in a intracranial mouse tumor model suggest that HMC-FH can be used to deliver drugs post-surgery. Our published preliminary data shows that FH can deliver drugs to subcutaneous tumors in mice, reducing tumor volume (FIG. 45A). For the HMC-FH assisted treatment of GBM, we have selected paclitaxel as our model drugs. Paclitaxel is one of the most effective chemotherapeutics against cancer, proving to be highly effective in the treatment of solid tumors such as those from breast, and lung. However, its used in the treatment of gliomas have been limited due to the poor BBB-crossing ability of this drug. Our current data shows that HMC-FH can encapsulate PTX and that this encapsulation does not affect the size (35±2.9 nm), zeta potential (−12.1±0.5), polydispersity (0.31±0.06), or stability of the nanoparticles. The current PTX encapsulation efficacy is 66±0.1%. HMC-FH stably encapsulate PTX and other drug for months at 4 C in PBS, with accelerated drug release at body temperature (37 C) and slightly acid pH, 6.8, as it has been reported with other drugs. When various GBM cell lines were incubated with HMC-FH(PTX), significant changes in cell morphology were observed in 72 h (FIG. 45A) with an apparent IC50 values in the low nm range (FIG. 45B). PE-Annexin V/7-ADD flow cytrometric analysis of treated U87MG cells show decrease cell viability (56.4%) with corresponding increase in the number of early (28%) and late apoptotic cells (12.1), in contrast with non-treated control cells (90.2% viable cells).

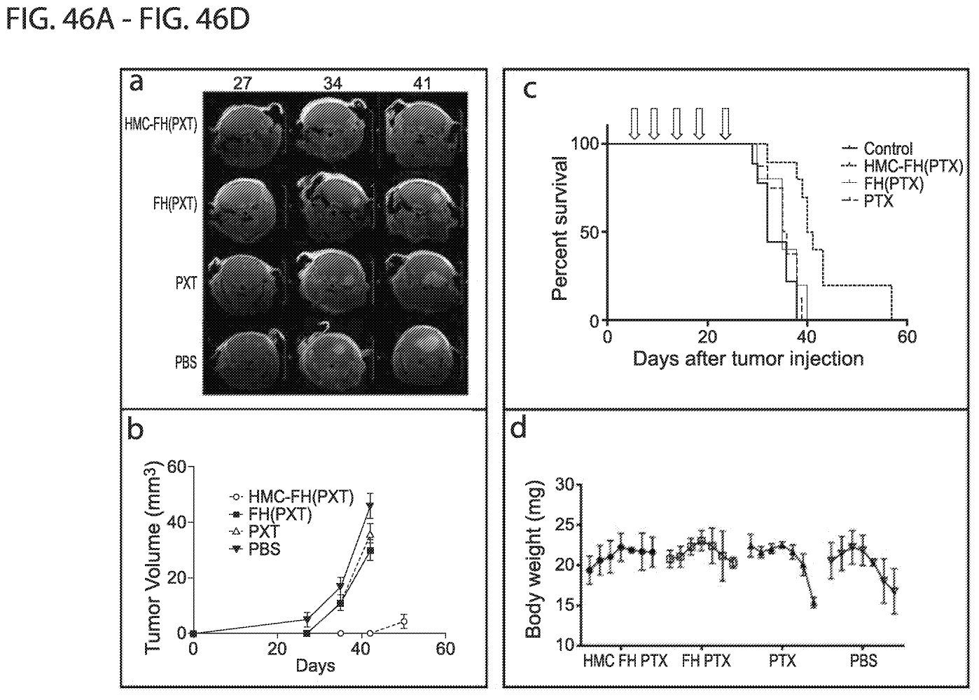

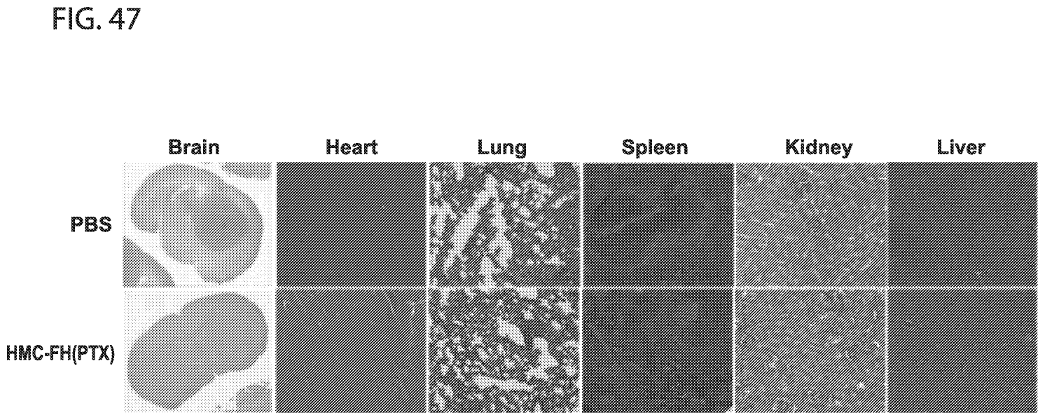

The HMC-FH(PTX) was next used to treat nude mice with human intracranial U87MG tumors. Six treatments (1 mg HMC, 3 mg PTX and 4 mgFe/kg mice) resulted in a dramatic reduction of the tumor growth, with no visible tumor detection by MRI (FIG. 46A, FIG. 46B), during the treatment period (40 days after tumor inoculation). In contrast, control mice and mice treated with FH(PTX) or PTX along developed visible tumor during the observation tumor. Results showed that both HMC-FH(PXT) was more efficient that the drug along in reducing the growth and size of the tumors. It is not until after the treatment period that tumors start developing in the HMC-FH(PTX) treated mice (FIG. 46B). In another set of experiments, mice similarly treated with HMC-FH, had a longer survival than control mice, or mice treated with FH(PTX) or PTX along (FIG. 46C). Mice treated with HMC-FH did not have weigh reduction (FIG. 46D). Histopathological studies of the isolated brain and other vital organs corroborate the absence of brain tumor in the HMC-FH(PTX) treated mice, with no visible damage to major organs (FIG. 47).

### Example 19

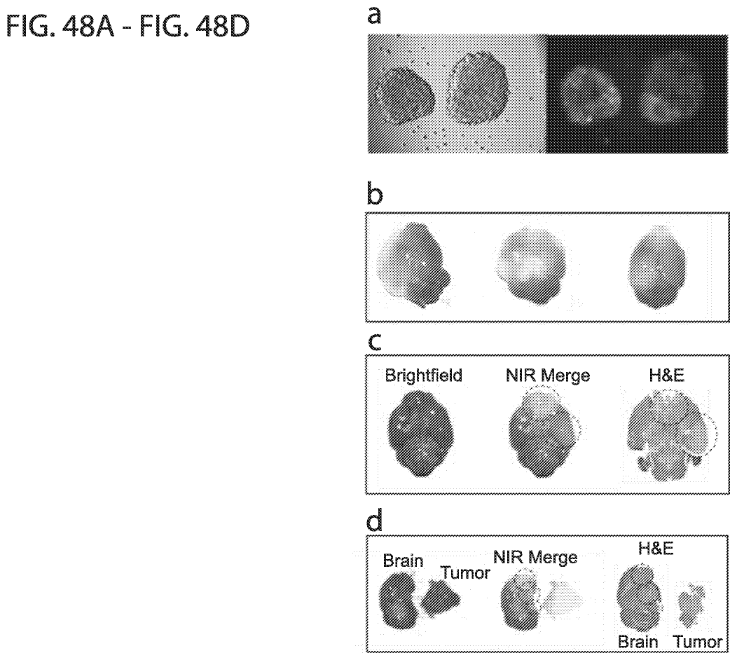

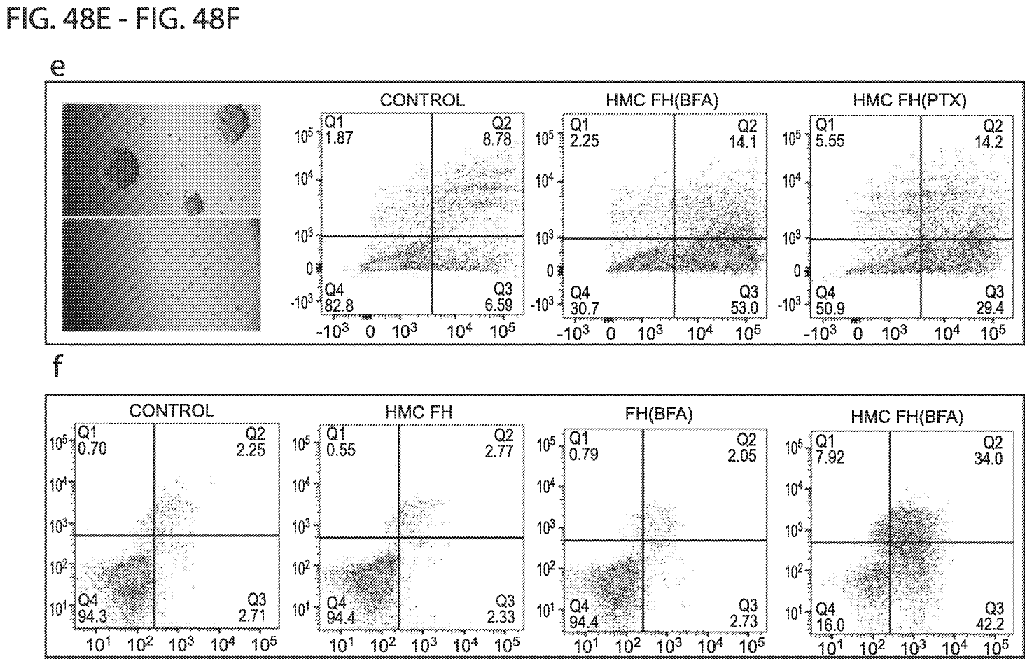

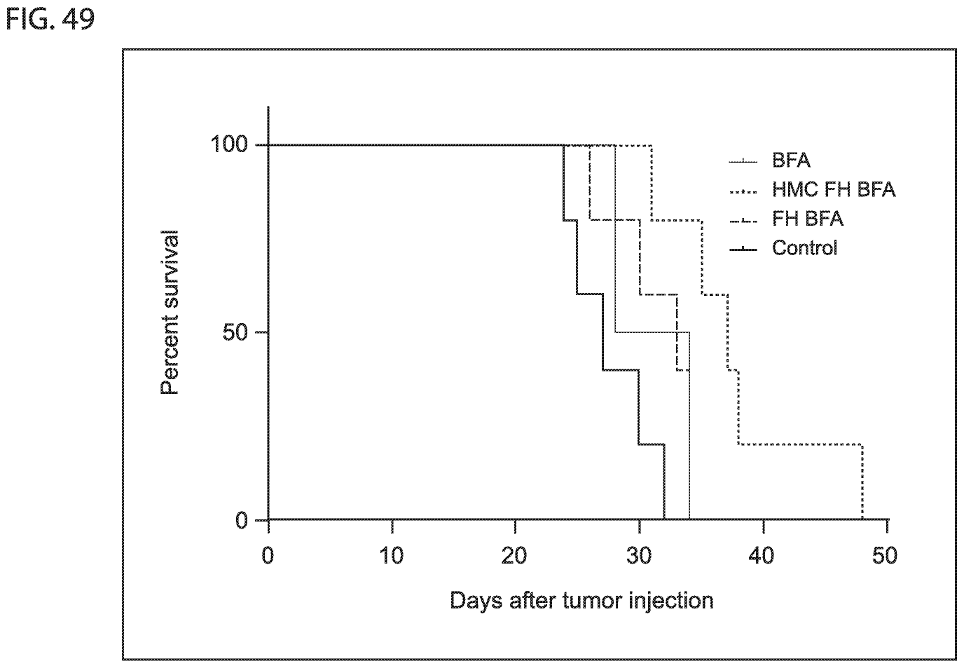

HMC-FH binds to Patient derived GBM Stem Cells and to intracranial tumor models generated using those cells. The complex genetic variability of GBM demand the use of reliable animal models that can better recapitulate the biology of GBM and better predict therapeutic outcome for individual patients. Toward this goal, we have established orthotopic (GBM) xenograft models using patient derived GBM Stem Cells. These patient derived GBM tumors better recapitulate both the infiltrating and migratory nature of GBM and maintain the genomic characteristics of human GBM. Therefore, we reasoned that the use of this mouse model to study HMC-FH ability to target GBM and assist during intraoperative surgery and as a drug delivery vehicle could better mimic and be more predictive of its future clinical application. First, we tested if these patient derived GBM stem cells were able to uptake HMC-FH and become fluorescence. Results show that these cells readily uptake and become brightly fluorescent upon exposure with HMC-FH (24 h) (FIG. 48A). When orthotopic (GBM) xenograft mouse models were generated using these cells, migratory and infiltrating brain tumors were generated in mice that were easily visualized by fluorescence imaging using SIRIS after HMC-FH administration (FIG. 48B). The fluorescently labeled areas within the mouse brain clearly correlate with the tumor area identified by H&E staining of the brain slides (FIG. 48C). Most importantly areas within the brain not fluorescently labeled by HMC-FH were identified as areas with no tumor. The precise localization of HMC-FH to only areas with tumor burden using this patient derived GBM stem cell model, while sparing brain healthy tissue, further indicates that HMC-FH could be successfully implemented in fluorescence intraoperative surgery of gliomas. FIG. 48D shows a resected GBM tumor using this model, where significant tumor infiltration is present near the brain tissue adjacent to the tumor. The specific association of HMC to infiltrating GBM tissue would aid the surgeon in further resecting more precisely all tumor tissue as well as in the delivery of drugs to GBM. Furthermore, the ability of HMC-FH to cross the BBB gives us the opportunity to deliver not only current chemotherapeutic drugs that typically do not cross the BBB but also novel drugs that kill cancer cells by unique mechanisms, particularly GBM cancer stem cell that are typically chemoresistant. Toward this goal, we incubated GBM stem cells with HMC-FH(PTX) or HMC-FH(BFA). Brefeldin A (BFA) biological effects and mechanism of action are well-documented in the literature. Brefeldin A (BFA) is a small macrocylic lactone which inhibits protein transport between the endoplasmic reticulum (ER) and the Golgi. This inhibition results in accumulation of proteins in the ER triggering activation of an unfolded protein response (UPR) and eventual ER stress, which results in cell death. In particular, BFA prevents the formation of transport vesicles that move proteins between the ER and Golgi by inhibition of ADP ribosylation factor (ARF1), a key regulator of vesicular formation and trafficking. As ARF1 is over expressed in various type of tumors, playing a key role in cell proliferation, invasiveness and progression, prevention of its activation by BFA represent a promising and novel approach to treat cancer. Unfortunately, systemic administration of this drug has been challenging due to its toxicity and its poor aqueous solubility which have hampered the development of clinical formulations. We have been successful in encapsulating BFA into HMC-FH and the resulting HMC-FH(BFA) used to treat GBM stem cells in culture. Results show that HMC-FH(BFA) kill the GBM cancer stem cells more efficiently than HMC-FH(PTX) (FIG. 48E, FIG. 48F). These results are highly significant as these cells are typically drug resistant and difficult to treat. Furthermore, HMC-FH(BFA) increase survival in orthotopic U87 GBM mice (n=5), with no detectable toxicity to the mice.

### REFERENCES

1. Verma, S., et al., The Current State of MR Imaging-targeted Biopsy Techniques for Detection of Prostate Cancer. Radiology, 2017. 285(2): p. 343-356.
2. Acar, C., et al., Advances in sentinel node dissection in prostate cancer from a technical perspective. Int J Urol, 2015. 22(10): p. 898-909.
3. Brouwer, O. R., et al., Image navigation as a means to expand the boundaries of fluorescence-guided surgery. Phys Med Biol, 2012. 57(10): p. 3123-36.
4. Cornejo-Davila, V., et al., Use of near infrared fluorescence during robot-assisted laparoscopic partial nephrectomy. Actas Urol Esp, 2016. 40(3): p. 190-4.
5. Greco, F., et al., Current perspectives in the use of molecular imaging to target surgical treatments for genitourinary cancers. Eur Urol, 2014. 65(5): p. 947-64.
6. Hall, M. A., et al., Imaging prostate cancer lymph node metastases with a multimodality contrast agent. Prostate, 2012. 72(2): p. 129-46.
7. Liss, M. A., et al., Robotic-assisted fluorescence sentinel lymph node mapping using multimodal image guidance in an animal model. Urology, 2014. 84(4): p. 982 e9-14.
8. Lutje, S., et al., Dual-Modality Image-Guided Surgery of Prostate Cancer with a Radiolabeled Fluorescent Anti-PSMA Monoclonal Antibody. J Nucl Med, 2014. 55(6): p. 995-1001.
9. Sonn, G. A., et al., Fluorescent Image-Guided Surgery with an Anti-Prostate Stem Cell Antigen (PSCA) Diabody Enables Targeted Resection of Mouse Prostate Cancer Xenografts in Real Time. Clin Cancer Res, 2016. 22(6): p. 1403-12.
10. Wang, X., et al., Development of targeted near-infrared imaging agents for prostate cancer. Mol Cancer Ther, 2014. 13(11): p. 2595-606.
11. Xia, L., et al., Near-infrared Intraoperative Molecular Imaging Can Identify Metastatic Lymph Nodes in Prostate Cancer. Urology, 2017. 106: p. 133-138.
12. Yuen, K., et al., Intraoperative Fluorescence Imaging for Detection of Sentinel Lymph Nodes and Lymphatic Vessels during Open Prostatectomy using Indocyanine Green. J Urol, 2015. 194(2): p. 371-7.
13. Litwin, M. S. and H. J. Tan, The Diagnosis and Treatment of Prostate Cancer: A Review. JAMA, 2017. 317(24): p. 2532-2542.
14. Auerbach, M., et al., Safety and efficacy of total dose infusion of 1,020 mg of ferumoxytol administered over 15 min. Am J Hematol, 2013. 88(11): p. 944-7.
15. Cheng, K., et al., Magnetic antibody-linked nanomatchmakers for therapeutic cell targeting. Nat Commun, 2014. 5: p. 4880.
16. Lu, M., et al., FDA report: Ferumoxytol for intravenous iron therapy in adult patients with chronic kidney disease. Am J Hematol, 2010. 85(5): p. 315-9.
17. Bashir, M. R., et al., Emerging applications for ferumoxytol as a contrast agent in MRI. J Magn Reson Imaging, 2015. 41(4): p. 884-98.
18. Hope, M. D., et al., Vascular Imaging With Ferumoxytol as a Contrast Agent. AJR Am J Roentgenol, 2015. 205(3): p. W366-73.
19. Toth, G. B., et al., Current and potential imaging applications of ferumoxytol for magnetic resonance imaging. Kidney Int, 2017. 92(1): p. 47-66.
20. Schwenk, M. H., Ferumoxytol: a new intravenous iron preparation for the treatment of iron deficiency anemia in patients with chronic kidney disease. Pharmacotherapy, 2010. 30(1): p. 70-9.
21. Santra, S., et al., Drug/dye-loaded, multifunctional iron oxide nanoparticles for combined targeted cancer therapy and dual optical/magnetic resonance imaging. Small, 2009. 5(16): p. 1862-8.
22. Kaittanis, C., et al., Environment-responsive nanophores for therapy and treatment monitoring via molecular MRI quenching. Nat Commun, 2014. 5: p. 3384.
23. Li, R., et al., Be Active or Not: the Relative Contribution of Active and Passive Tumor Targeting of Nanomaterials. Nanotheranostics, 2017. 1(4): p. 346-357.
24. Miller, M. A., S. Arlauckas, and R. Weissleder, Prediction of Anti-cancer Nanotherapy Efficacy by Imaging. Nanotheranostics, 2017. 1(3): p. 296-312.
25. Obaidat, A., M. Roth, and B. Hagenbuch, The expression and function of organic anion transporting polypeptides in normal tissues and in cancer. Annu Rev Pharmacol Toxicol, 2012. 52: p. 135-51.
26. Shi, C., J. B. Wu, and D. Pan, Review on near-infrared heptamethine cyanine dyes as theranostic agents for tumor imaging, targeting, and photodynamic therapy. J Biomed Opt, 2016. 21(5): p. 50901.
27. Roth, M., A. Obaidat, and B. Hagenbuch, OATPs, OATs and OCTs: the organic anion and cation transporters of the SLCO and SLC22A gene superfamilies. Br J Pharmacol, 2012. 165(5): p. 1260-87.
28. Buxhofer-Ausch, V., et al., Tumor-specific expression of organic anion-transporting polypeptides: transporters as novel targets for cancer therapy. J Drug Deliv, 2013. 2013: p. 863539.
29. Pressler, H., et al., Expression of OATP family members in hormone-related cancers: potential markers of progression. PLoS One, 2011. 6(5): p. e20372.
30. Yuan, J., et al., Nearinfrared fluorescence imaging of prostate cancer using heptamethine carbocyanine dyes. Mol Med Rep, 2015. 11(2): p. 821-8.
31. Shi, C., et al., Heptamethine carbocyanine dye-mediated near-infrared imaging of canine and human cancers through the HIF-1alpha/OATPs signaling axis. Oncotarget, 2014. 5(20): p. 10114-26.
32. Yang, X., et al., Optical imaging of kidney cancer with novel near infrared heptamethine carbocyanine fluorescent dyes. J Urol, 2013. 189(2): p. 702-710.
33. Yang, X., et al., Near IR heptamethine cyanine dye-mediated cancer imaging. Clin Cancer Res, 2010. 16(10): p. 2833-44.
34. Wu, J. B., et al., Near-infrared fluorescence imaging of cancer mediated by tumor hypoxia and HIF1alpha/OATPs signaling axis. Biomaterials, 2014. 35(28): p. 8175-85.
35. Miller, K. D., et al., Cancer treatment and survivorship statistics, 2016. CA Cancer J Clin, 2016. 66(4): p. 271-89.
36. Siegel, R. L., K. D. Miller, and A. Jemal, Cancer Statistics, 2017. CA Cancer J Clin, 2017. 67(1): p. 7-30.
37. Petrylak, D. P., et al., Docetaxel and estramustine compared with mitoxantrone and prednisone for advanced refractory prostate cancer. N Engl J Med, 2004. 351(15): p. 1513-20.
38. Tannock, I. F., et al., Docetaxel plus prednisone or mitoxantrone plus prednisone for advanced prostate cancer. N Engl J Med, 2004. 351(15): p. 1502-12.
39. Krex, D., et al., Long-term survival with glioblastoma multiforme. Brain, 2007. 130(Pt 10): p. 2596-606.
40. Stupp, R., et al., Radiotherapy plus concomitant and adjuvant temozolomide for glioblastoma. N Engl J Med, 2005. 352(10): p. 987-96.
41. Stupp, R., et al., Maintenance Therapy With Tumor-Treating Fields Plus Temozolomide vs Temozolomide Alone for Glioblastoma: A Randomized Clinical Trial. JAMA, 2015. 314(23): p. 2535-43.
42. Bao, S., et al., Glioma stem cells promote radioresistance by preferential activation of the DNA damage response. Nature, 2006. 444(7120): p. 756-60.
43. Rich, J. N. and S. Bao, Chemotherapy and cancer stem cells. Cell Stem Cell, 2007. 1(4): p. 353-5.
44. Rycaj, K. and D. G. Tang, Cancer stem cells and radioresistance. Int J Radiat Biol, 2014. 90(8): p. 615-21.
45. Tamura, K., et al., Expansion of CD133-positive glioma cells in recurrent de novo glioblastomas after radiotherapy and chemotherapy. J Neurosurg, 2013. 119(5): p. 1145-55.
46. Fujiwara, T., et al., Brefeldin A causes disassembly of the Golgi complex and accumulation of secretory proteins in the endoplasmic reticulum. J Biol Chem, 1988. 263(34): p. 18545-52.
47. Lee, S. A., Y. J. Kim, and C. S. Lee, Brefeldin a induces apoptosis by activating the mitochondrial and death receptor pathways and inhibits focal adhesion kinase-mediated cell invasion. Basic Clin Pharmacol Toxicol, 2013. 113(5): p. 329-38.
48. Lippincott-Schwartz, J., et al., Rapid redistribution of Golgi proteins into the ER in cells treated with brefeldin A: evidence for membrane cycling from Golgi to ER. Cell, 1989. 56(5): p. 801-13.
49. Pommepuy, I., et al., Brefeldin A induces apoptosis and cell cycle blockade in glioblastoma cell lines. Oncology, 2003. 64(4): p. 459-67.
50. Cherfils, J. and P. Melancon, On the action of Brefeldin A on Sec7-stimulated membrane-recruitment and GDP/GTP exchange of Arf proteins. Biochem Soc Trans, 2005. 33(Pt 4): p. 635-8.
51. Schlienger, S., et al., ADP-ribosylation factor 1 expression regulates epithelial-mesenchymal transition and predicts poor clinical outcome in triple-negative breast cancer. Oncotarget, 2016. 7(13): p. 15811-27.
52. Chen et al., Angiopep-pluronic F127-conjugated superparamagnetic iron oxide nanoparticles as nanotheranostic agents for BBB targeting. J. Mater. Chem. B, 2014. 2: p. 5666-5675.

Various methods and techniques described above provide a number of ways to carry out the application. Of course, it is to be understood that not necessarily all objectives or advantages described can be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

Various embodiments of this application are described herein, including the best mode known to the inventors for carrying out the application. Variations on those embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting affect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

1. A nanoparticle, comprising:
a core, wherein the core comprises at least one iron oxide;
a shell surrounding the core, wherein the shell comprises at least one polymer; and
at least one targeting moiety attached to the shell,
wherein the nanoparticle does not comprise boron.

2. The nanoparticle of claim 1, wherein the at least one iron oxide is selected from the group consisting of FeO, Fe₂O₃, and combinations thereof.

3. The nanoparticle of claim 1, wherein the at least one polymer is at least one biocompatible polymer or at least one polysaccharide.

4. (canceled)

5. The nanoparticle of claim 1, wherein the at least one polymer is selected from the group consisting of at least one dextran, at least one unfunctionalized dextran, at least one functionalized dextran, at least one unsubstituted dextran, at least one substituted dextran, and combinations thereof.

6. The nanoparticle of claim 1, wherein the at least one polymer is selected from the group consisting of carboxymethyl dextran, at least one dextran, and combinations thereof.

7. The nanoparticle of claim 5, wherein the at least one dextran is selected from the group consisting of a class 1 dextran, a class 2 dextran, a class 3 dextran, and combinations thereof.

8. The nanoparticle of claim 1, wherein the at least one targeting moiety is selected from heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof.

9. The nanoparticle of claim 1, further comprising at least one drug, at least one fluorescent dye, or both.

10. The nanoparticle of claim 9, wherein the at least one drug is selected from the group consisting of docetaxel (DXT), paclitaxel (PXT), bortezomib (Bort), cabozantinib (cabo), brefeldin A (BFA), and combinations thereof.

11. (canceled)

12. The nanoparticle of claim 9, wherein the at least one fluorescent dye is a near infrared fluorescent dye.

13. The nanoparticle of claim 9, wherein the at least one fluorescent dye is selected from the group consisting of DiI, DiR, heptamethine cyanine (HMC), IR820, and combinations thereof.

14. (canceled)

15. (canceled)

16. (canceled)

17. A method for detecting and treating a cancer in a subject, comprising:
administering an effective amount of at least one nanoparticle of claim 9, or an effective amount of at least one nanoparticle of claim 9 to the subject, thereby contacting a tissue of the subject with the at least one nanoparticle such that the at least one nanoparticle binds to the tissue;
detecting the at least one nanoparticle bound to the tissue, wherein the presence of the at least one nanoparticle bound to the tissue is indicative of the cancer in the subject; and
delivering the at least one drug to the tissue thereby treating the cancer in the subject.

18. A method for detecting a cancer in a subject, comprising:
administering an effective amount of at least one nanoparticle of claim 1, or an effective amount of at least one nanoparticle of claim 1 wherein the nanoparticle further comprises at least one fluorescent dye to the subject, thereby contacting a tissue of the subject with the at least one nanoparticle such that the at least one nanoparticle binds to the tissue; and
detecting the at least one nanoparticle bound to the tissue, wherein the presence of the at least one nanoparticle bound to the tissue is indicative of the cancer in the subject.

19. The method of claim 18, further comprising administering a treatment to the subject.

20. The method of claim 17, wherein the nanoparticle is detected by an imaging method selected from the group consisting of magnetic resonance imaging, fluorescence imaging, and combinations thereof.

21. (canceled)

22. The method of claim 17, wherein the cancer is selected from the group consisting of lung cancer, breast cancer, ovarian cancer, pancreatic cancer, head cancer, neck cancer, skin cancer, prostate cancer, brain cancer, and combinations thereof.

23. (canceled)

24. The method of claim 17, wherein the tissue is selected from the group consisting of cancerous tissue, cancer tissue, tumor, tumor tissue, and combinations thereof.

25. The method of claim 17, further comprising administering at least one additional therapy to the subject selected from the group consisting of pharmacological therapy, biological therapy, cell therapy, gene therapy, chemotherapy, radiation therapy, hormonal therapy, surgery, immunotherapy, and combinations thereof.

26. (canceled)

27. The method of claim 19, wherein the treatment is a cancer treatment.

28. A probe comprising at least one coated iron oxide nanoparticle; and at least one targeting moiety, wherein the probe does not comprise boron.

29. The probe of claim 28, wherein the at least one coated iron oxide nanoparticle is selected from the group consisting of Ferumoxytol, Ferumoxides, Ferucarbotran, Ferumoxtran-10, NC100150, VSOP C184, and combinations thereof.

30. The probe of claim 28, wherein the at least one targeting moiety is selected from heptamethine carbocyanine (HMC), modified heptamethine carbocyanine (HMC), unsubstituted heptamethine carbocyanine (HMC), substituted heptamethine carbocyanine (HMC), unfunctionalized heptamethine carbocyanine (HMC), functionalized heptamethine carbocyanine (HMC), glutamate, modified glutamate, unsubstituted glutamate, substituted glutamate, unfunctionalized glutamate, functionalized glutamate, folate, modified folate, unsubstituted folate, substituted folate, unfunctionalized folate, functionalized folate, angiopep, modified angiopep, unsubstituted angiopep, substituted angiopep, unfunctionalized angiopep, functionalized angiopep, and combinations thereof.

31. The probe of claim 28, further comprising at least one drug, at least one fluorescent dye, or both.

32. The probe of claim 31, wherein the at least one drug is selected from the group consisting of docetaxel (DXT), paclitaxel (PXT), bortezomib (Bort), cabozantinib (cabo), brefeldin A (BFA), and combinations thereof.

33. (canceled)

34. The nanoparticle of claim 1, wherein the at least one targeting moiety is selected from an antibody that selectively targets cancer cells, a peptide that selectively targets cancer cells, and combinations thereof.


SEQUENCE LISTING (5 sequences)

SEQ ID NO: 1 - protein, 20 aa, Artificial Sequence
  Synthetic
TFFYGGSRGKRNNFKTEEYC

SEQ ID NO: 2 - protein, 19 aa, Artificial Sequence
  Synthetic
TFFYGGSRGKRNNFKTEEY

SEQ ID NO: 3 - protein, 19 aa, Artificial Sequence
  Synthetic
TFFYGGCRGKRNNFKTEEY

SEQ ID NO: 4 - protein, 19 aa, Artificial Sequence
  Synthetic
TFFYGGSRGKRNNFRTEEY

SEQ ID NO: 5 - protein, 19 aa, Artificial Sequence
  Synthetic
TFFYGGSRGRRNNFRTEEY